SEQUENCE LISTING (15 sequences)

SEQ ID NO: 1 - protein, 5 aa, artificial sequence
  MEDI5884 Heavy Chain CDR1
NYALN

SEQ ID NO: 2 - protein, 17 aa, artificial sequence
  MEDI5884 Heavy Chain CDR2
WINTYSGVGTYAGEFKG

SEQ ID NO: 3 - protein, 11 aa, artificial sequence
  MEDI5884 Heavy Chain CDR3
RGFYGRRFFDV

SEQ ID NO: 4 - protein, 15 aa, artificial sequence
  MEDI5884 Light Chain CDR1
KASQSVDYDVDSYMH

SEQ ID NO: 5 - protein, 7 aa, artificial sequence
  MEDI5884 Light Chain CDR2
AASNLAS

SEQ ID NO: 6 - protein, 9 aa, artificial sequence
  MEDI5884 Light Chain CDR3
QQTIEDPPT

SEQ ID NO: 7 - protein, 120 aa, artificial sequence
  MEDI5884 Variable Heavy  Chain
QVQLVQSGSELKKPGASVKVSCKASGYTFTNYALNWVRQAPGQGLEWMGWINTYSGVGTYAGEFKGRFVFSLDTSVSTAYLQISSLKAEDTAVYYCARRGFYGRRFFDVWGKGTTVTVSS

SEQ ID NO: 8 - protein, 111 aa, artificial sequence
  MEDI5884 Variable Light Chain
DIQLTQSPSSLSASVGDRVTITCKASQSVDYDVDSYMHWYQQKPGKAPKLLIYAASNLASGVPSRFSGSGSGTDFTFTISSLQPEDIATYYCQQTIEDPPTFGGGTKVEIK

SEQ ID NO: 9 - protein, 447 aa, artificial sequence
  MEDI5884 Heavy Chain
QVQLVQSGSELKKPGASVKVSCKASGYTFTNYALNWVRQAPGQGLEWMGWINTYSGVGTYAGEFKGRFVFSLDTSVSTAYLQISSLKAEDTAVYYCARRGFYGRRFFDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 218 aa, artificial sequence
  MEDI5884 Light Chain
DIQLTQSPSSLSASVGDRVTITCKASQSVDYDVDSYMHWYQQKPGKAPKLLIYAASNLASGVPSRFSGSGSGTDFTFTISSLQPEDIATYYCQQTIEDPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 480 aa, Homo Sapiens
SPVPFGPEGRLEDKLHKPKATQTEVKPSVRFNLRTSKDPEHEGCYLSVGHSQPLEDCSFNMTAKTFFIIHGWTMSGIFENWLHKLVSALHTREKDANVVVVDWLPLAHQLYTDAVNNTRVVGHSIARMLDWLQEKDDFSLGNVHLIGYSLGAHVAGYAGNFVKGTVGRITGLDPAGPMFEGADIHKRLSPDDADFVDVLHTYTRSFGLSIGIQMPVGHIDIYPNGGDFQPGCGLNDVLGSIAYGTITEVVKCEHERAVHLFVDSLVNQDKPSFAFQCTDSNRFKKGICLSCRKNRCNSIGYNAKKMRNKRNSKMYLKTRAGMPFRVYHYQMKIHVFSYKNMGEIEPTFYVTLYGTNADSQTLPLEIVERIEQNATNTFLVYTEEDLGDLLKIQLTWEGASQSWYNLWKEFRSYLSQPRNPGRELNIRRIRVKSGETQRKLTFCTEDPENTSISPGRELWFRKCRDGWRMKNETSPTVELP

SEQ ID NO: 12 - protein, 327 aa, artificial sequence
  Constant Region of a Human IgG4P heavy chain
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 107 aa, artificial sequence [sequence omitted]

SEQ ID NO: 14 - protein, 118 aa, artificial sequence
  HS9 variable heavy chain sequence
QVQLVQSGAEVKKPGASVKVSCKASGYTFTSYYMHWVRQAPGQGLEWMGEISPSGGSTSYNQKFQGRVTMTRDTSTSTVYMELSSLRSEDTAVYYCARERPLYASDLWGQGTTVTVSS

SEQ ID NO: 15 - protein, 107 aa, artificial sequence
  HS9 variable light chain sequence
DIQMTQSPSSLSASVGDRVTITCQASQDVKTAVAWYQQKPGKAPKLLIYSASYRYTGVPSRFSGSGSGTDFTFTISSLQPEDIATYYCQQRYSLWRTFGQGTKLEIK

(19) United States
(12) Patent Application Publication
(10) Pub. No.: US 2023/0045712 A1
(43) Pub. Date: Feb. 9, 2023

(54) ENDOTHELIAL LIPASE ANTIBODIES FOR THE TREATMENT OF CARDIOVASCULAR DISEASES

(71) Applicant: MEDIMMUNE, LLC, Gaithersburg, MD (US)

(72) Inventors: Joseph Samuel GRIMSBY, Gaithersburg, MD (US); Chao-Yu JIN, Gaithersburg, MD (US); Judith FALLOON, Gaithersburg, MD (US); Judith Ann HSIA, Gaithersburg, MD (US); Sotirios K. KARATHANASIS, Gaithersburg, MD (US); Richard Thomas GEORGE, Jr., Gaithersburg, MD (US); John Edward LE LAY, Gaithersburg, MD (US); Brian Timothy HUMMER, Gaithersburg, MD (US); Yue HUANG, Gaithersburg, MD (US); Anton I. ROSENBAUM, Gaithersburg, MD (US)

(21) Appl. No.: 17/775,143

(22) PCT Filed: Nov. 6, 2020
(86) PCT No.: PCT/US2020/059458

§ 371 Date: May 6, 2022

Related U.S. Application Data
(60) Provisional application No. 62/932,257, filed on Nov. 7, 2019.
(60) Provisional application No. 62/940,164, filed on Nov. 25, 2019.
(60) Provisional application No. 63/104,410, filed on Oct. 22, 2020.

(51) Int. Cl. C07K 16/40; A61K 9/00; A61P 9/10

(52) U.S. Cl. CPC C07K 16/40; A61K 9/0019; A61P 9/10; A61K 2039/507

(57) ABSTRACT

The present disclosure provides methods of administering antibodies and antigen-binding fragments thereof that specifically bind to human endothelial lipase (EL) to a subject in need thereof, for example, a subject with cardiovascular disease.

### CROSS REFERENCE TO RELATED APPLICATIONS

The application claims the benefit of U.S. Provisional Application Nos. 63/104,410, filed Oct. 22, 2020, 62/940,164, filed Nov. 25, 2019, and 62/932,257, filed Nov. 7, 2019, each of which is hereby incorporated by reference in its entirety.

### 1. FIELD

The present disclosure relates generally to methods of using antibodies and antigen-binding fragments thereof that specifically bind to human endothelial lipase (EL) for the treatment of diseases or disorders, e.g., cardiovascular diseases and disorders. Advantageous dose regimens are provided.

### 2. BACKGROUND

Endothelial lipase (EL) is a circulating phospholipase that has been identified as a member of the triglyceride lipase family. EL has both phospholipase and triglyceride lipase activities, and it hydrolyzes high density lipoproteins (HDL) more efficiently than other lipoproteins. It is believed to play a key role in regulating plasma HDL cholesterol (HDL-C) levels. By hydrolyzing HDL-phospholipids, EL causes HDL particle destabilization and rapid clearance by the kidneys.

Increased plasma EL concentrations have been associated with a deteriorated lipoprotein-lipid profile along with elevated plasma triglyceride and apolipoprotein B concentrations, as well as with smaller low density lipoprotein particle size. (Paradis et al., Can J Cardiol 22: 31B-34B (2006)). Elevated proinflammatory cytokine concentrations and an increased prevalence of the metabolic syndrome have also been observed among individuals with elevated plasma EL concentrations. (Id.) Given these and other factors, EL has been considered to play an important role in cardiovascular disease. (Id.)

Despite the effectiveness of current therapies for cardiovascular disease such as high potency statins, there remains a significant residual risk of major adverse cardiovascular (CV) events in patients with acute coronary syndrome (ACS). The majority of myocardial infarctions (MI) occur in patients with normal low density lipoproteins (LDL) levels, and despite treatment with high dose and highly potent statins, PCSK9 inhibitors, and/or ezetimibe following an MI, the residual risk of a second CV event remains high. For example, the IMPROVE-IT study (ezetimibe+statin) had a 33% risk of a CV event with 7 years of follow-up. In addition, ODYSSEY (PCSK9 inhibitor+statin) has a 9.5% residual risk over 2.8 years.

Low HDL-C levels have been identified as a predictor of atherosclerotic CV events and a coronary heart disease (CHD) risk factor. It has also been hypothesized that HDL particle size and particle number may be useful clinical markers of HDL and associated-disease.

Several attempts to pharmacologically raise HDL levels have been attempted using different mechanisms of action. In particular, four trials of cholesterol transfer protein (CETP) inhibitors have been completed. While CETP inhibitors raise HDL cholesterol, three of the four trials did not improve CV outcomes, and in the one trial that did reduce CV events, the effect was modest with only a 9% relative risk reduction. This approach has been criticized since the inhibition of CETP causes a block in LDL receptor-mediated reverse cholesterol transport.

Humans with partial and complete loss of function mutations in the gene encoding EL exhibit elevated HDL-C, increased cholesterol efflux capacity (CEC), and trends towards reduced CV risk. Thus, neutralization of EL represents a promising therapeutic mechanism. However, currently there no approved therapies that target EL or that sufficiently reduce CV risk. Accordingly, methods of using anti-EL antibodies and antibody-fragments thereof to effectively treat diseases and disorders, e.g., CV diseases and disorders, are needed.

### 3. SUMMARY

Provided herein are methods of treating cardiovascular disease in a subject. In certain aspects, the method comprises administering to the subject about 100 mg to about 350 mg of an antibody or antigen-binding fragment thereof that specifically binds to human endothelial lipase (EL).

Provided herein are methods of reducing atherosclerosis in a subject. In certain aspects, the method comprises administering to the subject about 100 mg to about 350 mg of an antibody or antigen-binding fragment thereof that specifically binds to human EL.

Provided herein are methods of treating cardiovascular disease or reducing atherosclerosis in a subject. In certain aspects, the method comprises administering to the subject an antibody or antigen-binding fragment thereof that specifically binds to EL, wherein the administration of the antibody or antigen-binding fragment thereof: (a) increases high-density lipoprotein cholesterol (HDL-C) in the subject; (b) increases high-density lipoprotein (HDL) particle number in the subject; (c) increases HDL particle size in the subject; (d) increases HDL phospholipids in the subject; (e) increases ApoA1 in the subject; and/or (f) increases cholesterol efflux capacity (CEC) in the subject. In certain aspects, the administration reduces the risk of cardiovascular death, non-fatal myocardial infarction (MI), non-fatal stroke, and/or coronary revascularization in a subject with prior acute coronary syndrome (ACS). In certain aspects, the administration preventing a secondary cardiovascular event in the subject. In certain aspects, the administration reduces the risk of a major adverse cardiovascular event (MACE) in a subject.

Provided herein are methods of reducing the risk of cardiovascular death, non-fatal myocardial infarction (MI), non-fatal stroke, and/or coronary revascularization in a subject with prior acute coronary syndrome (ACS). In certain aspects, the method comprises administering to the subject an antibody or antigen-binding fragment thereof that specifically binds to human EL.

Provided herein are methods of preventing a secondary cardiovascular event in a subject. In certain aspects, the method comprises administering to the subject an antibody or antigen-binding fragment thereof that specifically binds to human EL.

Provided herein are methods of reducing the risk of a major adverse cardiovascular event (MACE) in a subject. In certain aspects, the method comprises administering to the subject an antibody or antigen-binding fragment thereof that specifically binds to human EL.

In certain aspects of the present disclosure, the administration of the antibody or antigen-binding fragment thereof: (a) increases high-density lipoprotein cholesterol (HDL-C) in the subject; (b) increases high-density lipoprotein (HDL) particle number in the subject; (c) increases HDL particle size in the subject; (d) increases HDL phospholipids in the subject; (e) increases ApoA1 in the subject; and/or (f) increases cholesterol efflux capacity (CEC) in the subject.

Provided herein are methods of increasing HDL-C, HDL particle number, HDL particle size, HDL phospholipids, ApoA1, and/or CEC in a subject. In certain aspects, the method comprises administering to the subject an antibody or antigen-binding fragment thereof that specifically binds to EL.

Certain aspects of the present disclosure comprise administering about 100 mg to about 350 mg of the antibody or antigen-binding fragment thereof. Certain aspects of the present disclosure comprise administering about 100 mg, about 110 mg, about 120 mg, about 125 mg, about 130 mg, about 140 mg, about 150 mg, about 160 mg, about 170 mg, about 175 mg, about 180 mg, about 190 mg, about 200 mg, about 210 mg, about 220 mg, about 225 mg, about 230 mg, about 240 mg, about 250 mg, about 260 mg, about 270 mg, about 275 mg, about 280 mg, about 290 mg, about 300 mg, about 310 mg, about 320 mg, about 325 mg, about 330 mg, about 340 mg, or about 350 mg of the antibody or antigen-binding fragment thereof. Certain aspects of the present disclosure comprise administering about 125 mg of the antibody or antigen-binding fragment thereof. Certain aspects of the present disclosure comprise administering 125 mg of the antibody or antigen-binding fragment thereof. Certain aspects of the present disclosure comprise administering about 250 mg of the antibody or antigen-binding fragment thereof. Certain aspects of the present disclosure comprise administering 250 mg of the antibody or antigen-binding fragment thereof. Certain aspects of the present disclosure comprise administering about 200 mg of the antibody or antigen-binding fragment thereof. Certain aspects of the present disclosure comprise administering 200-250 mg of the antibody or antigen-binding fragment thereof.

In certain aspects of the present disclosure, the antibody or antigen-binding fragment thereof is administered once a month. In certain aspects of the present disclosure, the antibody or antigen-binding fragment thereof is administered once a month for at least 3 months. In certain aspects of the present disclosure, the antibody or antigen-binding fragment thereof is administered once a month for at least 12 months or at least 24 months.

In certain aspects of the present disclosure, the antibody or antigen-binding fragment thereof is administered parenterally. In certain aspects of the present disclosure, the antibody or antigen-binding fragment thereof is administered subcutaneously.

In certain aspects of the present disclosure, the antibody or antigen-binding fragment thereof is administered via an accessorized pre-filled syringe (APFS) or an auto-injector.

In certain aspects of the present disclosure, the administration of the antibody or antigen-binding fragment thereof inhibits EL in the subject for 30 days.

In certain aspects of the present disclosure, the administration of the antibody or antigen-binding fragment thereof increases HDL-C in the subject by at least 30%. In certain aspects of the present disclosure, the administration of the antibody or antigen-binding fragment thereof increases HDL-C in the subject by at least 35%. In certain aspects of the present disclosure, the administration of the antibody or antigen-binding fragment thereof increases HDL-C in the subject by at least 40%. In certain aspects of the present disclosure, the administration of the antibody or antigen-binding fragment thereof increases HDL-C in the subject within 30 days of the first administration. In certain aspects of the present disclosure, the administration of the antibody or antigen-binding fragment thereof increases HDL-C in the subject within 90 days of the first administration.

In certain aspects of the present disclosure, the administration of the antibody or antigen-binding fragment thereof increases ApoA1 in the subject by at least 30%. In certain aspects of the present disclosure, the administration of the antibody or antigen-binding fragment thereof increases ApoA1 in the subject by at least 35%. In certain aspects of the present disclosure, the administration of the antibody or antigen-binding fragment thereof increases ApoA1 in the subject within 30 days of the first administration. In certain aspects of the present disclosure, the administration of the antibody or antigen-binding fragment thereof increases ApoA1 in the subject within 90 days of the first administration.

In certain aspects of the present disclosure, the administration of the antibody or antigen-binding fragment thereof increases non-ABCA1 cholesterol efflux capacity in the subject by at least 30%. In certain aspects of the present disclosure, the administration of the antibody or antigen-binding fragment thereof increases non-ABCA1 cholesterol efflux capacity in the subject by at least 35%. In certain aspects of the present disclosure, the administration of the antibody or antigen-binding fragment thereof increases non-ABCA1 cholesterol efflux capacity in the subject within 30 days of the first administration. In certain aspects of the present disclosure, the administration of the antibody or antigen-binding fragment thereof increases non-ABCA1 cholesterol efflux capacity in the subject within 90 days of the first administration.

In certain aspects of the present disclosure, the administration of the antibody or antigen-binding fragment thereof increases HDL particle number in the subject by at least 5% (e.g., as measured using NMR). In certain aspects of the present disclosure, the administration of the antibody or antigen-binding fragment thereof increases HDL particle number in the subject by at least 8% (e.g., as measured using NMR). In certain aspects of the present disclosure, the administration of the antibody or antigen-binding fragment thereof increases HDL particle number in the subject within 30 days of the first administration. In certain aspects of the present disclosure, the administration of the antibody or antigen-binding fragment thereof increases HDL particle number in the subject within 90 days of the first administration.

In certain aspects of the present disclosure, the administration of the antibody or antigen-binding fragment thereof increases HDL particle size in the subject by at least 3%. In certain aspects of the present disclosure, the administration of the antibody or antigen-binding fragment thereof increases HDL particle size in the subject by at least 5%. In certain aspects of the present disclosure, the administration of the antibody or antigen-binding fragment thereof increases HDL particle size in the subject within 30 days of the first administration. In certain aspects of the present disclosure, the administration of the antibody or antigen-binding fragment thereof increases HDL particle size in the subject within 90 days of the first administration.

In certain aspects of the present disclosure, the administration of the antibody or antigen-binding fragment thereof increases HDL phospholipids in the subject by at least 50%. In certain aspects of the present disclosure, the administration of the antibody or antigen-binding fragment thereof increases HDL phospholipids in the subject within 30 days of the first administration. In certain aspects of the present disclosure, the administration of the antibody or antigen-binding fragment thereof increases HDL phospholipids in the subject within 90 days of the first administration.

In certain aspects of the present disclosure, the administration of the antibody or antigen-binding fragment thereof increases plasma phosphatidylinositol (PI) levels in the subject by at least 100% or by at least 250%. In certain aspects of the present disclosure, the increased plasma PI levels are increased PI(14:2/20:0), PI(14:2/22:0), PI(14:2/22:1), PI(14:2/22:2), PI(16:0/16:1), PI(16:0/18:0), PI(16:0/18:2), PI(16:0/20:2), PI(16:0/20:3), PI(16:0/20:4), PI(16:0/22:4), PI(16:1/18:0), PI(16:1/18:1), PI(18:0/18:0), PI(18:0/18:1), PI(18:0/18:2), PI(18:0/18:3), PI(18:0/20:2), PI(18:0/20:3), PI(18:0/20:4), PI(18:0/22:4), PI(18:0/22:5), PI(18:0/22:6), PI(18:1/16:0), PI(18:1/18:1), PI(18:1/18:2), PI(18:1/20:2), PI(18:1/20:3), PI(18:1/20:4), and/or PI(18:2/18:2) levels. In certain aspects of the present disclosure, the administration of the antibody or antigen-binding fragment thereof increases plasma phosphatidylinositol (PI) levels in the subject within 90 days of the first administration.

In certain aspects of the present disclosure, the subject has a cardiovascular disease. In certain aspects of the present disclosure, the cardiovascular disease is coronary artery disease, coronary heart disease (CHD), chronic arterial disease, cerebrovascular disease, atherosclerotic cardiovascular disease, or peripheral artery disease.

In certain aspects of the present disclosure, the subject has stable coronary artery disease or stable coronary heart disease. In certain aspects of the present disclosure, the subject has prior acute coronary syndrome (ACS).

In certain aspects of the present disclosure, the subject is receiving statin therapy. In certain aspects of the present disclosure, the subject is not receiving statin therapy.

In certain aspects of the present disclosure, the subject has triglyceride levels ≤500 mg/dL prior to the administration. In certain aspects of the present disclosure, the subject has LDL-C≤100 mg/dL prior to the administration.

In certain aspects of the present disclosure, the subject is human.

In certain aspects of the present disclosure, the antibody or antigen-binding fragment thereof neutralizes EL activity.

In certain aspects of the present disclosure, the antibody or antigen-binding fragment thereof has reduced effector function. In certain aspects of the present disclosure, the antibody or antigen-binding fragment thereof does not have antibody-dependent cell-mediated cytotoxicity (ADCC) activity. In certain aspects of the present disclosure, the antibody does not have complement-dependent cytotoxicity (CDC) activity.

In certain aspects of the present disclosure, the antibody binds to cynomolgus monkey EL.

In certain aspects of the present disclosure, the antibody or antigen-binding fragment thereof competitively inhibits binding to EL of an antibody comprising a VH comprising the amino acid sequence set forth in SEQ ID NO:7 and a VL comprising the amino acid sequence set forth in SEQ ID NO:8. In certain aspects of the present disclosure, the antibody or antigen-binding fragment thereof binds to the same epitope of EL as an antibody comprising a VH comprising the amino acid sequence set forth in SEQ ID NO:7 and a VL comprising the amino acid sequence set forth in SEQ ID NO:8.

In certain aspects of the present disclosure, the antibody or antigen-binding fragment thereof comprises the heavy chain variable region (VH) complementarity determining region (CDR) 1, VH CDR2, VH CDR3, VL CDR1, VL CDR2, and the light chain variable region (VL) CDR3 of sequences of MEDI5884. In certain aspects of the present disclosure, the CDRs are the Kabat-defined CDRs, the Chothia-defined CDRs, or the AbM-defined CDRs. In certain aspects of the present disclosure, the antibody or antigen-binding fragment thereof comprises a VH CDR1 comprising the amino acid sequence of SEQ ID NO:1, a VH CDR2 comprising the amino acid sequence of SEQ ID NO:2, a VH CDR3 comprising the amino acid sequence of SEQ ID NO:3, VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:6.

In certain aspects of the present disclosure, the antibody or antigen-binding fragment thereof comprises a VH comprising the amino acid sequence set forth in SEQ ID NO:7 and/or a VL comprising the amino acid sequence set forth in SEQ ID NO:8.

In certain aspects of the present disclosure, the antibody or antigen-binding fragment comprises an IgG heavy chain constant region. In certain aspects of the present disclosure, the IgG heavy chain constant region is an IgG4 heavy chain constant region. In certain aspects of the present disclosure, the IgG4 heavy chain constant region is a IgG4P heavy chain constant region. In certain aspects of the present disclosure, the antibody or antigen-binding fragments thereof comprises a kappa light chain constant region.

In certain aspects of the present disclosure, the antibody or antigen-binding fragment comprises a heavy chain constant region and/or a light chain constant region. In certain aspects of the present disclosure, the heavy chain constant region is a human IgG4P heavy chain constant region and/or wherein the light chain constant region is a human IgGκ light chain constant region.

In certain aspects of the present disclosure, the antibody or antigen-binding fragment thereof is a humanized antibody or antigen-binding fragment thereof.

In certain aspects of the present disclosure, the antibody or antigen-binding fragment thereof comprises a heavy chain constant region comprising the amino acid sequence set forth in SEQ ID NO:12 and/or a light chain constant region comprising the amino acid sequence set forth in SEQ ID NO:13.

In certain aspects of the present disclosure, the antibody comprises a heavy chain comprising the amino acid sequence set forth in SEQ ID NO:9 and a light chain comprising the amino acid sequence set forth in SEQ ID NO:10.

In certain aspects of the present disclosure, the antibody or antigen-binding fragment thereof is a full length antibody.

In certain aspects of the present disclosure, the antibody or antigen-binding fragment thereof is an antigen binding fragment. In certain aspects of the present disclosure, the antigen binding fragment comprises a Fab, Fab′, F(ab′)₂, single chain Fv (scFv), disulfide linked Fv, V-NAR domain, IgNar, intrabody, IgGΔCH2, minibody, F(ab′)₃, tetrabody, triabody, diabody, single-domain antibody, DVD-Ig, Fcab, mAb², (scFv)₂, or scFv-Fc.

Provided herein are methods of treating cardiovascular disease in a subject. In certain aspects, the method comprises subcutaneously administering 250 mg of an antibody or antigen-binding fragment thereof to the subject once a month, wherein the antibody or antigen-binding fragment specifically binds to human EL and comprises a VH comprising the amino acid sequence set forth in SEQ ID NO:7 and a VL comprising the amino acid sequence set forth in SEQ ID NO:8. In certain aspects, the method comprises subcutaneously administering about 200 mg of an antibody or antigen-binding fragment thereof to the subject once a month, wherein the antibody or antigen-binding fragment specifically binds to human EL and comprises a VH comprising the amino acid sequence set forth in SEQ ID NO:7 and a VL comprising the amino acid sequence set forth in SEQ ID NO:8. In certain aspects, the method comprises subcutaneously administering 200-250 mg of an antibody or antigen-binding fragment thereof to the subject once a month, wherein the antibody or antigen-binding fragment specifically binds to human EL and comprises a VH comprising the amino acid sequence set forth in SEQ ID NO:7 and a VL comprising the amino acid sequence set forth in SEQ ID NO:8. Provided herein are methods of reducing the risk of cardiovascular death, non-fatal myocardial infarction (MI), non-fatal stroke, and/or coronary revascularization in a subject with prior acute coronary syndrome (ACS). In certain aspects, the method comprises subcutaneously administering 250 mg of an antibody or antigen-binding fragment thereof to the subject once a month, wherein the antibody or antigen-binding fragment specifically binds to human EL and comprises a VH comprising the amino acid sequence set forth in SEQ ID NO:7 and a VL comprising the amino acid sequence set forth in SEQ ID NO:8. In certain aspects, the method comprises subcutaneously administering about 200 mg of an antibody or antigen-binding fragment thereof to the subject once a month, wherein the antibody or antigen-binding fragment specifically binds to human EL and comprises a VH comprising the amino acid sequence set forth in SEQ ID NO:7 and a VL comprising the amino acid sequence set forth in SEQ ID NO:8. In certain aspects, the method comprises subcutaneously administering 200-250 mg of an antibody or antigen-binding fragment thereof to the subject once a month, wherein the antibody or antigen-binding fragment specifically binds to human EL and comprises a VH comprising the amino acid sequence set forth in SEQ ID NO:7 and a VL comprising the amino acid sequence set forth in SEQ ID NO:8.

In certain aspects of the present disclosure, the antibody comprises the heavy chain constant region amino acid sequence set forth in SEQ ID NO:9 and the light chain constant region amino acid sequence set forth in SEQ ID NO:10.

In certain aspects of the present disclosure, the method further comprises administering an inhibitor of proprotein convertase subtilisin/kexin type 9 (PCSK9). In certain aspects, the administration of the antibody or antigen-binding fragment thereof that specifically binds to human EL and the administration of the inhibitor of PCSK9 are simultaneous. In certain aspects, the antibody or antigen-binding fragment thereof that specifically binds to human EL and the inhibitor of PCSK9 are administered in separate pharmaceutical compositions. In certain aspects, the administration of the antibody or antigen-binding fragment thereof that specifically binds to human EL and the administration of the inhibitor of PCSK9 are sequential.

In certain aspects, the inhibitor of PCSK9 is an anti-PCSK9 antibody or antigen-binding fragment thereof. In certain aspects, the inhibitor of PCSK9 is HS9, evolocumab, alirocumab, or bococizumab.

### 4. BRIEF DESCRIPTION OF THE FIGURES

FIG. 1A shows dose-dependent increases in exposure to MEDI5584. (See Examples 4 and 5.)

FIG. 1B shows MEDI5884-dose-dependent target engagement (inhibition of EL). (See Examples 4 and 5.)

FIG. 2 shows MEDI5884-dose-dependent increase in high-density lipoprotein cholesterol (HDL-C). (See Examples 4 and 5.)

FIG. 3 shows MEDI5884-dose-dependent increase in apolipoprotein A1 (ApoA1). (See Examples 4 and 5.)

FIG. 4 shows MEDI5884-dose-dependent increase in high-density lipoprotein phospholipid (HDL-PL). (See Examples 4 and 5.)

FIG. 5 shows MEDI5884-dose-dependent increase in non-ATP-binding cassette transporter A1 (ABCA1) cholesterol efflux. (See Example 4.)

FIG. 6 shows the dose-response relationships of HDL-C, ApoA1, and HDL-PL based on area-under-effect-curve during Day 60 and Day 90 (AUEC_{d60-90}). (See Example 5.)

FIG. 7 shows the pharmacokinetic (PK) and pharmacodynamic (PD) modeling for MEDI5884. CLd=intercompartmental clearance; CL=apparent systemic clearance; conc=concentration; dHDL(t)/dt=change in HDL over time; HDL=high-density lipoprotein cholesterol; HDL(t)=HDL levels at time t; IC50=concentration to reach 50% of maximal effect (estimated simultaneous with Km); IH=inhibition effect from MEDI5884; Imax=maximal inhibition effect; Ka=rate of absorption; Kin=HDL production rate; Km=concentration to reach 50% of Vmax; Kout=HDL elimination rate; SC=subcutaneous; Vmax=maximum contribution of dose-dependent nonlinear clearance. (See Example 5.)

FIG. 8 shows PK simulations of MEDI5884 dosed at 250 mg monthly. (See Example 5.)

FIG. 9 shows the study design for analyzing the effect of the combined inhibition of EL and proprotein convertase subtilisin/kexin type 9 (PCSK9) in cynomolgus monkeys.

FIG. 10 shows the effect of combined inhibition of EL and PCSK9 on LDL-C and HDL-C levels in cynomolgus monkeys. Data are presented as average change from baseline (Day 0) measurements+/−SEM. The mean baseline value (n=16) is indicated in each plot. (See Example 6.)

FIG. 11 shows the effect of combined inhibition of EL and PCSK9 on ApoB and ApoA1 levels in cynomolgus monkeys. Data are presented as average change from baseline (Day 0) measurements+/−SEM. The mean baseline value (n=16) is indicated in each plot. (See Example 6.)

FIG. 12 shows the effect of combined inhibition of EL and PCSK9 on cholesterol efflux capacity (global efflux and ABCA1 efflux) in cynomolgus monkeys. (See Example 6.)

FIGS. 13A-C show the time course and dose-response of plasma phosphatidylinositol (PI) species for selected doses in trials with single ascending doses (SAD) and multiple ascending doses (MAD) in healthy patients and in coronary artery disease (CAD) patients. (See Example 7.)

FIGS. 14A-C show the time course and dose-response of plasma phosphatidylinositol (PI) species for all doses in trials with single ascending doses (SAD) and multiple ascending doses (MAD) in healthy patients and in coronary artery disease (CAD) patients. (See Example 7.)

FIG. 15 shows the effect of MEDI5884 on plasma phosphatidylinositol (PI) species in healthy volunteers and CAD patients on Day 21. (See Example 7.)

FIG. 16 shows the effect of MEDI5884 on plasma phosphatidylinositol (PI) species in healthy volunteers and coronary artery disease (CAD) patients on all days. (See Example 7.)

FIGS. 17A-E show the percent change from baseline for various plasma phosphatidylinositol (PI) species in coronary artery disease (CAD) patients treated with various doses of MEDI5884 or with placebo. (See Example 7.)

FIG. 18 shows the average percent change from baseline for all measured plasma phosphatidylinositol (PI) species in coronary artery disease (CAD) patients treated with various doses of MEDI5884 or with placebo. (See Example 7.)

FIGS. 19A-E show the percent change from baseline for various plasma phosphatidylinositol (PI) species in healthy volunteers treated with various doses of MEDI5884 or with placebo. (See Example 7.)

### 5. DETAILED DESCRIPTION

Provided herein are methods of administering antibodies (e.g., monoclonal antibodies) and antigen-binding fragments thereof that specifically bind to endothelial lipase (EL, e.g., human EL). The anti-EL antibodies and antigen-binding fragments thereof can be administered, for example, to treat cardiovascular disease in a subject. The anti-EL antibody or antigen-binding fragments thereof can increase high-density lipoprotein cholesterol (HDL-C), increase HDL particle number, increase HDL particle size, increase HDL phospholipids, increase ApoA1, and/or increase cholesterol efflux capacity in a subject. In some aspects of the present disclosure, about 100 mg to about 350 mg, (e.g., about 250 mg) of the antibody or antigen-binding fragment thereof is administered to the subject, e.g., wherein the administration occurs about once every month (QM).

### 5.1 Terminology

As used herein, the term “endothelial lipase” or “EL” refers to mammalian EL polypeptides including, but not limited to, native EL polypeptides and isoforms of EL polypeptides. “EL” encompasses full-length, unprocessed EL polypeptides as well as forms of EL polypeptides that result from processing within the cell. As used herein, the term “human EL” refers to a polypeptide comprising the amino acid sequence of SEQ ID NO:11. An “EL polynucleotide,” “EL nucleotide,” or “EL nucleic acid” refer to a polynucleotide encoding EL.

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, chimeric antibodies, humanized antibodies, human antibodies, fusion proteins comprising an antibody, and any other modified immunoglobulin molecule so long as the antibodies exhibit the desired biological activity. An antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes, etc.

The term “antibody fragment” refers to a portion of an intact antibody. An “antigen-binding fragment,” “antigen-binding domain,” or “antigen-binding region,” refers to a portion of an intact antibody that binds to an antigen. An antigen-binding fragment can contain an antigen recognition site of an intact antibody (e.g., complementarity determining regions (CDRs) sufficient to specifically bind antigen). Examples of antigen-binding fragments of antibodies include, but are not limited to Fab, Fab′, F(ab′)2, and Fv fragments, linear antibodies, and single chain antibodies. An antigen-binding fragment of an antibody can be derived from any animal species, such as rodents (e.g., mouse, rat, or hamster) and humans or can be artificially produced.

The terms “anti-EL antibody,” “EL antibody” and “antibody that binds to EL” refer to an antibody that is capable of specifically binding EL with sufficient affinity such that the antibody is useful as a diagnostic and/or therapeutic agent in targeting EL. As used herein, the terms “specifically binding,” “immunospecifically binding,” “immunospecifically recognizing,” and “specifically recognizing” are analogous terms in the context of antibodies or antigen-binding fragments thereof. These terms indicate that the antibody or antigen-binding fragment thereof binds to an epitope via its antigen-binding domain and that the binding entails some complementarity between the antigen binding domain and the epitope. Accordingly, an antibody that “specifically binds” to human EL (SEQ ID NO:11) can also bind to EL from other species (e.g., cynomolgus monkey) and/or EL proteins produced from other human alleles, but the extent of binding to an un-related, non-EL protein (e.g., other lipases such as hepatic lipase or lipoprotein lipase) is less than about 10% of the binding of the antibody to EL as measured, e.g., by an in vitro neutralization assay.

A “monoclonal” antibody or antigen-binding fragment thereof refers to a homogeneous antibody or antigen-binding fragment population involved in the highly specific binding of a single antigenic determinant, or epitope. This is in contrast to polyclonal antibodies that typically include different antibodies directed against different antigenic determinants. The term “monoclonal” antibody or antigen-binding fragment thereof encompasses both intact and full-length monoclonal antibodies as well as antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, “monoclonal” antibody or antigen-binding fragment thereof refers to such antibodies and antigen-binding fragments thereof made in any number of manners including but not limited to by hybridoma, phage selection, recombinant expression, and transgenic animals.

As used herein, the terms “variable region” or “variable domain” are used interchangeably and are common in the art. The variable region typically refers to a portion of an antibody, generally, a portion of a light or heavy chain, typically about the amino-terminal 110 to 120 amino acids or 110 to 125 amino acids in the mature heavy chain and about 90 to 115 amino acids in the mature light chain, which differ in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that CDRs of the light and heavy chains are primarily responsible for the interaction and specificity of the antibody with antigen. In some aspects of the present disclosure, the variable region is a human variable region. In some aspects of the present disclosure, the variable region comprises rodent or murine CDRs and human framework regions (FRs). In particular aspects of the present disclosure, the variable region is a primate (e.g., non-human primate) variable region. In some aspects of the present disclosure, the variable region comprises rodent or murine CDRs and primate (e.g., non-human primate) framework regions (FRs).

The terms “VL” and “VL domain” are used interchangeably to refer to the light chain variable region of an antibody.

The terms “VH” and “VH domain” are used interchangeably to refer to the heavy chain variable region of an antibody.

The term “Kabat numbering” and like terms are recognized in the art and refer to a system of numbering amino acid residues in the heavy and light chain variable regions of an antibody or an antigen-binding fragment thereof. In some aspects, CDRs can be determined according to the Kabat numbering system (see, e.g., Kabat E A & Wu T T (1971) Ann NY Acad Sci 190: 382-391 and Kabat E A et al., (1991) Sequences of Proteins of Immunological Interest, Fifth Edition, U.S. Department of Health and Human Services, NIH Publication No. 91-3242). Using the Kabat numbering system, CDRs within an antibody heavy chain molecule are typically present at amino acid positions 31 to 35, which optionally can include one or two additional amino acids, following 35 (referred to in the Kabat numbering scheme as 35A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3). In some aspects of the present disclosure, the CDRs of the antibodies described herein have been determined according to the Kabat numbering scheme.

Chothia refers instead to the location of the structural loops (Chothia and Lesk, J. Mol. Biol. 196:901-917 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software.

| Loop | Kabat | AbM | Chothia |
|---|---|---|---|
| LI | L24-L34 | L24-L34 | L24-L34 |
| L2 | L50-L56 | L50-L56 | L50-L56 |
| L3 | L89-L97 | L89-L97 | L89-L97 |
| H1 | H31-H35B | H26-H35B | H26-H32 . . . 34 |
| | | (Kabat Numbering) | |
| H1 | H31-H35 | H26-H35 | H26-H32 |
| | | (Chothia Numbering) | |
| H2 | H50-H65 | H50-H58 | H52-H56 |
| H3 | H95-H102 | H95-H102 | H95-H102 |

As used herein, the term “constant region” and “constant domain” are interchangeable and have their common meanings in the art. The constant region is an antibody portion, e.g., a carboxyl terminal portion of a light and/or heavy chain which is not directly involved in binding of an antibody to antigen but which can exhibit various effector functions, such as interaction with the Fc receptor. The constant region of an immunoglobulin molecule generally has a more conserved amino acid sequence relative to an immunoglobulin variable domain.

As used herein, the term “heavy chain” when used in reference to an antibody can refer to any distinct type, e.g., alpha (α), delta (δ), epsilon (ε), gamma (γ), and mu (μ), based on the amino acid sequence of the constant domain, which give rise to IgA, IgD, IgE, IgG, and IgM classes of antibodies, respectively, including subclasses of IgG, e.g., IgG₁, IgG₂, IgG₃, and IgG₄. Heavy chain amino acid sequences are well known in the art. In some aspects of the present disclosure, the heavy chain is a human heavy chain.

As used herein, the term “light chain” when used in reference to an antibody can refer to any distinct type, e.g., kappa (κ) or lambda (λ) based on the amino acid sequence of the constant domains. Light chain amino acid sequences are well known in the art. In some aspects of the present disclosure, the light chain is a human light chain.

The term “MEDI5884” refers to anti-EL antibody that comprises the heavy chain of SEQ ID NO:9 and the light chain of SEQ ID NO:10. MEDI5884 is also referred to as “S6F1-4P,” and it comprises the heavy chain variable region of the h55A1-S6 antibody and the light chain variable region of the h55A1-F1 antibody, which are disclosed in US Published Application No. 2017/0260290, which is herein incorporated by reference in its entirety.

The term “chimeric” antibodies or antigen-binding fragments thereof refers to antibodies or antigen-binding fragments thereof wherein the amino acid sequence is derived from two or more species. Typically, the variable region of both light and heavy chains corresponds to the variable region of antibodies or antigen-binding fragments thereof derived from one species of mammals (e.g. mouse, rat, rabbit, etc.) with the desired specificity, affinity, and capability while the constant regions are homologous to the sequences in antibodies or antigen-binding fragments thereof derived from another (usually human) to avoid eliciting an immune response in that species.

The term “humanized” antibody or antigen-binding fragment thereof refers to forms of non-human (e.g. murine) antibodies or antigen-binding fragments that are specific immunoglobulin chains, chimeric immunoglobulins, or fragments thereof that contain minimal non-human (e.g., murine) sequences. Typically, humanized antibodies or antigen-binding fragments thereof are human immunoglobulins in which residues from the complementary determining region (CDR) are replaced by residues from the CDR of a non-human species (e.g. mouse, rat, rabbit, hamster) that have the desired specificity, affinity, and capability (“CDR grafted”) (Jones et al., Nature 321:522-525 (1986); Riechmann et al., Nature 332:323-327 (1988); Verhoeyen et al., Science 239:1534-1536 (1988)). In some instances, certain Fv framework region (FR) residues of a human immunoglobulin are replaced with the corresponding residues in an antibody or fragment from a non-human species that has the desired specificity, affinity, and capability. The humanized antibody or antigen-binding fragment thereof can be further modified by the substitution of additional residues either in the Fv framework region and/or within the non-human CDR residues to refine and optimize antibody or antigen-binding fragment thereof specificity, affinity, and/or capability. In general, the humanized antibody or antigen-binding fragment thereof will comprise variable domains containing all or substantially all of the CDR regions that correspond to the non-human immunoglobulin whereas all or substantially all of the FR regions are those of a human immunoglobulin consensus sequence. The humanized antibody or antigen-binding fragment thereof can also comprise at least a portion of an immunoglobulin constant region or domain (Fc), typically that of a human immunoglobulin. Examples of methods used to generate humanized antibodies are described in U.S. Pat. No. 5,225,539; Roguska et al., Proc. Natl. Acad. Sci., USA, 91(3):969-973 (1994), and Roguska et al., Protein Eng. 9(10):895-904 (1996). In some aspects of the present disclosure, a “humanized antibody” is a resurfaced antibody.

The term “human” antibody or antigen-binding fragment thereof means an antibody or antigen-binding fragment thereof having an amino acid sequence derived from a human immunoglobulin gene locus, where such antibody or antigen-binding fragment is made using any technique known in the art. This definition of a human antibody or antigen-binding fragment thereof includes intact or full-length antibodies and fragments thereof.

“Binding affinity” generally refers to the strength of the sum total of non-covalent interactions between a single binding site of a molecule (e.g., an antibody or antigen-binding fragment thereof) and its binding partner (e.g., an antigen). Unless indicated otherwise, as used herein, “binding affinity” refers to intrinsic binding affinity which reflects a 1:1 interaction between members of a binding pair (e.g., antibody or antigen-binding fragment thereof and antigen). The affinity of a molecule X for its partner Y can generally be represented by the dissociation constant (K_{D}). Affinity can be measured and/or expressed in a number of ways known in the art, including, but not limited to, equilibrium dissociation constant (K_{D}), and equilibrium association constant (K_{A}). The K_{D} is calculated from the quotient of k_{off}/kₒₙ, whereas K_{A} is calculated from the quotient of kₒₙ/k_{off}. kₒₙ refers to the association rate constant of, e.g., an antibody or antigen-binding fragment thereof to an antigen, and k_{off} refers to the dissociation of, e.g., an antibody or antigen-binding fragment thereof from an antigen. The kₒₙ, and k_{off} can be determined by techniques known to one of ordinary skill in the art, such as BIAcore® or KinExA.

As used herein, an “epitope” is a term in the art and refers to a localized region of an antigen to which an antibody or antigen-binding fragment thereof can specifically bind. An epitope can be, for example, contiguous amino acids of a polypeptide (linear or contiguous epitope) or an epitope can, for example, come together from two or more non-contiguous regions of a polypeptide or polypeptides (conformational, non-linear, discontinuous, or non-contiguous epitope). In some aspects of the present disclosure, the epitope to which an antibody or antigen-binding fragment thereof specifically binds can be determined by, e.g., NMR spectroscopy, X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g., site-directed mutagenesis mapping). For X-ray crystallography, crystallization can be accomplished using any of the known methods in the art (e.g., Giegé R et al., (1994) Acta Crystallogr D Biol Crystallogr 50(Pt 4): 339-350; McPherson A (1990) Eur J Biochem 189: 1-23; Chayen N E (1997) Structure 5: 1269-1274; McPherson A (1976) J Biol Chem 251: 6300-6303). Antibody/antigen-binding fragment thereof: antigen crystals can be studied using well known X-ray diffraction techniques and can be refined using computer software such as X-PLOR (Yale University, 1992, distributed by Molecular Simulations, Inc.; see, e.g., Meth Enzymol (1985) volumes 114 & 115, eds Wyckoff H W et al.; U.S. 2004/0014194), and BUSTER (Bricogne G (1993) Acta Crystallogr D Biol Crystallogr 49(Pt 1): 37-60; Bricogne G (1997) Meth Enzymol 276A: 361-423, ed Carter C W; Roversi P et al., (2000) Acta Crystallogr D Biol Crystallogr 56(Pt 10): 1316-1323). Mutagenesis mapping studies can be accomplished using any method known to one of skill in the art. See, e.g., Champe M et al., (1995) J Biol Chem 270: 1388-1394 and Cunningham B C & Wells J A (1989) Science 244: 1081-1085 for a description of mutagenesis techniques, including alanine scanning mutagenesis techniques.

An antibody that “binds to the same epitope” as a reference antibody refers to an antibody that binds to the same amino acid residues as the reference antibody. The ability of an antibody to bind to the same epitope as a reference antibody can determined by a hydrogen/deuterium exchange assay (see Coales et al. Rapid Commun. Mass Spectrom. 2009; 23: 639-647) or x-ray crystallography.

An antibody is said to “competitively inhibit” binding of a reference antibody to a given epitope if it preferentially binds to that epitope or an overlapping epitope to the extent that it blocks, to some degree, binding of the reference antibody to the epitope. Competitive inhibition can be determined by any method known in the art, for example, competition ELISA assays. An antibody can be said to competitively inhibit binding of the reference antibody to a given epitope by at least 90%, at least 80%, at least 70%, at least 60%, or at least 50%.

As used herein, an “inhibitor of PCSK9” is an agent that blocks the interaction of PCSK9 with LDL receptor.

A polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cell or compositions include those which have been purified to a degree that they are no longer in a form in which they are found in nature. In some aspects of the present disclosure, an antibody, polynucleotide, vector, cell, or composition which is isolated is substantially pure. As used herein, “substantially pure” refers to material which is at least 50% pure (i.e., free from contaminants), at least 90% pure, at least 95% pure, at least 98% pure, or at least 99% pure.

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this disclosure are based upon antibodies, in some aspects of the present disclosure, the polypeptides can occur as single chains or associated chains.

As used herein, the term “host cell” can be any type of cell, e.g., a primary cell, a cell in culture, or a cell from a cell line. In some aspects of the present disclosure, the term “host cell” refers to a cell transfected with a nucleic acid molecule and the progeny or potential progeny of such a cell. Progeny of such a cell can be non-identical to the parent cell transfected with the nucleic acid molecule, e.g., due to mutations or environmental influences that can occur in succeeding generations or integration of the nucleic acid molecule into the host cell genome.

The term “pharmaceutical formulation” refers to a preparation which is in such form as to permit the biological activity of the active ingredient to be effective, and which contains no additional components which are unacceptably toxic to a subject to which the formulation would be administered. The formulation can be sterile.

The terms “administer,” “administering,” “administration,” and the like, as used herein, refer to methods that can be used to enable delivery of a drug, e.g., an anti-EL antibody or antigen-binding fragment thereof to the desired site of biological action (e.g., intravenous administration). Administration techniques that can be employed with the agents and methods described herein are found in e.g., Goodman and Gilman, The Pharmacological Basis of Therapeutics, current edition, Pergamon; and Remington's, Pharmaceutical Sciences, current edition, Mack Publishing Co., Easton, Pa.

As used herein, the terms “subject” and “patient” are used interchangeably. The subject can be an animal. In some aspects of the present disclosure, the subject is a mammal such as a non-human animal (e.g., cow, pig, horse, cat, dog, rat, mouse, monkey or other primate, etc.). In some aspects of the present disclosure, the subject is a cynomolgus monkey. In some aspects of the present disclosure, the subject is a human.

The term “therapeutically effective amount” refers to an amount of a drug, e.g., an anti-EL antibody or antigen-binding fragment thereof, effective to treat a disease or disorder in a subject. Terms such as “treating,” “treatment,” “to treat,” “alleviating,” and “to alleviate” refer to therapeutic measures that cure, slow down, lessen symptoms of, and/or halt progression of a pathologic condition or disorder. Thus, those in need of treatment include those already diagnosed with or suspected of having the disorder. Administration “in combination with” one or more further therapeutic agents includes simultaneous (concurrent) or consecutive administration in any order.

As used in the present disclosure and claims, the singular forms “a,” “an,” and “the” include plural forms unless the context clearly dictates otherwise.

It is understood that wherever aspects of the present disclosure are described herein with the language “comprising,” otherwise analogous aspects described in terms of “consisting of” and/or “consisting essentially of” are also provided.

Unless specifically stated or obvious from context, as used herein, the term “or” is understood to be inclusive. The term “and/or” as used in a phrase such as “A and/or B” herein is intended to include both “A and B,” “A or B,” “A,” and “B.” Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, the terms “about” and “approximately,” when used to modify a numeric value or numeric range, indicate that deviations of 5% to 10% above and 5% to 10% below the value or range remain within the intended meaning of the recited value or range.

Any compositions or methods provided herein can be combined with one or more of any of the other compositions and methods provided herein.

### 5.2 Methods of Treatment Using Anti-EL Antibodies or Antigen-Binding Fragments Thereof

Provided herein are methods of administering an anti-EL antibody or antigen-binding fragment thereof described herein or a pharmaceutical composition thereof as described herein to a subject in need thereof.

As provided herein, administration of an anti-EL antibody or antigen-binding fragment thereof or a pharmaceutical composition thereof can treat cardiovascular disease in a subject (e.g., a human subject). The cardiovascular disease can be, for example, coronary artery disease, coronary heart disease, cerebrovascular disease, or peripheral artery disease.

Administration of an anti-EL antibody or antigen-binding fragment thereof or a pharmaceutical composition thereof can reduce or prevent atherosclerosis in a subject (e.g., a human subject).

Administration of an anti-EL antibody or antigen-binding fragment thereof or a pharmaceutical composition thereof can prevent or reduce the risk of a secondary cardiovascular event in a subject (e.g., a human subject).

Administration of an anti-EL antibody or antigen-binding fragment thereof or a pharmaceutical composition thereof can reduce the risk of cardiovascular death, non-fatal myocardial infarction (MI), non-fatal stroke, coronary revascularization, or any combination thereof in a subject (e.g., a human subject). The subject can, for example, be a subject with a prior acute coronary syndrome (ACS).

Administration of an anti-EL antibody or antigen-binding fragment thereof or a pharmaceutical composition thereof can prevent or reduce the risk of a major cardiovascular event (MACE) in a subject (e.g., a human subject).

Administration of an anti-EL antibody or antigen-binding fragment thereof or a pharmaceutical composition thereof can (i) increase high-density lipoprotein cholesterol (HDL-C); (ii) increase high-density lipoprotein (HDL) particle number; (iii) increase HDL particle size; (iv) increase HDL phospholipids; (v) increase ApoA1; (vi) increase cholesterol efflux capacity (CEC); or (vii) any combination thereof.

Administration of an anti-EL antibody or antigen-binding fragment thereof or a pharmaceutical composition thereof can also increase plasma phosphatidylinositol (PI) levels.

In some aspects, administration of the anti-EL antibody or antigen-binding fragment thereof inhibits EL.

In some aspects, administration of the anti-EL antibody or antigen-binding fragment thereof increases HDL-C in the subject. The administration of the anti-EL antibody or antigen-binding fragment thereof can increase HDL-C by, for example, at least 30%, at least 35%, or at least 40%. Thus, the administration of the anti-EL antibody or antigen-binding fragment thereof can increase HDL-C by about 30% to about 125%, by about 30% to about 100%, by about 30% to about 75%, by about 30% to about 50%, by about 30% to about 45% or by about 30% to about 40%. The increase in HDL-C can occur within 30 days of the first administration, within 60 days of the first administration, or within 90 days of the first administration.

In some aspects, administration of the anti-EL antibody or antigen-binding fragment thereof increases HDL particle number in the subject. The administration of the anti-EL antibody or antigen-binding fragment thereof can increase HDL particle number by, for example, at least 5%, at least 8%, at least 10%, or at least 15% (e.g., as measured using NMR). Thus, the administration of the anti-EL antibody or antigen-binding fragment thereof can increase HDL particle number by about 5% to about 20% or by about 5% to about 18% (e.g., as measured using NMR). The increase in HDL particle number can occur within 30 days of the first administration, within 60 days of the first administration, or within 90 days of the first administration.

In some aspects, administration of the anti-EL antibody or antigen-binding fragment thereof increases HDL particle size in the subject. The administration of the anti-EL antibody or antigen-binding fragment thereof can increase HDL particle size by, for example, at least 3% or at least 5%. Thus, the administration of the anti-EL antibody or antigen-binding fragment thereof can increase HDL particle size by about 3% to about 10% or by about 3% to about 7%. The increase in HDL particle size can occur within 30 days of the first administration, within 60 days of the first administration, or within 90 days of the first administration.

In some aspects, administration of the anti-EL antibody or antigen-binding fragment thereof increases HDL phospholipids in the subject. The administration of the anti-EL antibody or antigen-binding fragment thereof can increase HDL phospholipids by, for example, at least 50%. The increase in HDL phospholipids can occur within 30 days of the first administration, within 60 days of the first administration, or within 90 days of the first administration.

In some aspects, administration of an anti-EL antibody or antigen-binding fragment thereof increases apolipoprotein A1 (apoA1) in the subject. The administration of the anti-EL antibody or antigen-binding fragment thereof can increase apoA1 by, for example, at least 30%. Thus, the administration of the anti-EL antibody or antigen-binding fragment thereof can increase apoA1 by about 30% to about 40% or by about 30% to about 35%. The increase in apoA1 can occur within 30 days of the first administration, within 60 days of the first administration, or within 90 days of the first administration.

In some aspects, administration of an anti-EL antibody or antigen-binding fragment thereof increases cholesterol efflux capacity (CEC) in the subject. CEC can be measured, e.g., using methods provided in Thacker et al., Journal of Lipid Research 56: 1282-1295 (2015). The administration of the anti-EL antibody or antigen-binding fragment thereof can increase non-ATP-binding cassette transporter A1 (ABCA1) cholesterol efflux capacity by, for example, at least 30% or at least 35%. Thus, the administration of the anti-EL antibody or antigen-binding fragment thereof can increase non-ABCA1 cholesterol efflux capacity by about 30% to about 40% or by about 30% to about 35%.

In some aspects, administration of an anti-EL antibody or antigen-binding fragment thereof increases plasma phosphatidylinositol (PI) levels in the subject. In some aspects, increased PI levels are increased PI(14:2/20:0), PI(14:2/22:0), PI(14:2/22:1), PI(14:2/22:2), PI(16:0/16:1), PI(16:0/18:0), PI(16:0/18:2), PI(16:0/20:2), PI(16:0/20:3), PI(16:0/20:4), PI(16:0/22:4), PI(16:1/18:0), PI(16:1/18:1), PI(18:0/18:0), PI(18:0/18:1), PI(18:0/18:2), PI(18:0/18:3), PI(18:0/20:2), PI(18:0/20:3), PI(18:0/20:4), PI(18:0/22:4), PI(18:0/22:5), PI(18:0/22:6), PI(18:1/16:0), PI(18:1/18:1), PI(18:1/18:2), PI(18:1/20:2), PI(18:1/20:3), PI(18:1/20:4), and/or PI(18:2/18:2) levels. In some aspects, increased PI levels are increased PI(14:2/22:2), PI(16:0/16:1), PI(16:0/18:2), PI(16:0/20:3), PI(16:0/20:4), PI(18:0/18:1), PI(18:0/18:2), PI(18:0/20:2), PI(18:0/20:3), PI(18:0/20:4), PI(18:0/22:6), and/or PI(18:1/16:0) levels. The administration of the anti-EL antibody or antigen-binding fragment thereof can increase plasma PI levels by, for example, at least several hundred percent for more abundant PI species and by multiple hundreds to thousands percent change from baseline for less abundant PI species. Thus, the administration of the anti-EL antibody or antigen-binding fragment thereof can increase plasma PI by about 100-1000% depending on PI species. In some instances, administration of the anti-EL antibody or antibody-binding fragment thereof increases levels of at least 10 plasma PI species e.g., by at least 100% or by 100-1000%. In some instances, administration of the anti-EL antibody or antigen-binding fragment thereof increases levels of at least 12 plasma PI species (e.g., PI(14:2/22:2), PI(16:0/16:1), PI(16:0/18:2), PI(16:0/20:3), PI(16:0/20:4), PI(18:0/18:1), PI(18:0/18:2), PI(18:0/20:2), PI(18:0/20:3), PI(18:0/20:4), PI(18:0/22:6), and PI(18:1/16:0)) e.g., by at least 100% or by 100-1000%. In some instances, administration of the anti-EL antibody or antigen-binding fragment thereof increases levels of at least 30 plasma PI species (e.g., PI(14:2/20:0), PI(14:2/22:0), PI(14:2/22:1), PI(14:2/22:2), PI(16:0/16:1), PI(16:0/18:0), PI(16:0/18:2), PI(16:0/20:2), PI(16:0/20:3), PI(16:0/20:4), PI(16:0/22:4), PI(16:1/18:0), PI(16:1/18:1), PI(18:0/18:0), PI(18:0/18:1), PI(18:0/18:2), PI(18:0/18:3), PI(18:0/20:2), PI(18:0/20:3), PI(18:0/20:4), PI(18:0/22:4), PI(18:0/22:5), PI(18:0/22:6), PI(18:1/16:0), PI(18:1/18:1), PI(18:1/18:2), PI(18:1/20:2), PI(18:1/20:3), PI(18:1/20:4), and PI(18:2/18:2), e.g., by at least 100% or by 100-1000%.

In some instances, administration of the anti-EL antibody or antibody-binding fragment thereof increases levels of at least 10 plasma PI species by at least 250% or by 250-1000%. In some instances, administration of the anti-EL antibody or antigen-binding fragment thereof increases levels of at least 12 plasma PI species (e.g., PI(14:2/22:2), PI(16:0/16:1), PI(16:0/18:2), PI(16:0/20:3), PI(16:0/20:4), PI(18:0/18:1), PI(18:0/18:2), PI(18:0/20:2), PI(18:0/20:3), PI(18:0/20:4), PI(18:0/22:6), and PI(18:1/16:0)) by at least 250% or by 250-1000%. In some instances, administration of the anti-EL antibody or antigen-binding fragment thereof increases levels of at least 30 plasma PI species (e.g., PI(14:2/20:0), PI(14:2/22:0), PI(14:2/22:1), PI(14:2/22:2), PI(16:0/16:1), PI(16:0/18:0), PI(16:0/18:2), PI(16:0/20:2), PI(16:0/20:3), PI(16:0/20:4), PI(16:0/22:4), PI(16:1/18:0), PI(16:1/18:1), PI(18:0/18:0), PI(18:0/18:1), PI(18:0/18:2), PI(18:0/18:3), PI(18:0/20:2), PI(18:0/20:3), PI(18:0/20:4), PI(18:0/22:4), PI(18:0/22:5), PI(18:0/22:6), PI(18:1/16:0), PI(18:1/18:1), PI(18:1/18:2), PI(18:1/20:2), PI(18:1/20:3), PI(18:1/20:4), and PI(18:2/18:2), by at least 250% or by 250-1000%.

The increase in plasma PI can occur within 90 days of the first administration.

In some aspects, an anti-EL antibody or antigen-binding fragment thereof or a pharmaceutical composition thereof is administered at a dose of about 100 mg to about 350 mg. In some aspects, an anti-EL antibody or antigen-binding fragment thereof or a pharmaceutical composition thereof is administered at a dose of about 100 mg to about 250 mg. In some aspects, an anti-EL antibody or antigen-binding fragment thereof or a pharmaceutical composition thereof is administered at a dose of about 100 mg to about 200 mg. The dose of the antibody or antigen-binding fragment thereof or a pharmaceutical composition thereof can be administered parenterally, e.g., subcutaneously. The dose of the antibody or antigen-binding fragment thereof or a pharmaceutical composition thereof can be administered using an accessorized pre-filled syringe (APFS) or an auto-injector. The dose of the antibody or antigen-binding fragment thereof or a pharmaceutical composition thereof can be administered about once a month.

In some aspects, an anti-EL antibody or antigen-binding fragment thereof or a pharmaceutical composition thereof is administered at a dose of about 200 mg to about 350 mg. In some aspects, an anti-EL antibody or antigen-binding fragment thereof or a pharmaceutical composition thereof is administered at a dose of about 200 mg to about 300 mg. In some aspects, an anti-EL antibody or antigen-binding fragment thereof or a pharmaceutical composition thereof is administered at a dose of about 200 mg to about 250 mg. The dose of the antibody or antigen-binding fragment thereof or a pharmaceutical composition thereof can be administered parenterally, e.g., subcutaneously. The dose of the antibody or antigen-binding fragment thereof or a pharmaceutical composition thereof can be administered using an accessorized pre-filled syringe (APFS) or an auto-injector. The dose of the antibody or antigen-binding fragment thereof or a pharmaceutical composition thereof can be administered about once a month.

In some aspects, an anti-EL antibody or antigen-binding fragment thereof or a pharmaceutical composition thereof is administered at a dose of about 250 mg to about 300 mg. In some aspects, an anti-EL antibody or antigen-binding fragment thereof or a pharmaceutical composition thereof is administered at a dose of about 250 mg to about 350 mg. The dose of the antibody or antigen-binding fragment thereof or a pharmaceutical composition thereof can be administered parenterally, e.g., subcutaneously. The dose of the antibody or antigen-binding fragment thereof or a pharmaceutical composition thereof can be administered using an accessorized pre-filled syringe (APFS) or an auto-injector. The dose of the antibody or antigen-binding fragment thereof or a pharmaceutical composition thereof can be administered about once a month.

In some aspects, an anti-EL antibody or antigen-binding fragment thereof or a pharmaceutical composition thereof is administered at a dose of about 100 mg. In some aspects, an anti-EL antibody or antigen-binding fragment thereof or a pharmaceutical composition thereof is administered at a dose of about 110 mg. In some aspects, an anti-EL antibody or antigen-binding fragment thereof or a pharmaceutical composition thereof is administered at a dose of about 120 mg. In some aspects, an anti-EL antibody or antigen-binding fragment thereof or a pharmaceutical composition thereof is administered at a dose of about 125 mg. In some aspects, an anti-EL antibody or antigen-binding fragment thereof or a pharmaceutical composition thereof is administered at a dose of about 130 mg. In some aspects, an anti-EL antibody or antigen-binding fragment thereof or a pharmaceutical composition thereof is administered at a dose of about 140 mg. In some aspects, an anti-EL antibody or antigen-binding fragment thereof or a pharmaceutical composition thereof is administered at a dose of about 150 mg. In some aspects, an anti-EL antibody or antigen-binding fragment thereof or a pharmaceutical composition thereof is administered at a dose of about 160 mg. In some aspects, an anti-EL antibody or antigen-binding fragment thereof or a pharmaceutical composition thereof is administered at a dose of about 170 mg. In some aspects, an anti-EL antibody or antigen-binding fragment thereof or a pharmaceutical composition thereof is administered at a dose of about 175 mg. In some aspects, an anti-EL antibody or antigen-binding fragment thereof or a pharmaceutical composition thereof is administered at a dose of about 180 mg. In some aspects, an anti-EL antibody or antigen-binding fragment thereof or a pharmaceutical composition thereof is administered at a dose of about 190 mg. In some aspects, an anti-EL antibody or antigen-binding fragment thereof or a pharmaceutical composition thereof is administered at a dose of about 200 mg. In some aspects, an anti-EL antibody or antigen-binding fragment thereof or a pharmaceutical composition thereof is administered at a dose of about 210 mg. In some aspects, an anti-EL antibody or antigen-binding fragment thereof or a pharmaceutical composition thereof is administered at a dose of about 220 mg. In some aspects, an anti-EL antibody or antigen-binding fragment thereof or a pharmaceutical composition thereof is administered at a dose of about 225 mg. In some aspects, an anti-EL antibody or antigen-binding fragment thereof or a pharmaceutical composition thereof is administered at a dose of about 230 mg. In some aspects, an anti-EL antibody or antigen-binding fragment thereof or a pharmaceutical composition thereof is administered at a dose of about 240 mg. In some aspects, an anti-EL antibody or antigen-binding fragment thereof or a pharmaceutical composition thereof is administered at a dose of about 250 mg. In some aspects, an anti-EL antibody or antigen-binding fragment thereof or a pharmaceutical composition thereof is administered at a dose of about 260 mg. In some aspects, an anti-EL antibody or antigen-binding fragment thereof or a pharmaceutical composition thereof is administered at a dose of about 270 mg. In some aspects, an anti-EL antibody or antigen-binding fragment thereof or a pharmaceutical composition thereof is administered at a dose of about 275 mg. In some aspects, an anti-EL antibody or antigen-binding fragment thereof or a pharmaceutical composition thereof is administered at a dose of about 280 mg. In some aspects, an anti-EL antibody or antigen-binding fragment thereof or a pharmaceutical composition thereof is administered at a dose of about 290 mg. In some aspects, an anti-EL antibody or antigen-binding fragment thereof or a pharmaceutical composition thereof is administered at a dose of about 300 mg. In some aspects, an anti-EL antibody or antigen-binding fragment thereof or a pharmaceutical composition thereof is administered at a dose of about 310 mg. In some aspects, an anti-EL antibody or antigen-binding fragment thereof or a pharmaceutical composition thereof is administered at a dose of about 320 mg. In some aspects, an anti-EL antibody or antigen-binding fragment thereof or a pharmaceutical composition thereof is administered at a dose of about 325 mg. In some aspects, an anti-EL antibody or antigen-binding fragment thereof or a pharmaceutical composition thereof is administered at a dose of about 330 mg. In some aspects, an anti-EL antibody or antigen-binding fragment thereof or a pharmaceutical composition thereof is administered at a dose of about 340 mg. In some aspects, an anti-EL antibody or antigen-binding fragment thereof or a pharmaceutical composition thereof is administered at a dose of about 350 mg. The dose of the antibody or antigen-binding fragment thereof or a pharmaceutical composition thereof can be administered parenterally, e.g., subcutaneously. The dose of the antibody or antigen-binding fragment thereof or a pharmaceutical composition thereof can be administered using an accessorized pre-filled syringe (APFS) or an auto-injector. The dose of the antibody or antigen-binding fragment thereof or a pharmaceutical composition thereof can be administered about once a month.

In some aspects, an anti-EL antibody or antigen-binding fragment thereof or a pharmaceutical composition thereof is administered at a dose of about 125 mg or 125 mg. The about 125 mg or the 125 mg dose of the antibody or antigen-binding fragment thereof or a pharmaceutical composition thereof can be administered parenterally, e.g., subcutaneously. The about 125 mg or the 125 mg dose of the antibody or antigen-binding fragment thereof or a pharmaceutical composition thereof can be administered using an accessorized pre-filled syringe (APFS) or an auto-injector.

In some aspects, an anti-EL antibody or antigen-binding fragment thereof or a pharmaceutical composition thereof is administered at a dose of about 250 mg or 250 mg. The about 250 mg or the 250 mg dose of the antibody or antigen-binding fragment thereof or a pharmaceutical composition thereof can be administered parenterally, e.g., subcutaneously. The about 250 mg or the 250 mg dose of the antibody or antigen-binding fragment thereof or a pharmaceutical composition thereof can be administered using an accessorized pre-filled syringe (APFS) or an auto-injector.

In some aspects, an anti-EL antibody or antigen-binding fragment thereof or a pharmaceutical composition thereof is administered about once a month or once a month. The antibody or antigen-binding fragment thereof or a pharmaceutical composition thereof that is administered about once a month or once a month can be administered parenterally, e.g., subcutaneously. The antibody or antigen-binding fragment thereof or a pharmaceutical composition thereof that is administered about once a month or once a month can be administered using an accessorized pre-filled syringe (APFS) or an auto-injector.

In some aspects, an anti-EL antibody or antigen-binding fragment thereof or a pharmaceutical composition thereof is administered at a dose of about 125 mg about once a month. The about 125 mg dose administered about once a month can be administered parenterally, e.g., subcutaneously. The about 125 mg dose administered about once a month can be administered using an accessorized pre-filled syringe (APFS) or an auto-injector.

In some aspects, an anti-EL antibody or antigen-binding fragment thereof or a pharmaceutical composition thereof is administered at a dose of about 250 mg about once a month. The about 250 mg dose administered about once a month can be administered parenterally, e.g., subcutaneously. The about 250 mg dose administered about once a month can be administered using an accessorized pre-filled syringe (APFS) or an auto-injector.

In some aspects, an anti-EL antibody or antigen-binding fragment thereof or a pharmaceutical composition thereof is administered at a dose of 125 mg once a month. The 125 mg dose administered once a month can be administered parenterally, e.g., subcutaneously. The about 125 mg dose administered about once a month can be administered using an accessorized pre-filled syringe (APFS) or an auto-injector.

In some aspects, an anti-EL antibody or antigen-binding fragment thereof or a pharmaceutical composition thereof is administered at a dose of 250 mg once a month. The 250 mg dose administered once a month can be administered parenterally, e.g., subcutaneously. The about 250 mg dose administered about once a month can be administered using an accessorized pre-filled syringe (APFS) or an auto-injector.

According to the methods provided herein, the anti-EL antibody or antigen binding fragment thereof, or the pharmaceutical composition comprising anti-EL antibodies or antigen-binding fragments thereof, can be administered parenterally. In a some aspects of the present disclosure, the anti-EL antibody or antigen binding fragment thereof, or the pharmaceutical composition comprising anti-EL antibodies or antigen-binding fragments thereof, is administered subcutaneously.

In some aspects of the present disclosure, the anti-EL antibodies or antigen-binding fragments thereof are administered (e.g., about once a month) for at least 3 months. In some aspects of the present disclosure, the anti-EL antibodies or antigen-binding fragments thereof are administered (e.g., about once a month) for at least 12 months. In some aspects of the present disclosure, the anti-EL antibodies or antigen-binding fragments thereof are administered (e.g., about once a month) for at least 24 months.

In some aspects of the present disclosure, the present disclosure relates to an anti-EL antibody or antigen-binding fragment thereof or pharmaceutical composition provided herein for use as a medicament, wherein the medicament is for administration at about 100 mg to about 350 mg or about 200 mg to about 350 mg (e.g., 250 mg). In some aspects of the present disclosure, the present disclosure relates to an anti-EL antibody or antigen-binding fragment thereof or pharmaceutical composition provided herein for use as a medicament, wherein the medicament is for administration at about 100 mg to about 350 mg or about 200 mg to about 350 mg (e.g., about 250 mg).

In some aspects of the present disclosure, the present disclosure relates to an anti-EL antibody or antigen-binding fragment thereof or pharmaceutical composition provided herein for use as a medicament, wherein the medicament is for administration at about 100 mg to about 350 mg or about 200 mg to about 350 mg (e.g., 250 mg) about once a month. In some aspects of the present disclosure, the present disclosure relates to an anti-EL antibody or antigen-binding fragment thereof or pharmaceutical composition provided herein for use as a medicament, wherein the medicament is for administration at about 100 mg to about 350 mg or about 200 mg to about 350 mg (e.g., about 250 mg) once a month.

According to the methods provided herein, the anti-EL antibody or antigen binding fragment thereof, or the pharmaceutical composition comprising anti-EL antibodies or antigen-binding fragments thereof, can be administered in combination with an inhibitor of PCSK9. Inhibitors of PCSK9 are disclosed, for example, in Chaudhary et al., World J. Cardiol. 9: 76-91 (2017) and in Chodorge et al., Sci. Rep. 8: 17545 (2018), each of which is herein incorporated by reference. Inhibitors of PCSK9 can be, for example, antibodies or antigen-binding fragments that bind to PCSK9. Antibodies or antigen-binding fragments thereof that inhibit PCSK9 include, for example, HS9, alirocumab, evolocumab, and bococizumab.

The HS9 antibody (in the context of a GLP-1 fusion protein called MEDI4166) is disclosed in Chodorge et al., Sci. Rep. 8: 17545 (2018) and International PCT Publication No. WO2015127273, each of which is herein incorporated by reference in its entirety. The HS9 antibody comprises the following variable heavy and variable light chain sequences:

HS9 variable heavy chain sequence:
(SEQ ID NO: 14)
QVQLVQSGAEVKKPGASVKVSCKASGYTFTSYYMH
WVRQAPGQGLEWMGEISPSGGSTSYNQKFQGRVTM
TRDTSTSTVYMELSSLRSEDTAVYYCARERPLYAS
DLWGQGTTVTVSS
HS9 variable light chain sequence:
(SEQ ID NO: 15)
DIQMTQSPSSLSASVGDRVTITCQASQDVKTAVAW
YQQKPGKAPKLLIYSASYRYTGVPSRFSGSGSGTD
FTFTISSLQPEDIATYYCQQRYSLWRTFGQGTKLE
IK.

In some aspects of the present disclosure, an antibody or antigen binding fragment thereof that inhibits PCSK9 comprises a variable heavy chain sequence of SEQ ID NO:14. In some aspects of the present disclosure, an antibody or antigen binding fragment thereof that inhibits PCSK9 comprises a variable light chain sequence of SEQ ID NO:15. In some aspects of the present disclosure, an antibody or antigen binding fragment thereof that inhibits PCSK9 comprises a variable heavy chain sequence of SEQ ID NO:14 and a variable light chain sequence of SEQ ID NO:15.

In some aspects of the present disclosure, an antibody that inhibits PCSK9 comprises a human IgG1 heavy chain. In some aspects of the present disclosure, an antibody that inhibits PCSK9 comprises a human IgG1 heavy chain containing the triple mutation L234F/L235E/P331S (“IgG1-TM”). In some aspects of the present disclosure, an antibody that inhibits PCSK9 comprises a human kappa light chain. In some aspects of the present disclosure, an antibody that inhibits PCSK9 comprises a) an IgG1-TM heavy chain comprising a variable heavy chain sequence of SEQ ID NO:14, and b) a kappa light chain comprising a variable light chain sequence of SEQ ID NO:15.

In some aspects of the present disclosure, the inhibitor of PCSK9 is capable of promoting LDL-C uptake in HepG2 cells treated with recombinant PCSK9 (e.g. as disclosed in Chodorge et al., Sci. Rep. 8: 17545 (2018)).

As provided herein, the inhibitor of PCSK9 can be administered simultaneously (in the same or separate pharmaceutical compositions) or sequentially with the anti-EL antibody or antigen binding fragment thereof.

### 5.3 EL Antibodies and Antigen-Binding Fragments Thereof

Provided herein are methods of treating cardiovascular disease in a subject (e.g., a human subject) comprising administering to the subject antibodies (e.g., monoclonal antibodies, such as chimeric, humanized, or human antibodies) and antigen-binding fragments thereof which specifically bind to EL (e.g., human EL). Exemplary EL antibodies and antigen-binding fragments thereof that can be used in the methods provided herein are known in the art. The amino acid sequences for human EL is known in the art and the mature version of the protein (lacking the leader sequence) is provided herein as the sequence of SEQ ID NO:11.

Mature Human EL (lacking leader sequence):
(SEQ ID NO: 11)
SPVPFGPEGRLEDKLHKPKATQTEVKPSVRFNLRT
SKDPEHEGCYLSVGHSQPLEDCSFNMTAKTFFIIH
GWTMSGIFENWLHKLVSALHTREKDANVVVVDWLP
LAHQLYTDAVNNTRVVGHSIARMLDWLQEKDDFSL
GNVHLIGYSLGAHVAGYAGNFVKGTVGRITGLDPA
GPMFEGADIHKRLSPDDADFVDVLHTYTRSFGLSI
GIQMPVGHIDIYPNGGDFQPGCGLNDVLGSIAYGT
ITEVVKCEHERAVHLFVDSLVNQDKPSFAFQCTDS
NRFKKGICLSCRKNRCNSIGYNAKKMRNKRNSKMY
LKTRAGMPFRVYHYQMKIHVFSYKNMGEIEPTFYV
TLYGTNADSQTLPLEIVERIEQNATNTFLVYTEED
LGDLLKIQLTWEGASQSWYNLWKEFRSYLSQPRNP
GRELNIRRIRVKSGETQRKLTFCTEDPENTSISPG
RELWFRKCRDGWRMKNETSPTVELP.

In some aspects of the present disclosure, an antibody or antigen-binding fragment thereof for use in the methods described herein specifically binds to human EL. In some aspects of the present disclosure, an antibody or antigen-binding fragment thereof for use in the methods described herein specifically binds to human and cynomolgus monkey EL.

In some aspects of the present disclosure, the anti-EL antibody or antigen-binding fragment thereof binds to human EL with an equilibrium dissociation constant (KD) of about 4.06 nM (e.g., as measured using surface plasmon resonance). In some aspects of the present disclosure, the anti-EL antibody or antigen-binding fragment thereof binds to cynomolgus monkey EL with a KD of about 1.56 nM (e.g., as measured using surface plasmon resonance). In some aspects of the present disclosure, the anti-EL antibody or antigen-binding fragment thereof binds to human EL with a KD constant of about 4.06 nM and to cynomolgus monkey EL with a KD of about 1.56 nM (e.g., as measured using surface plasmon resonance).

In some aspects of the present disclosure, the anti-EL antibody or antigen-binding fragment thereof is capable of neutralizing or inhibiting EL.

The ability of an anti-EL antibody or antigen-binding fragment thereof to neutralize EL can be determined using the following protocol: conditioned medium are incubated in half-area 96-well microplates with assay buffer (20 mM Tris-HCl, 150 mM NaCl, 4 mM CaCl2, 0.5% BSA) and HDL (e.g., human HDL) in the presence or absence of the anti-EL antibody or antigen-binding fragment thereof at concentrations ranging from 1000 nM-31.6 pM for two hours at 37° C. Following this incubation, free fatty acid release is measured in each well using the NEFA-HR(2) assay kit (Wako Diagnostics, Mountain View, Calif.). Absorbance at both 550 nm and 660 nm are measured using a SpectraMax M5 plate reader. The value obtained from subtracting the 660 nm reading from the 550 nm reading is used for further analysis. The percent activity “Ax” at an antibody concentration of “x” is calculated using the following equation:

Ax=[(Ex−V0)/(E0−V0)]×100

where “Ex” is the mean values of absorbance unit with an inhibitor in the presence of an enzyme, and “E0” and “V0” are the mean values of absorbance unit without an inhibitor, and in the absence of an enzyme, respectively. The 50% inhibitory concentrations (IC50) are calculated and plotted using GraphPad Prism.

The same assay can be performed using VLDL (e.g., human VLDL) as the substrate instead of HDL (e.g., human HDL) to show that an anti-EL antibody does not neutralize hepatic lipase or lipoprotein lipase.

In some aspects of the present disclosure, the anti-EL antibody or antigen-binding fragment thereof has a half maximal inhibitory concentration (IC₅₀) of about 1.3 nM for human EL. In some aspects of the present disclosure, the anti-EL antibody or antigen-binding fragment thereof has an IC₅₀ of about 1.7 nM for cynomolgus monkey EL. In some aspects of the present disclosure, the anti-EL antibody or antigen-binding fragment thereof has an IC₅₀ of about 1.3 nM for human EL and an IC₅₀ of about 1.7 nM for cynomolgus monkey EL.

In some aspects of the present disclosure, the anti-EL antibody or antigen-binding fragment thereof does not inhibit very low-density lipoprotein (VLDL) lipolysis mediated by either hepatic lipase or lipoprotein lipase.

In some aspects of the present disclosure, the anti-EL antibody or antigen-binding fragment thereof does not block the exchange of cholesterol from HDL to LDL particles.

In some aspects of the present disclosure, the anti-EL antibody or antigen-binding fragment thereof increases delivery of low-density lipoprotein (LDL) to low-density lipoprotein receptor (LDLR).

In some aspects of the present disclosure, an antibody or antigen-binding fragment thereof for use in the methods described herein specifically binds to human EL and comprises the six CDRs of the MEDI5884 antibody listed as provided in Tables 1 and 2.

TABLE 1
VH CDR Amino Acid Sequences ¹

| | VH CDR1 | VH CDR2 | VH CDR3 |
|---|---|---|---|
| Anti- | (SEQ ID | (SEQ ID | (SEQ ID |
| body | NO:) | NO:) | NO:) |
| MEDI5 | NYALN | WINTYSGVGT | RGFYGRR |
| 884 | (SEQ ID | YAGEFKG | FFDV |
| | NO: 1) | (SEQ ID NO: 2) | (SEQ ID NO: 3) |

¹ The VH CDRs in Table 1 are determined according to Kabat.

TABLE 2
VL CDR Amino Acid Sequences ²

| | VL CDR1 | VL CDR2 | VL CDR3 |
|---|---|---|---|
| Antibody | (SEQ ID NO:) | (SEQ ID NO:) | (SEQ ID NO:) |
| MEDI5884 | KASQSVD | AASNLAS | QQTIEDPPT |
| | YDVDSYMH | (SEQ ID | (SEQ ID |
| | (SEQ ID NO: 4) | NO: 5) | NO: 6) |

² The VL CDRs in Table 2 are determined according to Kabat.

In some aspects of the present disclosure, an antibody or antigen-binding fragment thereof for use in the methods described herein specifically binds to human EL and comprises the VH of the MEDI5884 antibody listed in Table 3.

TABLE 3
Variable Heavy Chain (VH) Amino Acid Sequences

| Antibody | VH Amino Acid Sequence (SEQ ID NO) |
|---|---|
| MEDI5884 | QVQLVQSGSELKKPGASVKVSCKASGYTFTN |
| | YALNWVRQAPGQGLEWMGWINTYSGVGTYAG |
| | EFKGRFVFSLDTSVSTAYLQISSLKAEDTAV |
| | YYCARRGFYGRRFFDVWGKGTTVTVSS |
| | (SEQ ID NO: 7) |

In some aspects of the present disclosure, an antibody or antigen-binding fragment thereof for use in the methods described herein specifically binds to human EL and comprises the VL of the MEDI5884 antibody listed in Table 4.

TABLE 4
Variable Light Chain (VL) Amino Acid Sequences

| Antibody | VL Amino Acid Sequence (SEQ ID NO) |
|---|---|
| MEDI5884 | DIQLTQSPSSLSASVGDRVTITCKASQSVDYDV |
| | DSYMHWYQQKPGKAPKLLIYAASNLASGVPSRF |
| | SGSGSGTDFTFTISSLQPEDIATYYCQQTI |
| | EDPPTFGGGTKVEIK (SEQ ID NO: 8) |

In some aspects of the present disclosure, an antibody or antigen-binding fragment thereof for use in the methods described herein specifically binds to human EL and comprises the VH and the VL of the MEDI5884 antibody listed in Tables 3 and 4.

In some aspects of the present disclosure, an antibody or antigen-binding fragment thereof for use in the methods described herein specifically binds to human EL and comprises the heavy chain sequence of the MEDI5884 antibody listed in Table 5.

TABLE 5
Full-length heavy chain amino acid sequences

| | Full-Length Heavy Chain Amino |
|---|---|
| Antibody | Acid Sequence (SEQ ID NO) |
| MEDI5884 | QVQLVQSGSELKKPGASVKVSCKASGYTFTNYALNW |
| | VRQAPGQGLEWMGWINTYSGVGTYAGEFKGRFVFS |
| | LDTSVSTAYLQISSLKAEDTAVYYCARRGFYGRRF |
| | FDVWGKGTTVTVSSASTKGPSVFPLAPCSRSTSES |
| | TAALGCLVKDYFPEPVTVSWNSGALTSGVHTFPAV |
| | LQSSGLYSLSSWTVPSSSLGTKTYTCNVDHKPSNT |
| | KVDKRVESKYGPPCPPCPAPEFLGGPSVFLFPPKP |
| | KDTLMISRTPEVTCVVVDVSQEDPEVQFNWYVDGV |
| | EVHNAKTKPREEQFNSTYRVVSVLTVLHQDWLNGK |
| | EYKCKVSNKGLPSSIEKTISKAKGQPREPQVYTLP |
| | PSQEEMTKNQVSLTCLVKGFYPSDIAVEWESNGQP |
| | ENNYKTTPPVLDSDGSFFLYSRLTVDKSRWQEGNV |
| | FSCSVMHEALHNHYTQKSLSLSLGK |
| | (SEQ ID NO: 9) |

In some aspects of the present disclosure, an antibody or antigen-binding fragment thereof for use in the methods described herein specifically binds to human EL and comprises the light chain sequence of the MEDI5884 antibody listed in Table 6.

TABLE 6
Full-length light chain amino acid sequences

| | Full-Length Light Chain Amino |
|---|---|
| Antibody | Acid Sequence (SEQ ID NO) |
| MEDI5884 | DIQLTQSPSSLSASVGDRVTITCKASQSVDYD |
| | VDSYMHWYQQKPGKAPKLLIYAASNLASGVPS |
| | RFSGSGSGTDFTFTISSLQPEDIATYYCQQTI |
| | EDPPTFGGGTKVEIKRTVAAPSVFIFPPSDEQ |
| | LKSGTASVVCLLNNFYPREAKVQWKVDNALQS |
| | GNSQESVTEQDSKDSTYSLSSTLTLSKADYE |
| | KHKVYACEVTHQGLSSPVTKSFNRGEC |
| | (SEQ ID NO: 10) |

In some aspects of the present disclosure, an antibody or antigen-binding fragment for use in the methods described herein specifically binds to human EL and comprises the heavy chain sequence and the light chain sequence of the MEDI5884 antibody listed in Tables 5 and 6.

In some aspects of the present disclosure, an antibody or antigen-binding fragment thereof for use in the methods described herein is described by its VL domain alone, or its VH domain alone, or by its 3 VL CDRs alone, or its 3 VH CDRs alone. See, for example, Rader C et al., (1998) PNAS 95: 8910-8915, which is incorporated herein by reference in its entirety, describing the humanization of the mouse anti-αvβ3 antibody by identifying a complementing light chain or heavy chain, respectively, from a human light chain or heavy chain library, resulting in humanized antibody variants having affinities as high or higher than the affinity of the original antibody. See also Clackson T et al., (1991) Nature 352: 624-628, which is incorporated herein by reference in its entirety, describing methods of producing antibodies that specifically bind a specific antigen by using a specific VL domain (or VH domain) and screening a library for the complementary VH domain or (VL domain). The screen produced 14 new partners for a specific VH domain and 13 new partners for a specific VL domain, which were strong binders, as determined by ELISA. See also Kim S J & Hong H J, (2007) J Microbiol 45: 572-577, which is incorporated herein by reference in its entirety, describing methods of producing antibodies that specifically bind a specific antigen by using a specific VH domain and screening a library (e.g., human VL library) for complementary VL domains; the selected VL domains in turn could be used to guide selection of additional complementary (e.g., human) VH domains.

In some aspects, the CDRs of an antibody or antigen-binding fragment thereof can be determined according to the Chothia numbering scheme, which refers to the location of immunoglobulin structural loops (see, e.g., Chothia C & Lesk A M, (1987), J Mol Biol 196: 901-917; Al-Lazikani B et al., (1997) J Mol Biol 273: 927-948; Chothia C et al., (1992) J Mol Biol 227: 799-817; Tramontano A et al., (1990) J Mol Biol 215(1): 175-82; and U.S. Pat. No. 7,709,226). Typically, when using the Kabat numbering convention, the Chothia CDR-H1 loop is present at heavy chain amino acids 26 to 32, 33, or 34, the Chothia CDR-H2 loop is present at heavy chain amino acids 52 to 56, and the Chothia CDR-H3 loop is present at heavy chain amino acids 95 to 102, while the Chothia CDR-L1 loop is present at light chain amino acids 24 to 34, the Chothia CDR-L2 loop is present at light chain amino acids 50 to 56, and the Chothia CDR-L3 loop is present at light chain amino acids 89 to 97. The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34).

In some aspects, provided herein are methods of administering antibodies and antigen-binding fragments thereof that specifically bind to EL (e.g., human EL) and comprise the Chothia VH and VL CDRs of the MEDI5884 antibody listed in Tables 3 and 4. In some aspects of the present disclosure, provided herein are methods of administering antibodies or antigen-binding fragments thereof that specifically bind to EL (e.g., human EL) and comprise one or more CDRs, in which the Chothia and Kabat CDRs have the same amino acid sequence. In some aspects of the present disclosure, provided herein are methods of administering antibodies and antigen-binding fragments thereof that specifically bind to EL (e.g., human EL) and comprise combinations of Kabat CDRs and Chothia CDRs.

In some aspects of the present disclosure, the CDRs of an antibody or antigen-binding fragment thereof can be determined according to the IMGT numbering system as described in Lefranc M-P, (1999) The Immunologist 7: 132-136 and Lefranc M-P et al., (1999) Nucleic Acids Res 27: 209-212. According to the IMGT numbering scheme, VH-CDR1 is at positions 26 to 35, VH-CDR2 is at positions 51 to 57, VH-CDR3 is at positions 93 to 102, VL-CDR1 is at positions 27 to 32, VL-CDR2 is at positions 50 to 52, and VL-CDR3 is at positions 89 to 97. In some aspects of the present disclosure, provided herein are methods of administering antibodies and antigen-binding fragments thereof that specifically bind to EL (e.g., human EL) and comprise the IMGT VH and VL CDRs of the MEDI5884 antibody listed in Tables 3 and 4, for example, as described in Lefranc M-P (1999) supra and Lefranc M-P et al., (1999) supra).

In some aspects, the CDRs of an antibody or antigen-binding fragment thereof can be determined according to MacCallum R M et al., (1996) J Mol Biol 262: 732-745. See also, e.g., Martin A. “Protein Sequence and Structure Analysis of Antibody Variable Domains,” in Antibody Engineering, Kontermann and Dübel, eds., Chapter 31, pp. 422-439, Springer-Verlag, Berlin (2001). In some aspects of the present disclosure, provided herein are methods of administering antibodies or antigen-binding fragments thereof that specifically bind to EL (e.g., human EL) and comprise VH and VL CDRs of the MEDI5884 antibody listed in Tables 3 and 4 as determined by the method in MacCallum R M et al.

In some aspects, the CDRs of an antibody or antigen-binding fragment thereof can be determined according to the AbM numbering scheme, which refers AbM hypervariable regions which represent a compromise between the Kabat CDRs and Chothia structural loops, and are used by Oxford Molecular's AbM antibody modeling software (Oxford Molecular Group, Inc.). In some aspects of the present disclosure, provided herein are methods of administering antibodies or antigen-binding fragments thereof that specifically bind to EL (e.g., human EL) and comprise VH and VL CDRs of the MEDI5884 antibody listed in Tables 3 and 4 as determined by the AbM numbering scheme.

In some aspects of the present disclosure, provided herein are methods of administering antibodies that comprise a heavy chain and a light chain.

With respect to the heavy chain, in some aspects of the present disclosure, the heavy chain is a gamma heavy chain. The constant region of a human IgG4P heavy chain can comprise the following amino acid sequence:

(SEQ ID NO: 12)
ASTKGPSVFPLAPCSRSTSESTAALGCLVKDYFPEPVTVSWN
SGALTSGVHTFPAVLQSSGLYSLSSVVTVPSSSLGTKTYTCN
VDHKPSNTKVDKRVESKYGPPCPPCPAPEFLGGPSVFLFPPK
PKDTLMISRTPEVTCVVVDVSQEDPEVQFNWYVDGVEVHNAK
TKPREEQFNSTYRVVSVLTVLHQDWLNGKEYKCKVSNKGLPS
SIEKTISKAKGQPREPQVYTLPPSQEEMTKNQVSLTCLVKGF
YPSDIAVEWESNGQPENNYKTTPPVLDSDGSFFLYSRLTVDK
SRWQEGNVFSCSVMHEALHNHYTQKSLSLSLGK.

In some aspects of the present disclosure, an antibody which immunospecifically binds to EL (e.g., human EL) for use in the methods described herein comprises a heavy chain wherein the amino acid sequence of the VH domain comprises the CDR amino acid sequences set forth in Table 1 and wherein the constant region of the heavy chain comprises the amino acid sequence of a human gamma (γ) heavy chain constant region, e.g., human IgG4P.

In some aspects of the present disclosure, an antibody which immunospecifically binds to EL (e.g., human EL) for use in the methods described herein comprises a heavy chain wherein the amino acid sequence of the VH domain comprises the amino acid sequence set forth in Table 3 and wherein the constant region of the heavy chain comprises the amino acid sequence of a human gamma (γ) heavy chain constant region, e.g., human IgG4P.

With respect to the light chain, in some aspects of the present disclosure, the light chain of an antibody described herein is a kappa light chain. The constant region of a human C kappa light chain can comprise the following amino acid sequence:

(SEQ ID NO: 13)
RTVAAPSVFIFPPSDEQLKSGTASVVCLLNNFYPREAKVQWK
VDNALQSGNSQESVTEQDSKDSTYSLSSTLTLSKADYEKHKV
YACEVTHQGLSSPVTKSFNRGEC.

In some aspects of the present disclosure, an antibody which immunospecifically binds to EL (e.g., human EL) for use in the methods described herein comprises a light chain wherein the amino acid sequence of the VL domain comprises the CDR amino acid sequences set forth in Table 2, and wherein the constant region of the light chain comprises the amino acid sequence of a human C kappa light chain constant region.

In some aspects of the present disclosure, an antibody which immunospecifically binds to EL (e.g., human EL) for use in the methods described herein comprises a light chain wherein the amino acid sequence of the VL domain comprises the sequence set forth in Table 4, and wherein the constant region of the light chain comprises the amino acid sequence of a human C kappa light chain constant region.

In some aspects of the present disclosure, an antibody which immunospecifically binds to EL (e.g., human EL) for use in the methods described herein comprises a VH domain and a VL domain comprising an amino acid sequence of any VH and VL domain described herein, and wherein the constant regions comprise the amino acid sequences of the constant regions of an IgG (e.g., a human IgG) immunoglobulin molecule. In some aspects of the present disclosure, an antibody which immunospecifically binds to EL (e.g., human EL) for use in the methods described herein comprises a VH domain and a VL domain comprising an amino acid sequence of any VH and VL domain described herein, and wherein the constant regions comprise the amino acid sequences of the constant regions of an IgG4P kappa (e.g. human IgG4P kappa) immunoglobulin molecule.

As provided herein, an or antigen-binding fragment thereof that immunospecifically binds to EL (e.g., human EL) for use in the methods described herein can have reduced effector function, e.g., as compared to an antibody or antigen-binding fragment thereof with a wild-type IgG1 sequence. The reduced effector function can be, e.g., as a result of the sequence of a constant region of the antibody or antigen-binding fragment thereof.

As provided herein, an antibody or antigen-binding fragment thereof that immunospecifically binds to EL (e.g., human EL) for use in the methods described herein can lack CDC and/or ADCC activity, e.g., as a result of the sequence of the constant region.

In some aspects of the present disclosure, an antibody or antigen-binding fragment thereof described herein, which immunospecifically binds to EL (e.g., human EL), comprises a heavy chain and a light chain, wherein (i) the heavy chain comprises a VH domain comprising the VH CDR1, VL CDR2, and VL CDR3 amino acid sequences of the MEDI5884 antibody listed in Table 1; (ii) the light chain comprises a VL domain comprising the VL CDR1, VH CDR2, and VH CDR3 amino acid sequences of the MEDI5884 antibody listed in Table 2; (iii) the heavy chain further comprises a constant heavy chain domain comprising the amino acid sequence of the constant domain of a human IgG4P heavy chain; and (iv) the light chain further comprises a constant light chain domain comprising the amino acid sequence of the constant domain of a human kappa light chain.

In some aspects of the present disclosure, an antibody or antigen-binding fragment thereof described herein, which immunospecifically binds to EL (e.g., human EL), comprises a heavy chain and a light chain, wherein (i) the heavy chain comprises a VH domain comprising the amino acid sequence of the VH domain of the MEDI5884 antibody listed in Table 3; (ii) the light chain comprises a VL domain comprising the amino acid sequence of the VL domain of the MEDI5884 antibody listed in Table 4; (iii) the heavy chain further comprises a constant heavy chain domain comprising the amino acid sequence of the constant domain of a human IgG4P heavy chain; and (iv) the light chain further comprises a constant light chain domain comprising the amino acid sequence of the constant domain of a human kappa light chain.

In a specific aspect, an antigen-binding fragment as described herein, which immunospecifically binds to EL (e.g., human EL), is selected from the group consisting of a Fab, Fab′, F(ab′)₂, and scFv, wherein the Fab, Fab′, F(ab′)₂, or scFv comprises a heavy chain variable region sequence and a light chain variable region sequence of an anti-EL antibody or antigen-binding fragment thereof as described herein. A Fab, Fab′, F(ab′)2, or scFv can be produced by any technique known to those of skill in the art. In some aspects of the present disclosure, the Fab, Fab′, F(ab′)₂, or scFv further comprises a moiety that extends the half-life of the antibody in vivo. The moiety is also termed a “half-life extending moiety.” Any moiety known to those of skill in the art for extending the half-life of a Fab, Fab′, F(ab′)₂, or scFv in vivo can be used. For example, the half-life extending moiety can include a Fc region, a polymer, an albumin, or an albumin binding protein or compound. The polymer can include a natural or synthetic, optionally substituted straight or branched chain polyalkylene, polyalkenylene, polyoxylalkylene, polysaccharide, polyethylene glycol, polypropylene glycol, polyvinyl alcohol, methoxypolyethylene glycol, lactose, amylose, dextran, glycogen, or derivative thereof. Substituents can include one or more hydroxy, methyl, or methoxy groups. In some aspects of the present disclosure, the Fab, Fab′, F(ab′)₂, or scFv can be modified by the addition of one or more C-terminal amino acids for attachment of the half-life extending moiety. In some aspects of the present disclosure the half-life extending moiety is polyethylene glycol or human serum albumin. In some aspects of the present disclosure, the Fab, Fab′, F(ab′)₂, or scFv is fused to an Fc region.

### 5.4 Pharmaceutical Compositions

Provided herein are methods of administering compositions comprising an anti-EL antibody or antigen-binding fragment thereof having the desired degree of purity in a physiologically acceptable carrier, excipient, or stabilizer (Remington's Pharmaceutical Sciences (1990) Mack Publishing Co., Easton, Pa.). Acceptable carriers, excipients, or stabilizers are nontoxic to recipients at the dosages and concentrations employed. (See, e.g., Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7th ed., Lippencott Williams and Wilkins (2004); Kibbe et al., Handbook of Pharmaceutical Excipients, 3rd ed., Pharmaceutical Press (2000)). The compositions to be used for in vivo administration can be sterile. This is readily accomplished by filtration through, e.g., sterile filtration membranes.

In some aspects of the present disclosure, methods of administering a pharmaceutical composition are provided, wherein the pharmaceutical composition comprises (i) an isolated antibody or antigen-binding fragment thereof that specifically binds to human EL, comprising (a) the heavy chain variable region (VH) complementarity determining region (CDR) 1, VH CDR2, VH CDR3 and light chain variable region (VL) CDR1, CDR2, and CDR3 sequences of SEQ ID NOs:1-6, respectively, (b) a variable heavy chain region comprising the amino acid sequence of SEQ ID NO:7 and/or a variable light chain region comprising the amino acid sequence of SEQ ID NO:8, or (c) a heavy chain comprising the amino acid sequence of SEQ ID NO:9 and/or a light chain comprising the amino acid sequence of SEQ ID NO:10, and (ii) a pharmaceutically acceptable excipient.

In some aspects of the present disclosure, a pharmaceutical composition comprising an isolated antibody or antigen-binding fragment thereof that specifically binds to human EL also comprises an inhibitor of PCSK9. In some aspects of the present disclosure, a pharmaceutical composition comprising an isolated antibody or antigen-binding fragment thereof that specifically binds to human EL is for administration in combination with an inhibitor of PCSK9.

### 5.5 Antibody Production and Polynucleotides

Antibodies and antigen-binding fragments thereof that immunospecifically bind to EL (e.g., human EL) can be produced by any method known in the art for the synthesis of antibodies and antigen-binding fragments thereof, for example, by chemical synthesis or by recombinant expression techniques. The methods described herein employ, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art. These techniques are described, for example, in the references cited herein and are fully explained in the literature. See, e.g., Sambrook J et al., (2001) Molecular Cloning: A Laboratory Manual, Cold Spring Harbor Laboratory Press, Cold Spring Harbor, N.Y.; Ausubel F M et al., Current Protocols in Molecular Biology, John Wiley & Sons (1987 and annual updates); Current Protocols in Immunology, John Wiley & Sons (1987 and annual updates) Gait (ed.) (1984) Oligonucleotide Synthesis: A Practical Approach, IRL Press; Eckstein (ed.) (1991) Oligonucleotides and Analogues: A Practical Approach, IRL Press; Birren B et al., (eds.) (1999) Genome Analysis: A Laboratory Manual, Cold Spring Harbor Laboratory Press.

In some aspects, provided herein are methods of administering an anti-EL antibody or antigen-binding fragment thereof or a pharmaceutical composition comprising such antibodies or fragments, wherein the antibodies or fragments are produced by recombinant expression of a polynucleotide comprising a nucleotide sequence in a host cell.

In some aspects, the anti-EL antibodies or antigen-binding fragments administered according to the methods provided herein are encoded by polynucleotides encoding anti-EL antibodies or antigen-binding fragments thereof or a domain thereof that are optimized, e.g., by codon/RNA optimization, replacement with heterologous signal sequences, and elimination of mRNA instability elements. Methods to generate optimized nucleic acids encoding an anti-EL antibody or antigen-binding fragment thereof or a domain thereof (e.g., heavy chain, light chain, VH domain, or VL domain) for recombinant expression by introducing codon changes (e.g., a codon change that encodes the same amino acid due to the degeneracy of the genetic code) and/or eliminating inhibitory regions in the mRNA can be carried out by adapting the optimization methods described in, e.g., U.S. Pat. Nos. 5,965,726; 6,174,666; 6,291,664; 6,414,132; and 6,794,498, accordingly.

Polynucleotides can be, e.g., in the form of RNA or in the form of DNA. DNA includes cDNA, genomic DNA, and synthetic DNA. DNA can be double-stranded or single-stranded. If single stranded, DNA can be the coding strand or non-coding (anti-sense) strand. In some aspects of the present disclosure, the polynucleotide is a cDNA or a DNA lacking one or more introns. In some aspects of the present disclosure, a polynucleotide is a non-naturally occurring polynucleotide. In some aspects of the present disclosure, a polynucleotide is recombinantly produced. In some aspects of the present disclosure, the polynucleotides are isolated. In some aspects of the present disclosure, the polynucleotides are substantially pure. In some aspects of the present disclosure, a polynucleotide is purified from natural components.

In some aspects, vectors (e.g., expression vectors) comprise nucleotide sequences encoding anti-EL antibodies and antigen-binding fragments thereof or a domain thereof for recombinant expression in host cells, preferably in mammalian cells. In some aspects, cells, e.g. host cells, comprise such vectors for recombinantly expressing anti-EL antibodies or antigen-binding fragments thereof described herein (e.g., human or humanized antibodies or antigen-binding fragments thereof). Thus, a method for producing an antibody or antigen-binding fragment thereof described herein can comprise expressing such antibody or antigen-binding fragment thereof in a host cell.

An expression vector can be transferred to a cell (e.g., host cell) by conventional techniques and the resulting cells can then be cultured by conventional techniques to produce an antibody or antigen-binding fragment thereof described herein (e.g., an antibody or antigen-binding fragment thereof comprising the six CDRs, the VH, the VL, the VH and the VL, the heavy chain, the light chain, or the heavy and the light chain of MEDI5884) or a domain thereof (e.g., the VH, the VL, the VH and the VL, the heavy chain, or the light chain of MEDI5884).

In some aspects of the present disclosure, anti-EL antibodies or antigen-binding fragments thereof (e.g., an antibody or antigen-binding fragment thereof comprising the CDRs of MEDI5884) are administered according to the methods provided herein are produced in a host cell. In some aspects of the present disclosure, the host cell is a CHO cell.

In some aspects of the present disclosure, an antibody or antigen-binding fragment thereof administered according to the methods provided herein is isolated or purified. Generally, an isolated antibody or antigen-binding fragment thereof is one that is substantially free of other antibodies or antigen-binding fragments thereof with different antigenic specificities than the isolated antibody or antigen-binding fragment thereof. For example, in some aspects of the present disclosure, a preparation of an antibody or antigen-binding fragment thereof described herein is substantially free of cellular material and/or chemical precursors.

The following examples are offered by way of illustration and not by way of limitation.

### 6. EXAMPLES

The examples in this Section (i.e., Section 6) are offered by way of illustration, and not by way of limitation.

### 6.1 Example 1: Nonclinical Pharmacology of MEDI5884

Nonclinical in vivo pharmacology studies showed that administration of a single subcutaneous (SC) dose of MEDI5884 (0.5, 6, or 30 mg/kg) in normal male cynomolgus monkeys increased plasma HDL-C in a dose-dependent manner. The increase of HDL-C from baseline to maximum effect for the 0.5, 6, and 30 mg/kg doses was 63±14 to 96±29 mg/dL, 60±3.8 to 111±5.6 mg/dL and 54±7.4 to 122±17 mg/dL, respectively. Smaller increases in total cholesterol (TC), low-density lipoprotein cholesterol (LDL-C), and non-HDL-C (TC minus HDL-C) were also observed.

Apolipoprotein A1 (ApoA1), the major lipoprotein constituent of HDL, was also increased by 75±5.5% at the 30 mg/kg dose. A dose-dependent increase in serum phospholipids was observed, indicating that MEDI5884 inhibited hydrolysis of phospholipids contained in HDL particles.

Increases in ATP-binding cassette transporter A1 (ABCA1), cholesterol efflux capacity (CEC), global cholesterol efflux, and the total number of HDL particles were observed at the highest dose (30 mg/kg). Increases in the mean number of total and large low-density lipoprotein (LDL) particles were also observed. Cholesterol efflux was measured using HDL obtained from animals in the 0.5 and 30 mg/kg MEDI5884 treatment groups at 0, 0.5, 1, 2, 3, 7, and 14 days post-dose. ABCA1 efflux transiently decreased below baseline (Days 0.5 and 1) but exceeded baseline levels by 6%, 26%, and 114% on Days 3, 7, and 14, respectively, following treatment with 30 mg/kg MEDI5884. Similarly, global efflux transiently decreased below baseline on Day 0.5 (−6%) and increased to 6%, 5%, 41%, and 53% on Days 2, 3, 7, and 14, respectively.

HDL particles were characterized using NMR spectroscopy in samples obtained from cynomolgus monkeys treated with MEDI5884. Treatment with 30 mg/kg MEDI5884 increased the total number of HDL particles maximally by 49±6.3% and increased the number of both small and large particles by up to 182±85% and 104±10%, respectively. Medium HDL particles maximally decreased by 48±14% on Day 2 and returned to baseline by Day 49 suggesting speciation or interconversion of HDL particles as a new equilibrium is established. HDL size increased from baseline to plateau levels for the 0.5, 6, and 30 mg/kg doses from 10.0±0.033 to 10.9±0.17 nm, 10±0.23 to 10.7±0.12 nm, and 9.9±0.033 to 10.7±0.13 nm, respectively.

These data support the use of MEDI5884 in human patients, e.g., for prevention of secondary cardiovascular events.

### 6.2 Example 2: Nonclinical Pharmacokinetics and Safety of MEDI5884

MEDI5884 was administered to cynomolgus monkeys in a single dose non-Good Laboratory Practice (GLP) PK/pharmacodynamic (PD) study and 2 repeat-dose GLP toxicology studies. SC administration of MEDI5884 at single doses up to 30 mg/kg was well tolerated. There were no unscheduled deaths, adverse clinical observations, injection site reactions, or adverse effects on body weight. After repeated SC dosing (1 dose every 2 weeks) with 10, 30, or 100 mg/kg/dose of MEDI5884 for 1 month or for 6 months, all animals survived to their scheduled sacrifice. No MEDI5884-related changes in clinical observations, ophthalmic evaluations, body weight, behavior, neurophysiology, respiratory rate, injection site irritation scoring, heart rate, electrocardiograms (ECG), or blood pressure were observed. Treatment-related findings were limited to pharmacologically-mediated, minimal to moderate increases in TC, HDL-C, LDL-C, and phospholipids at all dose levels. Microscopically, the presence of minimal to moderate perivascular lymphocytic/mixed leukocytic infiltrate was observed at the SC injection sites of a few MEDI5884-treated animals; treatment-related histopathological findings were not observed for any other tissue. Based on these results the no observed adverse effect level (NOAEL) was determined to be 100 mg/kg/dose.

### 6.3 Example 3: Phase 1 Clinical Evaluation of MEDI5884

MEDI5884 was assessed in a Phase 1, first-time-in-human, blinded, placebo-controlled, single dose escalation (SDE) study to evaluate the safety, PK, and PD of MEDI5884 administered subcutaneously (SC) in healthy subjects not receiving statin therapy. Subjects were randomized in a 3:1 ratio to receive 30, 100, 300, or 600 mg of MEDI5884 or placebo; initial cohorts were 6 MEDI5884 and 2 placebo subjects per dose level. These cohorts were then replicated using subjects of Japanese ancestry to provide data to support the conduct of clinical studies in Japan. A total of 64 subjects were enrolled at a single site in the United States (US). Follow-up duration varied by cohort from 28 to 90 days post-dose.

Subjects received a single injection of MEDI5884 (or placebo) for the 30 and 100 mg doses, 3 SC injections (100 mg each) for the 300 mg dose, or 6 SC injections (100 mg each) for the 600 mg dose.

### Subjects

The median (SD) age of enrolled subjects was 35.9 (9) years; 93.9% were male, 53.1% were Asian, 28.1% were White, 14.1% were Black or African-American, and 4.7% reported multiple ethnicities; 90.6% were not of Hispanic ethnicity.

### Pharmacokinetics

Following single SC administration of MEDI5884 at doses of 30, 100, 300, and 600 mg, MEDI5884 exhibited nonlinear PK, likely due to target-mediated drug disposition, with greater than dose proportional Cmax and AUC observed. The PK parameters are summarized in Table 7.

TABLE 7
Summary Statistics (Mean ± SD) of Noncompartmental Analysis PK Parameters
Following MEDI5884 Administration

| | | Tₘₐₓ^{a} | Cmax | AUC₀₋ₗₐₛₜ | CL/F |
|---|---|---|---|---|---|
| Dose | Cohort | (day) | (μg/mL) | (μg · day/mL) | (L/day) |
| 30 mg | Western | 2 | 0.612 ± 0.698 | 2.59 ± 3.38 | NC |
| | (n = 6) | | | | |
| | Japanese-American | 2-6 | 1.41 ± 1.26 | 9.36 ± 8.99 | NC |
| | (n = 6) | | | | |
| 100 mg | Western | 2-6 | 7.79 ± 1.85 | 102 ± 45.9 | 0.793 ± 0.282 |
| | (n = 6) | | | | (n = 4) |
| | Japanese-American | 2-6 | 6.93 ± 3.48 | 78.0 ± 82.1 | 0.519 ± 0.149 |
| | (n = 6) | | | | (n = 2) |
| 300 mg | Western | 6 | 24.2 ± 5.48 | 379 ± 101 | 0.744 ± 0.149 |
| | (n = 6) | | | | |
| | Japanese-American | 6-12 | 25.7 ± 11.7 | 682 ± 312 | 0.491 ± 0.197 |
| | (n = 6) | | | | |
| 600 mg | Western | 6 | 68.3 ± 19.6 | 1710 ± 779 | 0.378 ± 0.164 |
| | (n = 6) | | | | (n = 5) |
| | Japanese-American | 6 | 78.4 ± 20.7 | 2330 ± 584 | 0.254 ± 0.0441 |
| | (n = 6) | | | | |

AUC₀₋ₗₐₛₜ = area under the concentration-time curve from time zero to time of last quantifiable concentration;
CL/F = systemic clearance;
Cₘₐₓ = maximum observed concentration;
n = number of subjects;
NC = not calculated due to insufficient data;
PK = pharmacokinetics;
SD = standard deviation;
Tₘₐₓ = time to maximum observed concentration
^{a}Range was reported for Tₘₐₓ except for cases where Tₘₐₓ was the same for all subjects.

The observed mean PK profiles between the Japanese-American population and the general US population (Western cohort) generally overlapped. Higher exposure was observed in the Japanese-American subjects at the 300 mg dose and was likely due to the difference in body weight between the Japanese-American and the general US populations. Modest effect of body weight on PK profiles in both Western and Japanese-American subjects was observed. The CL/F for the 600 mg dose was 0.378 L/day and 0.254 L/day in the general US (Western cohort) and Japanese-American populations. The data suggested no substantial ethnic differences in MEDI5884 PK.

### Safety and Immunogenicity

Safety data were evaluated for 64 subjects, including 48 who received MEDI5884 (4 dose cohorts [30, 100, 300, or 600 mg] of 12 subjects each; 24 were of Japanese ancestry) and 16 who received placebo (4 dose cohorts of 4 subjects each; 8 were of Japanese ancestry). There were no related treatment-emergent adverse events (TEAEs), treatment-emergent serious adverse events (TESAEs), or deaths that led to withdrawal from the study. TEAEs occurred in similar proportions of subjects who received MEDI5884 ( 16/48, 33.3%) or placebo ( 5/16, 31.3%).

No subject in the general US population had a positive ADA result; however, a positive anti-drug antibody (ADA) response was detected in 6 of 32 Japanese-American subjects, including 1 placebo recipient. No AEs were reported to be associated with the ADAs. Lower exposure was observed in some ADA-positive Japanese-American subjects compared to other Japanese-American subjects with no ADAs; the exposure in these ADA-positive subjects is within the exposure range of subjects at the same dose in the pooled population, and there was no observed effect on PD or safety. In sum, ADAs were uncommon, were not associated with adverse events (AEs) or effect on PD, and had no clinically relevant effect on PK.

### Pharmacodynamics

The overall baseline lipid levels were similar between the placebo subjects and MEDI5884-treated subjects.

In these healthy subjects, none of whom were receiving statin therapy, substantial increases in HDL-C were observed after MEDI5884 administration. The mean (SD) percent change from baseline in HDL-C at Day 28 was 4.1% (17.4), 42.0% (26.9), 39.7% (22.5), and 49.8% (17.3) in subjects who received MEDI5884 at 30, 100, 300, or 600 mg, respectively, versus 15.9% (16.8) for placebo.

Increases in apoA1 were also observed. Small increases in LDL-C and apoB were observed in the pooled population; these increases did not appear to be dose-related and occurred primarily late after dosing. No clear effect on triglyceride levels was observed.

### 6.4 Example 4: Phase 2a Clinical Evaluation of MEDI5884

MEDI5884 has also been assessed in a Phase 2a, randomized, double-blind, placebo-controlled, parallel-designed study to evaluate the safety, PK, PD, and immunogenicity of MEDI5884 in subjects with stable coronary heart disease (CHD) receiving concomitant high-intensity statin therapy and who had triglyceride levels ≤500 mg/dL and LDL-C≤100 mg/dL.

A total of 132 subjects received 3 monthly SC doses of placebo or MEDI5884 at doses of 50, 100, 200, 350, and 500 mg.

### Subjects

This study enrolled predominantly male (87.0%) subjects of white race (90.8%), and the median age at enrolment was 67 years of age. The overall baseline characteristics were similar between the placebo subjects and MEDI5884-treated subjects. Subjects who received 3 total doses of investigational product were as follows: 22/23 (95.7%) in the placebo group and 18/20 (90%), 22/24 (91.7%), 20/22 (90.9%), 20/21 (95.2%), and 21/22 (95.5%) in the MEDI5584 50, 100, 200, 350, and 500 mg groups, respectively. One of the subjects in the MEDI5884 50 mg group was included as having completed treatment despite having received only 2 doses due to missing the Dose 2 visit.

### Pharmacokinetics

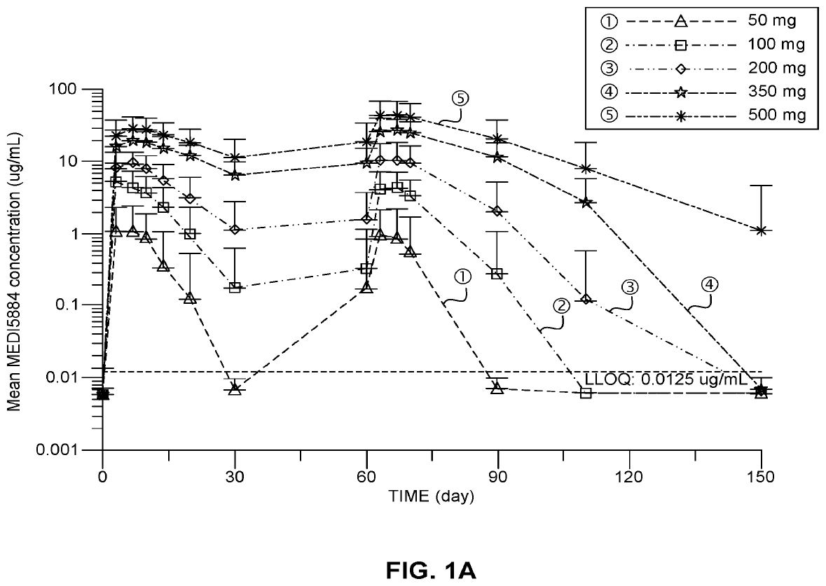

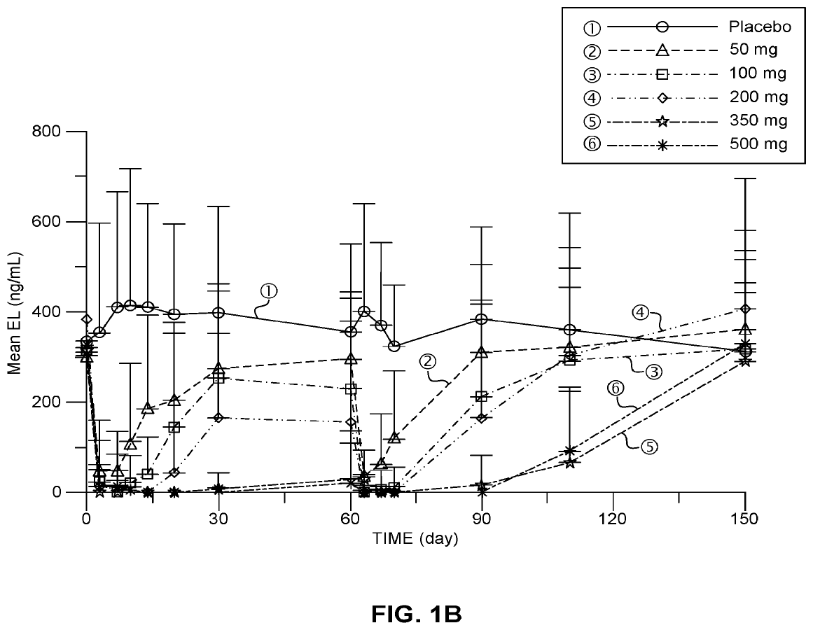

Interim PK analysis was conducted based on data up to Day 111. The mean MEDI5884 concentration-time profiles after three monthly SC doses of MEDI5884 are presented by dose cohort in FIG. 1A. PK parameters based on noncompartmental analysis are summarized in Table 8.

TABLE 8
Noncompartmental Analysis PK Parameters Following Last Dose (Day 60) of MEDI5884

| | | | Accumulation | AUC_{30 d} | Accumulation | | |
|---|---|---|---|---|---|---|---|
| | Tₘₐₓ | Cₘₐₓ | Ratio of Cₘₐₓ | (μg day/ | Ratio of AUC | CL/F | t_{l/2} |
| Dose | (day) | (μg/mL) | (Dose 1/Dose 3) | mL) | (Dose 1/Dose 3) | (L/day) | (days) |
| 50 mg | 64.4 ± 2.39 | 1.01 ± 1.34 | 1.10 ± 1.42 | 10.7 ± 16.4 | 1.61 ± 2.33 | NC | NC |
| (n = 18) | | | (n = 15) | | (n = 15) | | |
| 100 mg | 66.0 ± 2.34 | 4.63 ± 2.95 | 2.22 ± 6.27 | 67.9 ± 48.1 | 7.30 ± 28.2 | 0.639 | 7.56 |
| (n = 22) | | | (n = 21) | | (n = 21) | (n = 2) | (n = 2) |
| 200 mg | 66.5 ± 2.76 | 11.9 ± 7.77 | 1.33 ± 0.653 | 188 ± 148 | 1.46 ± 0.735 | 0.642 ± 0.249 | 8.09 ± 2.80 |
| (n = 20) | | | | | | (n = 7) | (n = 7) |
| 350 mg | 66.2 ± 3.01 | 28.5 ± 13.0 | 1.51 ± 0.583 | 572 ± 283 | 1.58 ± 0.643 | 0.458 ± 0.180 | 11.4 ± 4.95 |
| (n = 20) | | | | | | (n = 14 | (n = 14) |
| 500 mg | 66.6 ± 2.95 | 46.3 ± 24.6 | 1.56 ± 0.349 | 980 ± 609 | 1.61 ± 0.367 | 0.389 ± 0.209 | 12.9 ± 5.80 |
| (n = 20) | | | | | | (n = 12) | (n = 12) |

AUC_{30 d} = area under the concentration-time curve at 30 days; CL/F = apparent systemic clearance; Cₘₐₓ = maximum observed concentration; n = number of subjects; NC = not calculated due to insufficient data; t_{1/2} = half-life; PK = pharmacokinetic; Tₘₐₓ = time to maximum observed concentration.

MEDI5884 exhibited nonlinear PK, likely due to target-mediated drug disposition; however, PK in the linear range was observed at MEDI5884 doses of 350 and 500 mg after 30 days post-dose. The Cmax and AUC were generally greater than dose proportional. Large inter-subject variability and minor drug accumulation were observed. The mean estimated CL/F at 500 mg was 0.389 L/day.

### Safety and Immunogenicity

There were no deaths or related TESAEs, and AEs were generally balanced between the MEDI5884-treated ( 59/109 [54.1%]) and placebo groups ( 17/23 [73.9%]), were not dependent on MEDI5884 dose, and were representative of events expected to occur in the enrolled population. Self-reported injection site reactions occurred in 15/109 (14%) MEDI5884-treated subjects and 3/23 (13%) placebo-treated subjects. Investigator-reported injection site reactions occurred in 9/109 (8%) MEDI5884-treated subjects and 3/23 (13%) placebo-treated subjects; these reactions were mild to moderate in severity. Eight subjects were discontinued from dosing (1 placebo and 1 MEDI5884 recipient withdrew consent for dosing, and 6 others were withdrawn for laboratory observations as mandated by the protocol [some recorded as AEs; 4 for elevated apoB, and 2 for elevated triglycerides]).

ADAs were uncommon, low titer, and were not associated with AEs and had no effect on PK. ADAs were also similar in placebo and MEDI5884 recipients. Thus, the observed ADAs may represent false positives.

### Pharmacodynamic Effects

The overall baseline lipid levels were similar between the placebo subjects and MEDI5884-treated subjects.

Target engagement of MEDI5884, which is suppression of EL, was shown to be dose-dependent (FIG. 1B). In particular, the amount of hEL bound by MEDI5884 in human plasma was measured using the Meso Scale Diagnostics (MSD) based immunoassay platform. Briefly, wells on a 96-well plate were coated with MEDI5884 and incubated overnight at 4° C. The next day, wells were washed with PBS containing 0.05% Tween20 wash buffer and blocked with I-Block Buffer (Applied Biosystems) for one hour at room temperature. Plates were washed. Then recombinant EL protein standard (Origene Technologies) and human plasma samples were added to corresponding wells and incubated for one hour at room temperature. After washing, a biotinylated EL detection antibody (Origene Technologies) was added to corresponding wells and incubated for one hour at room temperature. Plates were washed, then a streptavidin, sulfo-TAG antibody (MSD) was added to corresponding wells and incubated for one hour at room temperature. After washing, wells were incubated with Read buffer (MSD). Plates were read using the MESO Sector S 600 plate reader, and data was analyzed using the MSD Discovery Workbench software analysis program.

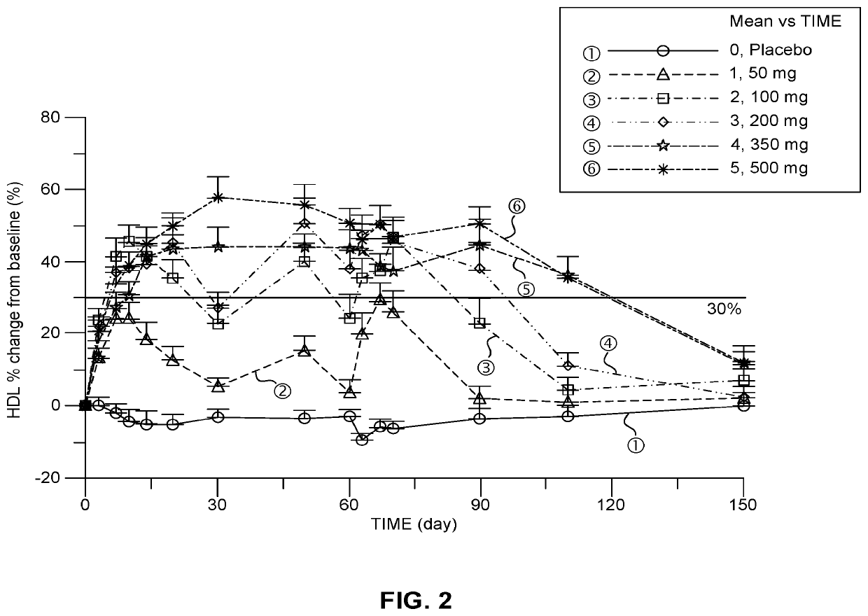

In these patients, dose-dependent increases from baseline in HDL-C were observed in MEDI5884-treated groups. The mean (SD) percent change from baseline in HDL-C at Day 91 (using the Last Observation Carried Forward (LOCF) imputation method) was 2.88% (14.86), 21.82% (30.66), 34.41% (34.89), 43.29% (31.09), and 48.31% (25.63) in subjects who received MEDI5884 at 50, 100, 200, 350, or 500 mg, respectively, versus −3.01% (13.60) for placebo. FIG. 2 and Tables 9A and 9B show the observed (non-LOCF) percent change from baseline in HDL-C over a 90 day period, and the observed (non-LOCF) change from baseline in HDL-C at Day 91.

TABLE 9A
HDL-C, mean % change from baseline

| | | MEDI5884 | MEDI5884 | MEDI5884 | MEDI5884 | MEDI5884 |
|---|---|---|---|---|---|---|
| | Placebo | 50 mg | 100 mg | 200 mg | 350 mg | 500 mg |
| Visit | N = 23 | N = 20 | N = 24 | N = 22 | N = 21 | N = 22 |
| BL, | 42.1 | 41.3 | 43.2 | 42.9 | 44.6 | 42.5 |
| mg/dL* | | | | | | |
| Day 30*\hat{ } | −3.08 | 7.37 | 23.0 | 27.3 | 44.1 | 55.8 |
| | 0/23 (0%) | 1/19 (5.3%) | 6/24 (25%) | 11/22 (50%) | 15/21 (71%) | 18/21 (86%) |
| Day 60*\hat{ } | −4.69 | 4.58 | 28.3 | 37.7 | 39.7 | 49.6 |
| | 0/18 (0%) | 1/17 (5.9%) | 10/20 (50%) | 10/18 (56%) | 14/18 (78%) | 15/18 (83%) |
| Day 90\hat{ } | −3.62 | 1.98 | 21.8 | 35.6 | 43.3 | 48.3 |
| | 0/21 (0%) | 0/19 (0%) | 6/24 (25%) | 11/21 (52%) | 15/21 (71%) | 15/22 (68%) |
| Mean of | −3.09 | 4.32 | 23.7 | 33.0 | 43.4 | 51.2 |
| Day 30, | 0/23 (0%) | 0/19 (0%) | 6/24 (25%) | 12/22 (55%) | 14/21 (67%) | 19/22 (86%) |
| 60, 90\hat{ } | | | | | | |

BL, baseline; *Day of dosing; \hat{ }proportion and percentage of subjects achieving a ≥30% increase in HDL-C.

TABLE 9B
HDL-C, mean % change from baseline

| Total HDL-C | | MEDI5884 | | | | | |
|---|---|---|---|---|---|---|---|
| (mg/dL) | Placebo | 50 mg | 100 mg | 200 mg | 350 mg | 500 mg | Total |
| Day 91- | N = 23 | N = 20 | N = 24 | N = 22 | N = 21 | N = 22 | N = 109 |

Change from Baseline

| | | | | | | | |
|---|---|---|---|---|---|---|---|
| n | 21 | 19 | 24 | 21 | 21 | 22 | 107 |
| Mean | −1.6 | 0.6 | 10.0 | 15.2 | 18.7 | 20.1 | 13.1 |
| SD | 5.5 | 5.7 | 17.1 | 15.5 | 13.9 | 10.9 | 14.9 |
| Median | −3.0 | 1.0 | 4.5 | 11.0 | 14.0 | 19.0 | 10.0 |
| (Min, Max) | (−15, 10) | (−16, 8) | (−5, 61) | (−11, 42) | (3, 53) | (4, 44) | (−16, 61) |

Percent Change from Baseline

| | | | | | | | |
|---|---|---|---|---|---|---|---|
| n | 21 | 19 | 24 | 21 | 21 | 22 | 107 |
| Mean | −3.62 | 1.98 | 21.82 | 35.63 | 43.29 | 48.31 | 30.67 |
| SD | 13.41 | 14.69 | 30.66 | 35.27 | 31.09 | 25.63 | 32.47 |
| Median | −6.15 | 3.13 | 11.10 | 34.38 | 37.14 | 51.76 | 24.39 |
| (Min, Max) | (−28.8, 29.4) | (−41.0, 25.0) | (−9.4, 111.3) | (−13.8, 113.8) | (8.2, 120.5) | (−9.5, 103.0) | (−41.0, 120.5) |

Dose-dependent increases from baseline in HDL particle number and HDL particle size (Table 10) were also observed in MEDI5884-treated groups relative to the placebo group.

TABLE 10
Lipoprotein Particle Size and Number Results by NMR

| | | MEDI5884 | | | | | |
|---|---|---|---|---|---|---|---|
| | Placebo | 50 mg | 100 mg | 200 mg | 350 mg | 500 mg | Total |
| | N = 23 | N = 20 | N = 24 | N = 22 | N = 21 | N = 22 | N = 109 |
| Total HDL-P | | | | | | | |
| (umol/L) Day 91-LOCF | | | | | | | |
| Change from baseline | | | | | | | |
| n | 23 | 19 | 23 | 22 | 20 | 22 | 106 |
| Mean | 0.28 | 0.95 | 1.63 | 1.78 | 1.66 | 2.90 | 1.81 |
| SD | 2.32 | 2.26 | 2.17 | 3.14 | 2.78 | 3.46 | 2.83 |
| Median | 0.30 | 1.20 | 1.50 | 1.45 | 1.25 | 2.90 | 1.60 |
| (Min, Max) | (−3.3, 5.5) | (−3.9, 6.1) | (−1.6, 6.0) | (−3.9, 7.8) | (−3.1, 5.6) | (−6.1, 10.7) | (−6.1, 10.7) |
| Total HDL-P (umol/L) | | | | | | | |
| Day 91-LOCF Percent | | | | | | | |
| Change from baseline | | | | | | | |
| n | 23 | 19 | 23 | 22 | 20 | 22 | 106 |
| Mean | 1.59 | 5.24 | 9.31 | 9.28 | 8.39 | 14.55 | 9.49 |
| SD | 11.01 | 12.02 | 12.31 | 15.30 | 13.69 | 15.54 | 13.94 |
| Median | 1.50 | 6.38 | 7.21 | 6.96 | 5.80 | 14.44 | 8.24 |
| (Min, Max) | (−18, 9, 22.3) | (−20.3, 33.9) | (−7.0, 38.3) | (−12.6, 40.5) | (−12.9, 29.3) | (−22, 0, 47.3) | (−22.0, 47.3) |
| HDL Size (nm) | | | | | | | |
| Day 91-LOCF | | | | | | | |
| Change from baseline | | | | | | | |
| n | 23 | 19 | 23 | 22 | 20 | 22 | 106 |
| Mean | −0.08 | 0.09 | 0.13 | 0.36 | 0.47 | 0.46 | 0.30 |
| SD | 0.20 | 0.22 | 0.32 | 0.27 | 0.28 | 0.32 | 0.32 |
| Median | −0.10 | 0.10 | 0.10 | 0.40 | 0.40 | 0.40 | 0.30 |
| (Min, Max) | (−0.4, 0.2) | (−0.2, 0.5) | (−0.6, 1.1) | (−0.1, 0.8) | (0.1, 1.2) | (0.0, 1.2) | (−0.6, 1.2) |
| HDL Size (nm) | | | | | | | |
| Day 91-LOCF Percent | | | | | | | |
| Change from baseline | | | | | | | |
| n | 23 | 19 | 23 | 22 | 20 | 22 | 106 |
| Mean | −0.84 | 1.01 | 1.44 | 4.11 | 5.34 | 5.35 | 3.47 |
| SD | 2.22 | 2.51 | 3.53 | 3.07 | 3.12 | 3.55 | 3.65 |
| Median | −1.16 | 1.08 | 1.16 | 4.62 | 4.73 | 4.73 | 3.41 |
| (Min, Max) | (−4.4, 2.4) | (−2.4, 5.9) | (−6.5, 12.1) | (−1.2, 9.2) | (1.2, 13.6) | (0.0, 13.3) | (−6.5, 13.6) |

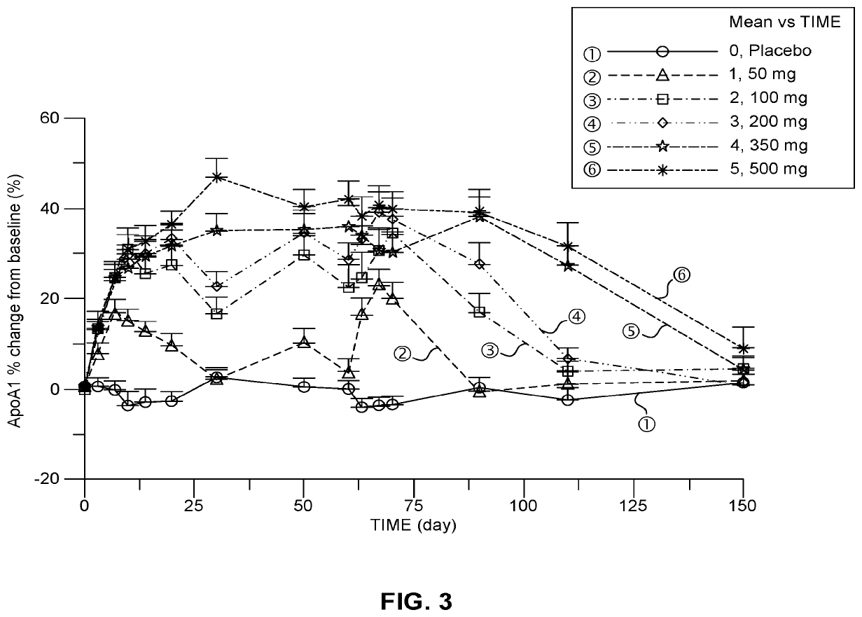

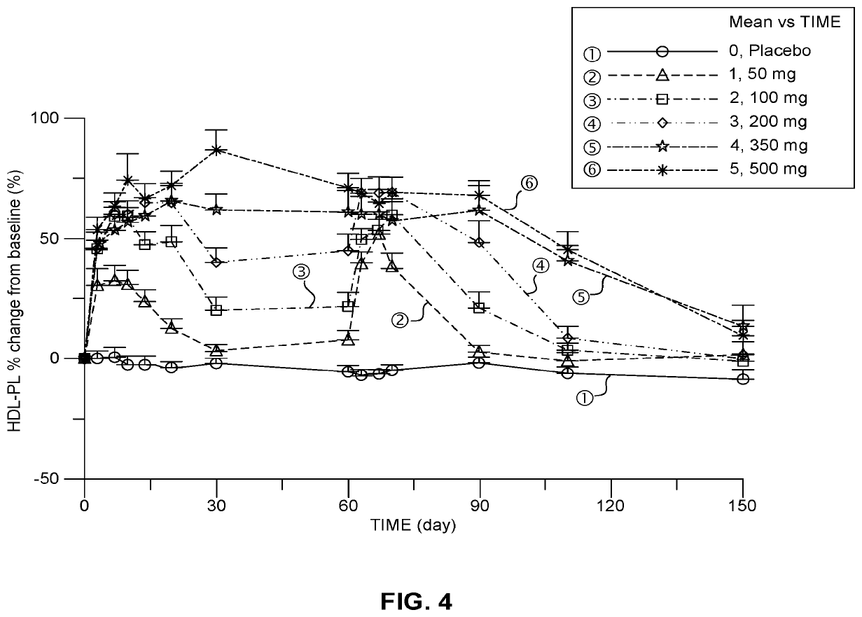

Dose-dependent increases from baseline in apoA1 (FIG. 3 and Tables 11A and 11B) and in high-density lipoprotein phospholipid (HDL-PL) (FIG. 4 and Table 12) were also observed in MEDI5884-treated groups relative to the placebo group. In particular, FIG. 3 and Tables 11A and 11B show the observed (non-LOCF) percent change from baseline in ApoA1 over a 90 day period, and the observed (non-LOCF) change from baseline in ApoA1 at Day 91. The mean (SD) percent change from baseline in ApoA1 at Day 91 (using the LOCF imputation method) was 1.32% (14.81), 15.88% (19.66), 24.82% (21.92), 36.26% (27.36), and 36.85% (18.03) in subjects who received MEDI5884 at 50, 100, 200, 350, or 500 mg, respectively, versus 1.42% (11.20) for placebo.

TABLE 11A
ApoA1, mean % change from baseline

| | | MEDI5884 | MEDI5884 | MEDI5884 | MEDI5884 | MEDI5884 |
|---|---|---|---|---|---|---|
| | Placebo | 50 mg | 100 mg | 200 mg | 350 mg | 500 mg |
| Visit | N = 23 | N = 20 | N = 24 | N = 22 | N = 21 | N = 22 |
| BL, g/L* | 1.257 | 1.263 | 1.262 | 1.313 | 1.309 | 1.303 |
| Day 30*\hat{ } | 2.93 | 3.54 | 16.8 | 22.7 | 35.2 | 45.1 |
| | 0/23 (0%) | 0/19 (0%) | 5/24 (21%) | 6/22 (27%) | 12/21 (57%) | 18/21 (86%) |
| Day 60*\hat{ } | −1.39 | 4.43 | 23.5 | 29.5 | 32.5 | 42.6 |
| | 0/18 (0%) | 0/17 (0%) | 7/20 (35%) | 10/18 (56%) | 9/18 (50%) | 14/18 (78%) |
| Day 90\hat{ } | 0.29 | −0.47 | 15.9 | 26.0 | 36.3 | 36.9 |
| | 0/21 (0%) | 0/19 (0%) | 6/24 (25%) | 8/21 (38%) | 12/21 (57%) | 14/22 (64%) |
| Mean of | 1.13 | 2.07 | 18.4 | 25.8 | 35.5 | 40.8 |
| Day 30, | 0/23 (0%) | 0/19 (0%) | 5/24 (21%) | 8/22 (36%) | 11/21 (52%) | 17/22 (77%) |
| 60, 90\hat{ } | | | | | | |

BL, baseline; *Day of dosing; \hat{ }proportion and percentage of subjects achieving a ≥30% change from baseline in ApoA1.

TABLE 11B
ApoA1, mean % change from baseline

| ApoA1 | | MEDI5884 | | | | | |
|---|---|---|---|---|---|---|---|
| (mg/dL) | Placebo | 50 mg | 100 mg | 200 mg | 350 mg | 500 mg | Total |
| Day 91 | N = 23 | N = 20 | N = 24 | N = 22 | N = 21 | N = 22 | N = 109 |

Change from Baseline

| | | | | | | | |
|---|---|---|---|---|---|---|---|
| n | 21 | 19 | 24 | 21 | 21 | 22 | 107 |
| Mean | 0.4 | −1.2 | 19.1 | 34.2 | 46.8 | 48.1 | 29.9 |
| SD | 13.0 | 16.3 | 25.1 | 28.5 | 34.3 | 24.9 | 31.7 |
| Median | 0 | −1.0 | 13.0 | 32.0 | 40.0 | 49.5 | 25.0 |
| (Min, Max) | (−18, 25) | (−44, 23) | (−15, 87) | (−17, 92) | (−7, 127) | (−9, 91) | (−44, 127) |

Percent Change from Baseline

| | | | | | | | |
|---|---|---|---|---|---|---|---|
| n | 21 | 19 | 24 | 21 | 21 | 22 | 107 |
| Mean | 0.29 | −0.47 | 15.88 | 26.00 | 36.26 | 36.85 | 23.27 |
| SD | 11.03 | 12.78 | 19.66 | 21.73 | 27.36 | 18.03 | 24.35 |
| Median | 0.00 | −0.93 | 10.13 | 28.91 | 33.01 | 38.93 | 19.53 |
| (Min, Max) | (−15.3, 21.9) | (−30.3, 19.4) | (−12.4, 62.1) | (−13.2, 67.3) | (−6.1, 98.4) | (−6.5, 62.2) | (−30.3, 98.4) |

TABLE 12
HDL Phospholipids (HDL-PL)

| | | MEDI5884 | | | | | |
|---|---|---|---|---|---|---|---|
| | Placebo | 50 mg | 100 mg | 200 mg | 350 mg | 500 mg | Total |
| | N = 23 | N = 20 | N = 24 | N = 22 | N = 21 | N = 22 | N = 109 |
| HDL-PL | | | | | | | |
| (mg/dL) Day 91- | | | | | | | |
| LOCF Change | | | | | | | |
| from baseline | | | | | | | |
| n | 23 | 19 | 24 | 22 | 20 | 21 | 106 |
| Mean | −0.6 | 3.1 | 20.5 | 41.4 | 57.5 | 56.7 | 35.8 |
| SD | 10.3 | 14.2 | 34.2 | 36.9 | 42.0 | 28.8 | 38.4 |
| Median | 0.0 | 4.0 | 8.0 | 46.0 | 51.0 | 60.0 | 30.0 |
| (Min, Max) | (−20, 22) | (−44, 24) | (−10, 130) | (−4, 112) | (−10, 170) | (−10, 118) | (−44, 170) |
| HDL-PL | | | | | | | |
| (mg/dL) Day 91- | | | | | | | |
| LOCF Percent | | | | | | | |
| Change from | | | | | | | |
| baseline | | | | | | | |
| n | 23 | 19 | 24 | 22 | 20 | 21 | 106 |
| Mean | −0.14 | 3.78 | 20.71 | 45.00 | 58.76 | 63.53 | 38.38 |
| SD | 11.56 | 13.74 | 29.19 | 40.95 | 43.15 | 31.02 | 39.72 |
| Median | 0.00 | 4.35 | 9.41 | 38.99 | 59.84 | 67.92 | 30.52 |
| (Min, Max) | (−21.3, 26.2) | (−40.0, 23.9) | (−9.4, 114.0) | (−5.7, 119.1) | (−10.9, 173.5) | (−12.2, 120.6) | (−40.0, 173.6) |

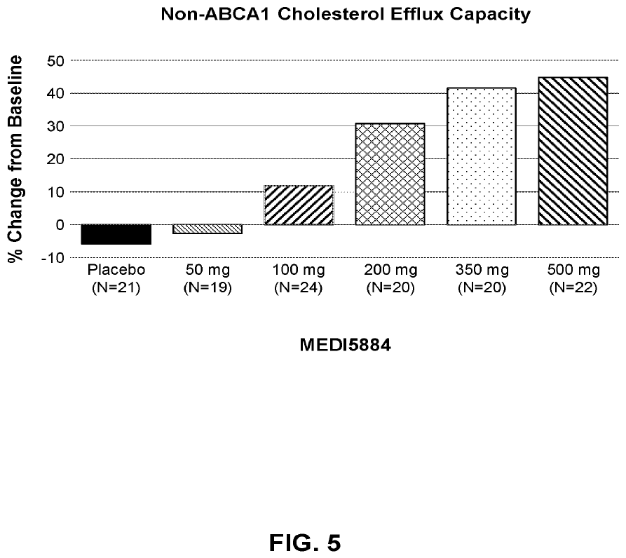

Dose-dependent increases from baseline in ABCA1-mediated efflux and global efflux were also observed in MEDI5884-treated groups relative to the placebo group. The effects of MEDI5884 on non-ABCA1 cholesterol efflux are shown in FIG. 5.

Modest increases in triglycerides, LDL-C, and apoB were also observed in the MEDI5884-treated groups. In lower dosage groups, there was no apparent dose-dependent relationship in the increases of triglycerides, LDL-C, and apoB in MEDI5884-treated groups. ApoB changes were only significant at the 500 mg dose. The observed ApoB levels at Day 91 across doses are shown in Table 13.

TABLE 13
ApoB levels

| | | MEDI5884 | MEDI5884 | MEDI5884 | MEDI5884 | MEDI5884 |
|---|---|---|---|---|---|---|
| Day of | Placebo | 50 mg | 100 mg | 200 mg | 350 mg | 500 mg |
| Visit | N = 23 | N = 20 | N = 24 | N = 22 | N = 21 | N = 22 |
| 91* | 4/21 (19%) | 4/19 (21%) | 4/24 (17%) | 4/21 (19%) | 7/21 (33%) | 9/22 (41%) |

*proportion and percentage of subjects with change from baseline ApoB levels ≥10 mg/dL.

Triglycerides increased to near or above 1000 mg/dL in 3 MEDI5884-treated subjects; these subjects had additional risk factors for hypertriglyceridemia. No dose-dependent effect of MEDI5884 on triglyceride levels was observed.

MEDI5884 at a dose of 200 mg resulted in a mean (median) change from baseline of 15.2 (11.0) mg/dL in HDL-C, 6.1 (5.0) mg/dL in LDL-C (direct), and 2 (−1.0) mg/dL in apoB at Day 91 compared to placebo (−1.6 [−3.0] mg/dL for HDL-C, −2.6 [−1.0] mg/dL for LDL-C, and −0.5 [0.0] mg/dL for apoB).

Key PK/PD model-estimated parameters are summarized in Table 14.

TABLE 14
PK/PD model estimated parameters

| | |
|---|---|
| Parameter (unit) | Model Estimate |

PK

| | |
|---|---|
| Vₘₐₓ (mg/day) | 4.8 |
| WT impact on Vₘₐₓ as a power function | 1.38 |
| ELBL impact on Vₘₐₓ as a power function | 0.419 |
| Kₘ (μg/mL) | 0.409 |
| CL (L/day) | 0.298 |
| V1 (L) | 4.44 |
| WT on V1 | 1.84 |
| CL_{d} (L/day) | 1.76 |
| V2 (L) | 5.41 |
| Kₐ (1/day) | 0.149 |

HDL-C

| | |
|---|---|
| kᵢₙ (mg/dL/day) | 11.3 |
| kₒᵤₜ (1/day) | 0.27 |
| IC₅₀ (μg/mL) | 0.337 |
| Iₘₐₓ | 0.362 |
| γ | 0.92 |

ApoA1

| | |
|---|---|
| kᵢₙ (mg/dL/day) | 25.6 |
| kₒᵤₜ (1/day) | 0.201 |
| IC₅₀ (μg/mL) | 0.381 |
| Iₘₐₓ | 0.307 |
| γ | 0.928 |

CL = clearance;
CL_{d} = intercompartmental clearance;
ELBL = baseline endothelial lipase levels;
γ = hill coefficient in the maximum effect (Eₘₐₓ) model;
HDL-C = high-density lipoprotein cholesterol;
Iₘₐₓ = maximal inhibition effect;
IC₅₀ = concentration to reach 50% of maximal effect (estimated simultaneous with Kₘ);
Kₐ = absorption rate constant;
Kᵢₙ = production rate;
Kₘ = concentration to reach 50% Vₘₐₓ;
Kₒᵤₜ = elimination rate;
PD = pharmacodynamic;
PK = pharmacokinetic;
V1 = central volume;
V2 = peripheral volume;
Vₘₐₓ = maximum contribution of dose-dependent nonlinear clearance;
WT = baseline body weight

### 6.5 Example 5: Phase 2B Clinical Evaluation of MEDI5884

An analysis of the data discussed above was performed to select a dose of 250 mg SC monthly MEDI5884 for further evaluation.

More specifically, a review of the data showed that administration of five doses of MEDI5884 showed a clear dose-dependent increase in exposure in the patients (FIG. 1A), which led to dose-dependent target engagement (i.e., inhibition of EL level) (FIG. 1B). The EL level of 200 mg dosing was not consistently inhibited for approximately 30 days, whereas that of 350 mg dosing was maintained full inhibition during the dosing interval of 30 days. Considering the monthly dosing interval, the optimal dose appeared to be between the studied dose 200 mg and 350 mg.

Biomarkers such as HDL-C, ApoA1, and HDL-PL increased with dose dependency after inhibiting EL activity (FIGS. 2-4). The time courses of the biomarkers indicated that the efficacious dose should be above 200 mg. Safety biomarkers in the pathway such as LDL-C, ApoB, and TG increased with less clear dose dependency compared to the efficacy biomarkers. Increase in ApoB and TG at 500 mg dose was identified as a concern, which indicates that a dose less than 500 mg dose should be chosen as an optimal dose.

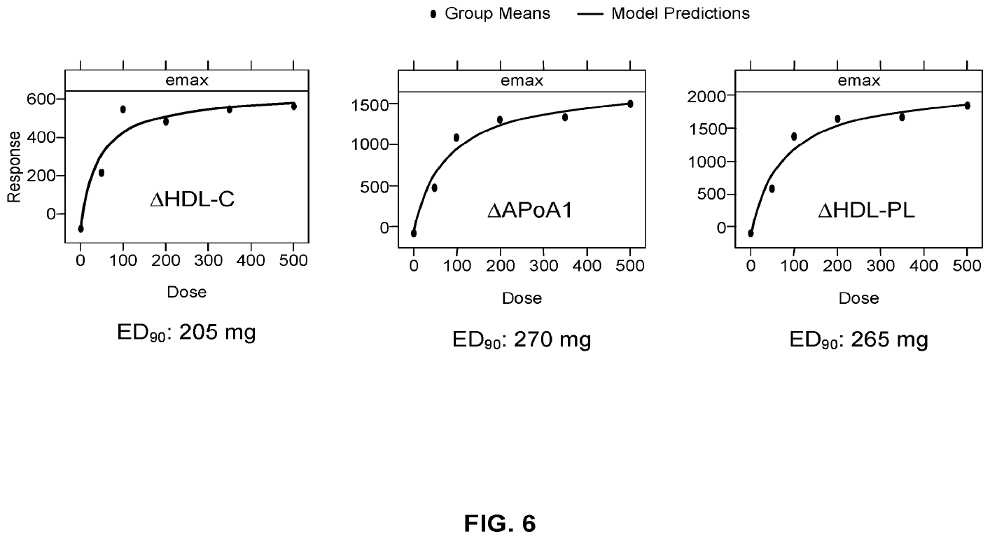

A Multiple Comparison Procedure Modeling (MCP Mod) method was applied to evaluate relationships of the Area-Under-the-Effect-Curve (AUEC) of HDL-C, ApoA1, HDL-PL, LDL, ApoB, and TG with respect to doses during the period from Day 60 to Day 90. The desirable biomarkers of HDL-C, ApoA1 and HDL-PL levels achieved maximum with increasing doses (FIGS. 2-4), which allows estimation of the dose that achieves 90% of the maximum biomarker level (ED₉₀). The estimated ED₉₀ was 205, 270 and 265 mg, respectively (FIG. 6). The undesirable biomarkers of LDL, ApoB and TG did not achieve plateaus, but showed the following trends: (1) the LDL level continued to increase in the range of 50 to 500 mg, (2) the ApoB level was dose-independent except at 500 mg, and (3) the TG level was constant regardless of the administered dose. Therefore, the outcome from the MCP Mod approach supports that 250 mg monthly dosing is likely to achieve a level of 90% of the maximum efficacy of desirable biomarkers without causing high levels of undesirable biomarkers.

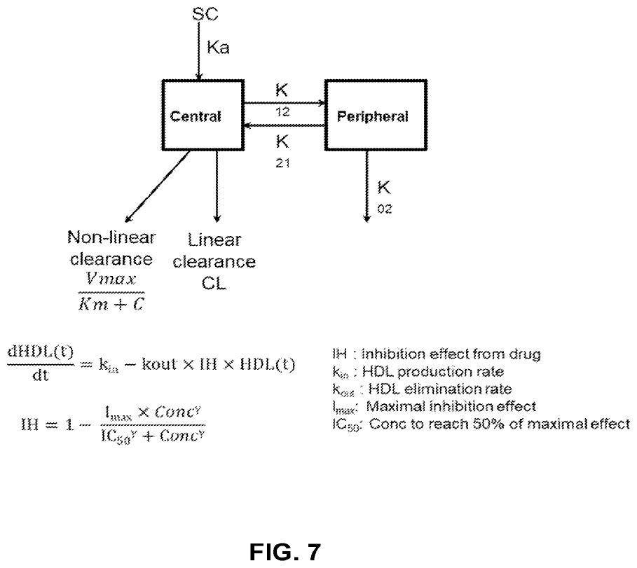

A mathematical model was developed to describe the pharmacokinetics (PK) of MEDI5884 and biomarker profiles of HDL and ApoA1, following administration of MEDI5884. The PK model part was with a 2-compartment PK model with parallel linear and nonlinear elimination pathways, whereas the PD model part for biomarker modeling followed the typical Indirect Response Model that has inhibition of the elimination pathway of each biomarker, which yielded increase in the biomarker level after dosing (FIG. 7). Although the model does not include HDL-P, by evaluating HDL-C and ApoA1, it indirectly addresses changes in HDL-P. An increase in HDL-C without a concomitant increase in ApoA1 translates into larger HDL particles but not particle number.

When the PK data from the wide dose range (50-500 mg) were analyzed simultaneously, MEDI5884 exhibited nonlinear PK due to target-mediated drug disposition, which is likely to be saturated at low doses. Therefore, it is reasonable to assume that the PK is linear at high doses for simulation of PK profiles at 250 mg, which is interpolation between the observed PK profiles after dosing 200 mg and 350 mg.

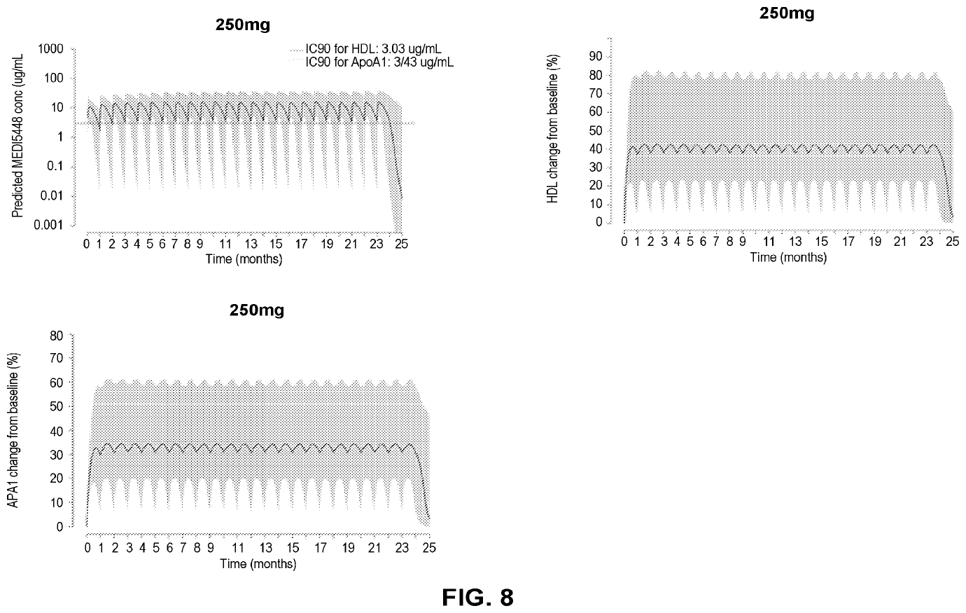

The observed PK/PD data from Phase 2a study of MEDI5884 in subjects with CHD who were receiving high-intensity statin therapy were well characterized by the model. Although large inter-subject variability was observed in PK, HDL, and ApoA1 after dosing, there was a clear relationship between MEDI5884 exposure and the corresponding increases in HDL-C and ApoA1. The model-estimated 50% inhibitory concentration (IC₅₀) values, which were used to calculate the IC₉₀ (i.e., 3.03 ug/mL and 3.43 ug/mL, for HDL-C and ApoA1, respectively). The predicted median trough concentration after 250 mg QM is approximately 3.46 ug/mL based on simulation below, which is close to the target exposure of IC₉₀ of HDL-C and ApoA1. The predicted time courses of MEDI5884, HDL, and ApoA1 after administration of a monthly dose of 250 mg of MEDI5884 are shown in FIG. 8.

Collectively, these data suggest that a monthly dose of 250 mg of MEDI5884 would exhibit the following: (i) linear PK and target engagement (EL suppression) for 30 days post dosing; (ii) a median trough level greater than the IC₉₀ for maximal HDL-C and ApoA1 increase based on PK/PD modeling; and (iii) minimal unwanted LDL-C and apoB increases based on a Multiple Comparison Procedure Modeling approach.

Accordingly, these data support an assessment of MEDI5884 in a Phase 2b, randomized, double-blind, placebo-controlled study in adults with prior myocardial infarction (MI) receiving high intensity statin therapy. In these studies, MEDI5884 (250 mg) or placebo is to be administered at a dose of 250 mg once a month for 24 months to demonstrate that MEDI5884 reduces the rate of cardiovascular death, MI, stroke, and coronary revascularization.

### 6.6 Example 6: Inhibition of EL and PCSK9 in Cynomolgus Monkeys

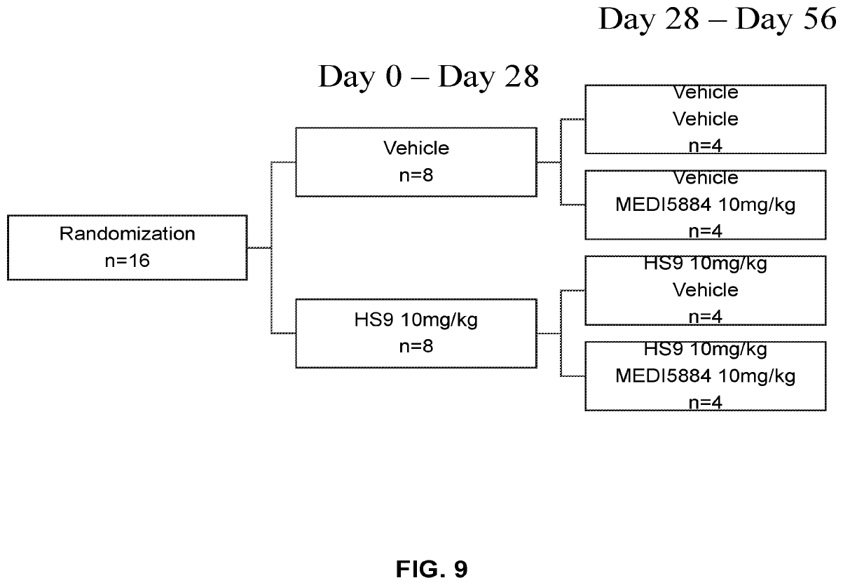

A combination pharmacology study to assess the impact of inhibition of proprotein convertase subtilisin/kexin type 9 (PCSK9) on lipoprotein metabolism following MEDI5884 treatment was conducted. The study protocol is depicted in FIG. 9. To better mimic a hypothetical patient population already on LDL-C lowering medication, healthy cynomolgus monkeys were first treated with weekly, subcutaneous injections of a PCSK9 neutralizing monoclonal antibody (mAb) (10 mg/kg; n=8) or vehicle for four weeks starting on Day 0 to establish a baseline of low LDL-C. The PCSK9 mAb used was HS9 (comprising the VH and VL sequences of SEQ ID NOs:14 and 15, respectively). The HS9 antibody (in the context of a GLP-1 fusion protein called MEDI4166) is disclosed in Chodorge et al., Sci. Rep. 8: 17545 (2018) and International PCT Publication No. WO2015127273, each of which is herein incorporated by reference in its entirety.

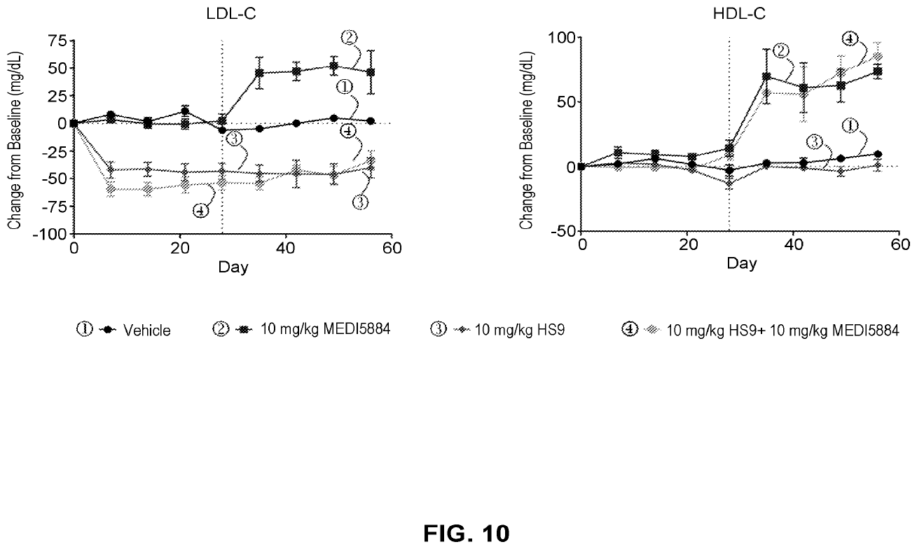

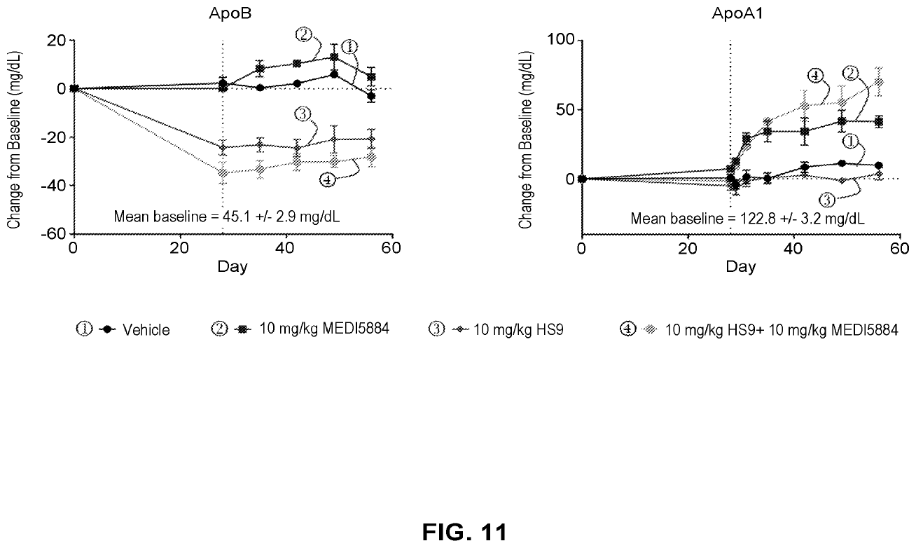

Four animals from each group were then administered subcutaneous doses of MEDI5884 (10 mg/kg; n=4) or vehicle (n=4) on Day 28 (vertical hashed line in FIGS. 10 and 11) and Day 42. LDL-C, HDL-C, ApoB, and ApoA1 were measured in plasma samples collected at the indicated timepoints. Global efflux and ABCA1 efflux were also assessed.

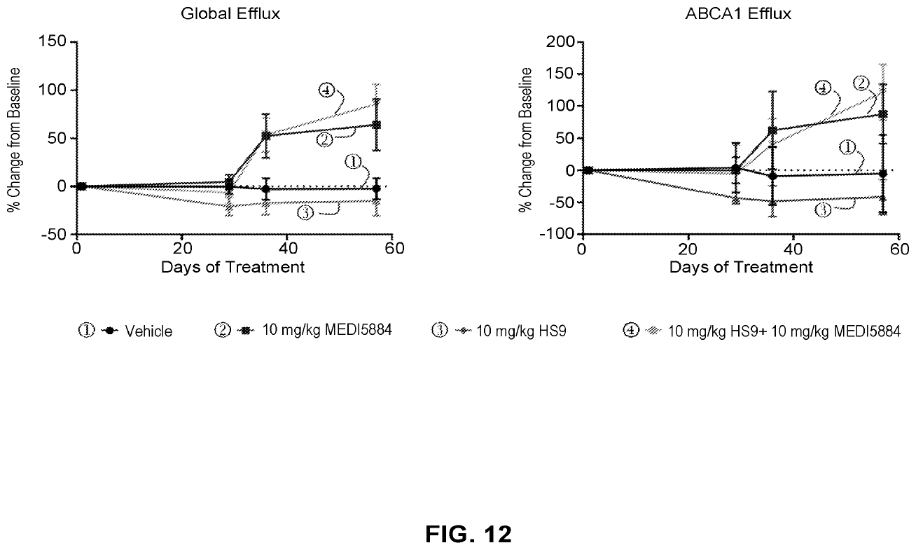

An increase in LDL-C was observed following EL neutralization. Compared to vehicle-treated animals, PCSK9 inhibition reduced LDL-C by 75% (FIG. 10, left graph) and maintained that level of reduction for the duration of the four-week lead-in period, with no appreciable effect on HDL-C (FIG. 10, right graph). In animals treated with vehicle during the lead-in period, MEDI5884 led to increases in both HDL-C and LDL-C. When added on top of the PCSK9 inhibitor during the second four-week period, MEDI5884 maintained the ability to raise HDL-C to a similar degree, but the magnitude of LDL-C increase was significantly blunted, indicating that the LDL particles continued to be taken up by the LDL receptor (FIG. 10). The patterns of LDL-C and HDL-C were matched by respective changes in ApoB and ApoA1 (FIG. 11). The effects on global efflux and ABCA1 efflux are shown in FIG. 12. In particular, the observed increases in efflux associated with administration of MEDI5884 (including administration of MEDI5884+HS9) generally reflect the observed increases in HDL-C.

These results provide evidence of cholesterol uptake by the LDL receptor and indicate that the increase in LDL-C observed with MEDI5884 treatment in monkeys can be mitigated by mechanisms that upregulate the LDL receptor. These results further indicate that MEDI5884 can be administered in combination with inhibitors of PCSK9. This combination therapy can take advantage of the combined action of the two complementary mechanisms both targeting different aspects of reverse cholesterol transport.

### 6.7 Example 7: Effect of Ascending Doses of MEDI5884 on Plasma Phosphatidylinositol (PI) Levels in Subjects with Stable Coronary Heart Disease

The effect of MEDI5884 on plasma phosphatidylinositol (PI) levels in patients with stable coronary heart disease were quantified. The quantification used a high throughput multiplex method with hydrophilic interaction chromatography (HILIC) separation coupled with multiple reaction monitoring (MRM) in negative mode. A total of 31 endogenous PI species were monitored.

### Reagents

Lipid standards were all purchased from Avanti Polar Lipids (Alabaster, Ala.). Three PI standards were used in this study: PI (12:0/13:0) used as internal standard (IS), PI (17:0/14:1), and PI (21:0/22:6) were used as surrogate analytes. High-performance liquid chromatography (HPLC)-grade water was purchased from Honeywell (Charlotte, N.C.). HPLC-grade isopropanol (IPA), acetonitrile (ACN), and ammonia were all purchased from Sigma-Aldrich (St. Louis, Mo.). Ammonium acetate and bovine serum albumin (BSA) were purchased from MilliporeSigma (Burlington, Mass.). PBS was purchased from Lonza BioWhittaker (Morristown, N.J.). Special glass-coated 96-well extraction plates and glass vials with PTFE (polytetrafluoroethylene) lining were obtained from Thermo-Fisher (Waltham, Mass.). Human plasma (pooled and individual) were purchased from BioIVT (Westbury, N.Y.).

### Extraction Procedure for Plasms Samples

Internal standard (IS) was spiked in isopropanol (IPA) at 60 nM (final concentration) to make IS-IPA solution. One pooled lot of human plasma was used as quality control (QC) and prepared at two levels: low QC (LQC) (8× dilution with 40 mg/mL bovine serum albumin (BSA) in PBS) and high QC (HQC) (undiluted plasma). Test samples were all diluted 8× with 40 mg/mL BSA. The 8× dilution of both HQC and test samples were prepared using automated liquid handling platform, Agilent Bravo Automated Liquid Handling System with Series III 96 LT Disposable Tip Head (Santa Clara, Calif.). 20 μL of LQC, HQC, or test sample was transferred to a separate extraction plate, following a predesignated plate map, and then was precipitated with 180 μL of IS-IPA utilizing automation mentioned above. Samples were shaken vigorously for about 10 minutes (900-1200 rpm) on IKA MTS 2/4 digital microtiter shaker (Wilmington, N.C.).

Samples were then centrifuged for 5 minutes at 2500 g. Automated liquid handling was used again to transfer 20 μL plasma extracted with IS-IPA to 140 μL of reconstitution solution (90.25%, v/v, acetonitrile (ACN), 9.75%, v/v, water, 10 mM ammonium acetate). Samples were then shaken for 5 minutes at 600 rpm.

### Hydrophilic Interaction Chromatography Separation

Chromatographic separation was performed on an Acquity ultra performance liquid chromatography (UPLC) BEH (Ethylene Bridged Hybrid) hydrophilic interaction chromatography (HILIC) column (130 Å, 1.7 μm, 2.1 mm×100 mm, Waters, Milford, Mass.) with a Nexera X2 UHPLC system (Shimadzu, Kyoto, Kyoto Prefecture, Japan). The mobile phases used were: mobile phase A (MPA) (5% water, 95% ACN, v/v) and mobile phase B (MPB) (50% water, 50% ACN, v/v) both with 10 mM ammonium acetate, pH 8.0-8.5. For each sample or QC, 10 μL of reconstituted IPA extract was injected onto the column. The separation was performed at 37° C. at a flow rate of 0.5 mL/min. The separation gradient was 5% to 13% MPA over 4 minutes, followed by a 2-minute wash period and a 4-minute equilibration period.

### Mass Spectrometry

Mass spectrometric detection was achieved using 6500+quadrupole ion trap (QTRAP) mass spectrometer (Sciex, Framingham, Mass.) operated in negative electrospray ionization (ESI) multiple-reaction monitoring (MRM) mode. The synthetic reference standard PI species listed above were used to tune the MS conditions and define the retention time. The highest signal intensity structurally characteristic MRM transition in negative ESI mode enabling identification of the two acyl chains was selected during the tuning of the synthetic reference standards (surrogate analytes and the IS). Structurally similar MRMs enabling identification of the two acyl chains of each endogenous PI species were then predicted using LipidView (Sciex, Redwood Shores, Calif.) and are listed in Table 15. After tuning the synthetic reference standards, the same source parameters were used for all PI species. The details for the acquisition method can be found in Table 16.

TABLE 15
List of MRMs

| | Precursor Ion | | |
|---|---|---|---|
| PI species | Detected [M − H]- | Analyte Type | Q1/Q3 |
| PI (12:0/13:0) | PI (12:0/13:0)-H | internal standard | 711.4/213.2 |
| PI (17:0/14:1) | PI (17:0/14:1)-H | surrogate | 793.5/269.2 |
| PI (21:0/22:6) | PI (21:0/22:6)-H | surrogate | 951.6/325.3 |
| PI (18:0/20:4) | PI (18:0/20:4)-H | endogenous | 885.6/283.3 |
| PI (18:0/18:2) | PI (18:0/18:2)-H | endogenous | 861.6/279.2 |
| PI (18:0/20:3) | PI (18:0/20:3)-H | endogenous | 887.6/283.3 |
| PI (18:0/18:1) | PI (18:0/18:1)-H | endogenous | 863.6/283.3 |
| PI (16:0/20:4) | PI (16:0/20:4)-H | endogenous | 857.5/255.2 |
| PI (16:0/18:2) | PI (16:0/18:2)-H | endogenous | 833.5/255.2 |
| PI (18:1/18:1) | PI (18:1/18:1)-H | endogenous | 861.6/281.2 |
| PI (18:1/16:0) | PI (18:1/16:0)-H | endogenous | 835.5/255.2 |
| PI (18:1/20:4) | PI (18:l/20:4)-H | endogenous | 883.5/281.2 |
| PI (18:1/18:2) | PI (18:1/18:2)-H | endogenous | 859.5/281.2 |
| PI (18:0/22:5) | PI (18:0/22:5)-H | endogenous | 911.6/283.3 |
| PI (18:0/20:2) | PI (18:0/20:2)-H | endogenous | 889.6/283.3 |
| PI (14:2/22:0) | PI (14:2/22:0)-H | endogenous | 861.6/223.2 |
| PI (16:0/20:3) | PI (16:0/20:3)-H | endogenous | 859.5/255.2 |
| PI (16:1/18:0) | PI (16:1/18:0)-H | endogenous | 835.5/283.3 |
| PI (18:0/22:4) | PI (18:0/22:4)-H | endogenous | 913.6/283.3 |
| PI (18:1/20:3) | PI (18:l/20:3)-H | endogenous | 885.6/281.2 |
| PI (16:0/16:1) | PI (16:0/16:1)-H | endogenous | 807.5/255.2 |
| PI (18:0/18:0) | PI (18:0/18:0)-H | endogenous | 865.6/283.3 |
| PI (16:0/18:0) | PI (16:0/18:0)-H | endogenous | 837.6/255.2 |
| PI (18:2/18:2) | PI (18:2/18:2)-H | endogenous | 857.5/279.2 |
| PI (16:0/16:0) | PI (16:0/16:0)-H | endogenous | 809.5/255.2 |
| PI (14:2/20:0) | PI (14:2/20:0)-H | endogenous | 833.5/223.2 |
| PI (14:2/22:2) | PI (14:2/22:2)-H | endogenous | 857.5/223.2 |
| PI (16:0/22:4) | PI (16:0/22:4)-H | endogenous | 885.6/255.2 |
| PI (16:0/20:2) | PI (16:0/20:2)-H | endogenous | 861.6/255.2 |
| PI (18:0/22:6) | PI (18:0/22:6)-H | endogenous | 909.6/283.3 |
| PI (14:2/22:1) | PI (14:2/22:1)-H | endogenous | 859.5/223.2 |
| PI (16:1/18:1) | PI (16:1/18:1)-H | endogenous | 833.5/281.2 |
| PI (18:0/18:3) | PI (18:0/18:3)-H | endogenous | 859.5/283.3 |
| PI (18:1/20:2) | PI (18:l/20:2)-H | endogenous | 887.6/281.2 |

TABLE 16
Operating conditions

| Parameter | Value |
|---|---|
| Source Temperature | 500.0° C. |
| Polarity: | Negative |
| Curtain Gas | 30 |
| Ion Source Gas 1 | 50 |
| Source Gas 2 | 60 |
| Ion Spray Voltage | −4500 V |
| Collision Energy | −62 V |
| Declustering Potential | −200 V |
| Collision Cell Exit Potential | −11.5 V |
| Entrance Potential | −10 V |

### Data Collection, Analysis and Reporting

After acquisition, the samples from each acquisition batch were analyzed in MultiQuant software following a defined quantification method (.qmethod). The details of the quantification method can be found in Table 17 for Components and Outlier Settings. Integration and Regression settings were optimized for each individual batch based on the peak of interest generated. However, identical Integration and Regression parameters were applied to all samples within the same batch. The internal standard peak area and endogenous PI species peak area were calculated for QC and unknown samples in the batch. The peak area ratios of the endogenous PI species were calculated based on the peak area integrations in MultiQuant. The MultiQuant quantification result file (.qsession) was then exported to an .txt file for further data analysis using Excel (Microsoft Office 2016) and Spotfire (TIBCO® Spotfire® Analyst 7.9.2 HF-011 Build version 7.9.2.0.12). In order to evaluate the linearity of instrument response and precision of measurement for each endogenous PI species, the following were assessed: ratio of HQC/LQC; % difference of HQC/LQC ratio from nominal; and % CV for HQC and LQC. The majority of species had % CV for HQC and LQC values of ≤30% and % difference of HQC/LQC ratio from nominal was within 70%-130%.

TABLE 17
MultiQuant procedures (components and outlier settings)

| Components | | | | | | | | Outlier Settings | |
|---|---|---|---|---|---|---|---|---|---|
| Acq. | Extraction | | Group | | | | | Accuracy For | Accuracy For |
| Index-1 | Type | Name | Name | IS | IS Name | Q1 Mass-1 | Q3 Mass-1 | QCs Used | Stds Used |
| 1 | MRM | PI(12:0/13:0)-H | PI(12:0/13:0) | TRUE | | 711.409 | 213.186 | FALSE | FALSE |
| 2 | MRM | PI(17:0/14:1)-H | PI(17:0/14:1) | FALSE | PI(12:0/13:0)-H | 793.487 | 269.2486 | FALSE | FALSE |
| 3 | MRM | PI(21:0/22:6)-H | PI(21:0/22:6) | FALSE | PI(12:0/13:0)-H | 951.597 | 325.3112 | FALSE | FALSE |
| 4 | MRM | PI(18:0/20:4)-H | PI(18:0/20:4) | FALSE | PI(12:0/13:0)-H | 885.55 | 283.2643 | FALSE | FALSE |
| 5 | MRM | PI(18:0/18:2)-H | PI(18:0/18:2) | FALSE | PI(12:0/13:0)-H | 861.55 | 279.233 | FALSE | FALSE |
| 6 | MRM | PI(18:0/20:3)-H | PI(18:0/20:3) | FALSE | PI(12:0/13:0)-H | 887.565 | 283.2643 | FALSE | FALSE |
| 7 | MRM | PI(18:0/18:1)-H | PI(18:0/18:1) | FALSE | PI(12:0/13:0)-H | 863.565 | 283.2643 | FALSE | FALSE |
| 8 | MRM | PI(16:0/20:4)-H | PI(16:0/20:4) | FALSE | PI(12:0/13:0)-H | 857.519 | 255.233 | FALSE | FALSE |
| 9 | MRM | PI(16:0/18:2)-H | PI(16:0/18:2) | FALSE | PI(12:0/13:0)-H | 833.519 | 255.233 | FALSE | FALSE |
| 10 | MRM | PI(18:1/18:1)-H | PI(18:1/18:1) | FALSE | PI(12:0/13:0)-H | 861.55 | 281.2486 | FALSE | FALSE |
| 11 | MRM | PI(18:1/16:0)-H | PI(18:1/16:0) | FALSE | PI(12:0/13:0)-H | 835.534 | 255.233 | FALSE | FALSE |
| 12 | MRM | PI(18:1/20:4)-H | Pl(18:1/20:4) | FALSE | PI(12:0/13:0)-H | 883.534 | 281.2486 | FALSE | FALSE |
| 13 | MRM | PI(18:1/18:2)-H | PI(18:1/18:2) | FALSE | PI(12:0/13:0)-H | 859.534 | 281.2486 | FALSE | FALSE |
| 14 | MRM | PI(18:0/22:5)-H | PI(18:0/22:5) | FALSE | PI(12:0/13:0)-H | 911.565 | 283.2643 | FALSE | FALSE |
| 15 | MRM | PI(18:0/20:2)-H | PI(18:0/20:2) | FALSE | PI(12:0/13:0)-H | 889.581 | 283.2643 | FALSE | FALSE |
| 16 | MRM | PI(14:2/22:0)-H | PI(14:2/22:0) | FALSE | PI(12:0/13:0)-H | 861.55 | 223.17 | FALSE | FALSE |
| 17 | MRM | PI(16:0/20:3)-H | PI(16:0/20:3) | FALSE | PI(12:0/13:0)-H | 859.534 | 255.233 | FALSE | FALSE |
| 18 | MRM | PI(16:1/18:0)-H | PI(16:1/18:0) | FALSE | PI(12:0/13:0)-H | 835.534 | 283.2643 | FALSE | FALSE |
| 19 | MRM | PI(18:0/22:4)-H | PI(18:0/22:4) | FALSE | PI(12:0/13:0)-H | 913.581 | 283.2643 | FALSE | FALSE |
| 20 | MRM | PI(18:1/20:3)-H | PI(18:1/20:3) | FALSE | PI(12:0/13:0)-H | 885.55 | 281.2486 | FALSE | FALSE |
| 21 | MRM | PI(16:0/16:1)-H | PI(16:0/16:1) | FALSE | PI(12:0/13:0)-H | 807.503 | 255.233 | FALSE | FALSE |
| 22 | MRM | PI(18:0/18:0)-H | PI(18:0/18:0) | FALSE | PI(12:0/13:0)-H | 865.581 | 283.2643 | FALSE | FALSE |
| 23 | MRM | PI(16:0/18:0)-H | PI(16:0/18:0) | FALSE | PI(12:0/13:0)-H | 837.55 | 255.233 | FALSE | FALSE |
| 24 | MRM | PI(18:2/18:2)-H | PI(18:2/18:2) | FALSE | PI(12:0/13:0)-H | 857.519 | 279.233 | FALSE | FALSE |
| 25 | MRM | PI(16:0/16:0)-H | PI(16:0/16:0) | FALSE | PI(12:0/13:0)-H | 809.519 | 255.233 | FALSE | FALSE |
| 26 | MRM | PI(14:2/20:0)-H | PI(14:2/20:0) | FALSE | PI(12:0/13:0)-H | 833.519 | 223.17 | FALSE | FALSE |
| 27 | MRM | PI(14:2/22:2)-H | PI(14:2/22:2) | FALSE | PI(12:0/13:0)-H | 857.519 | 223.17 | FALSE | FALSE |
| 28 | MRM | PI(16:0/22:4)-H | PI(16:0/22:4) | FALSE | PI(12:0/13:0)-H | 885.55 | 255.233 | FALSE | FALSE |
| 29 | MRM | PI(16:0/20:2)-H | PI(16:0/20:2) | FALSE | PI(12:0/13:0)-H | 861.55 | 255.233 | FALSE | FALSE |
| 30 | MRM | PI(18:0/22:6)-H | PI(18:0/22:6) | FALSE | PI(12:0/13:0)-H | 909.55 | 283.2643 | FALSE | FALSE |
| 31 | MRM | PI(14:2/22:1)-H | PI(14:2/22:1) | FALSE | PI(12:0/13:0)-H | 859.534 | 223.17 | FALSE | FALSE |
| 32 | MRM | PI(16:1/18:1)-H | PI(16:1/18:1) | FALSE | PI(12:0/13:0)-H | 833.519 | 281.2486 | FALSE | FALSE |
| 33 | MRM | PI(18:0/18:3)-H | PI(18:0/18:3) | FALSE | PI(12:0/13:0)-H | 859.534 | 283.2643 | FALSE | FALSE |
| 34 | MRM | PI(18:1/20:2)-H | PI(18:1/20:2) | FALSE | PI(12:0/13:0)-H | 887.565 | 281.2486 | FALSE | FALSE |

Period = 1; Experiment = 1

### Results

A total of 978 plasma samples from subjects with stable coronary heart disease were tested as described above. Samples were analyzed in a total of 15 assays. All 15 assays met acceptance criteria, for all PI species, except PI(16:0/16:0), which had consistently unacceptable variability and HQC/LQC ratio. No assay was considered invalid.

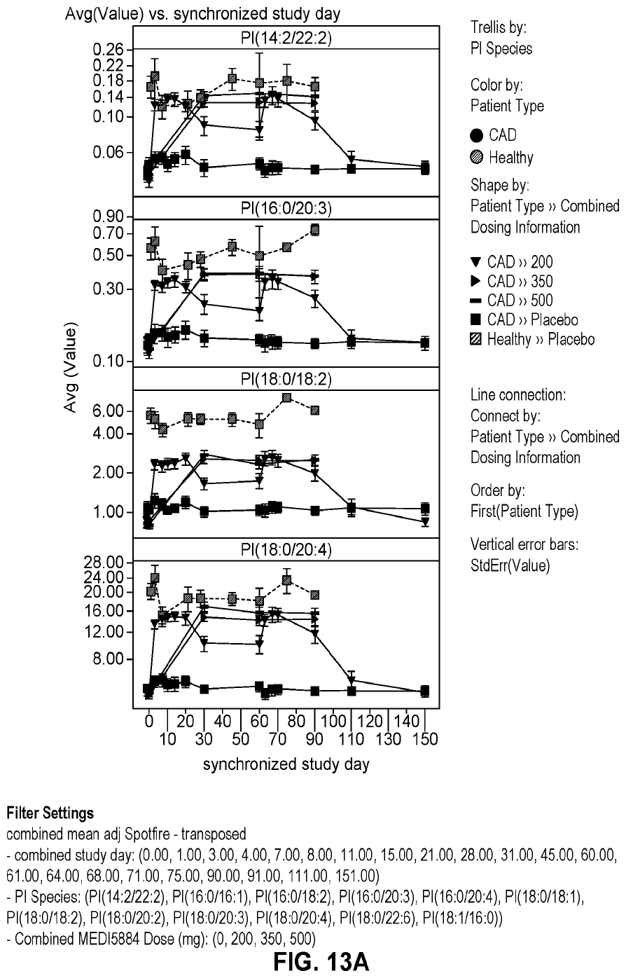

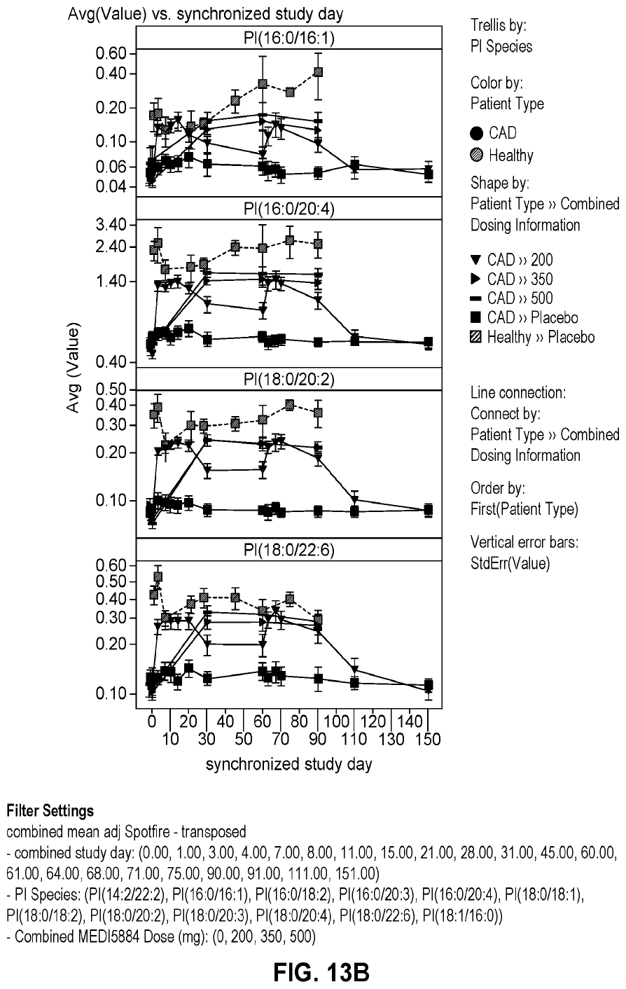

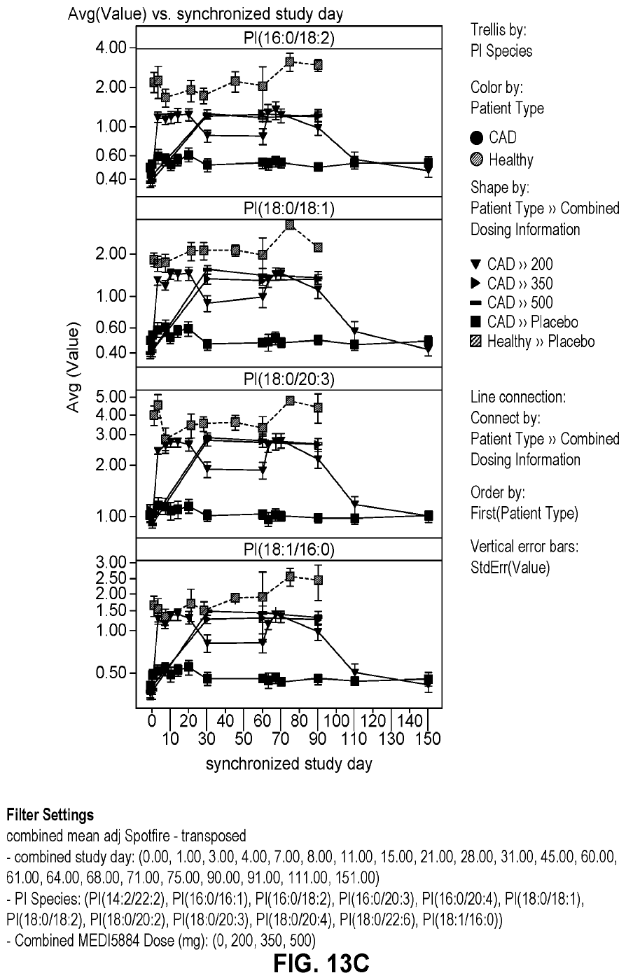

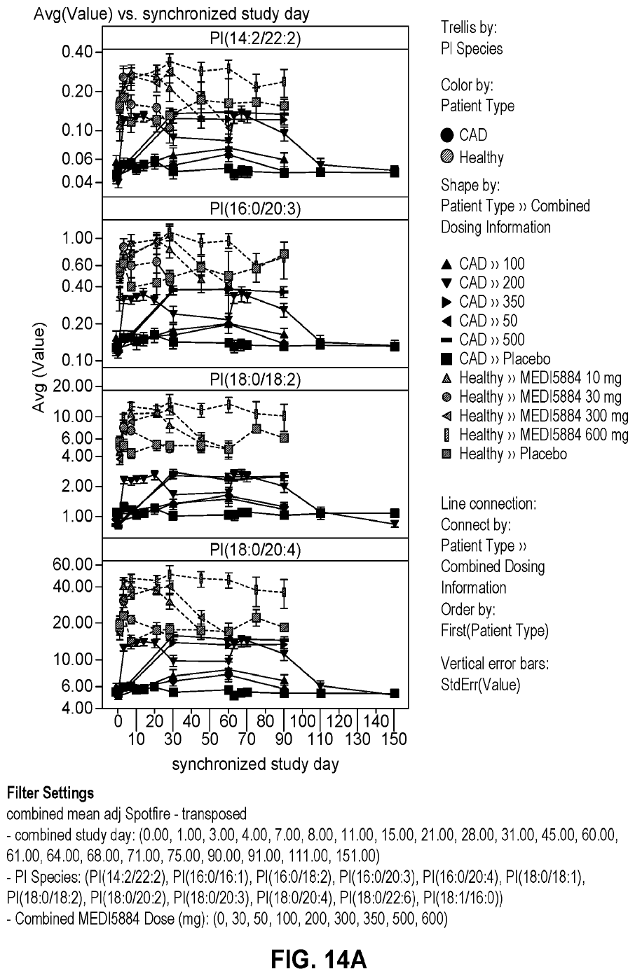

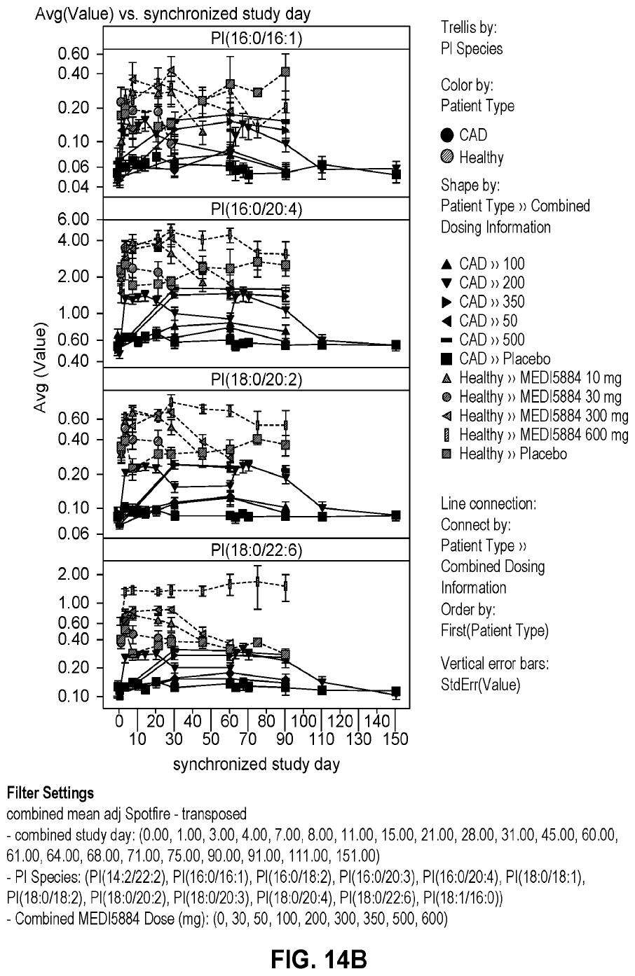

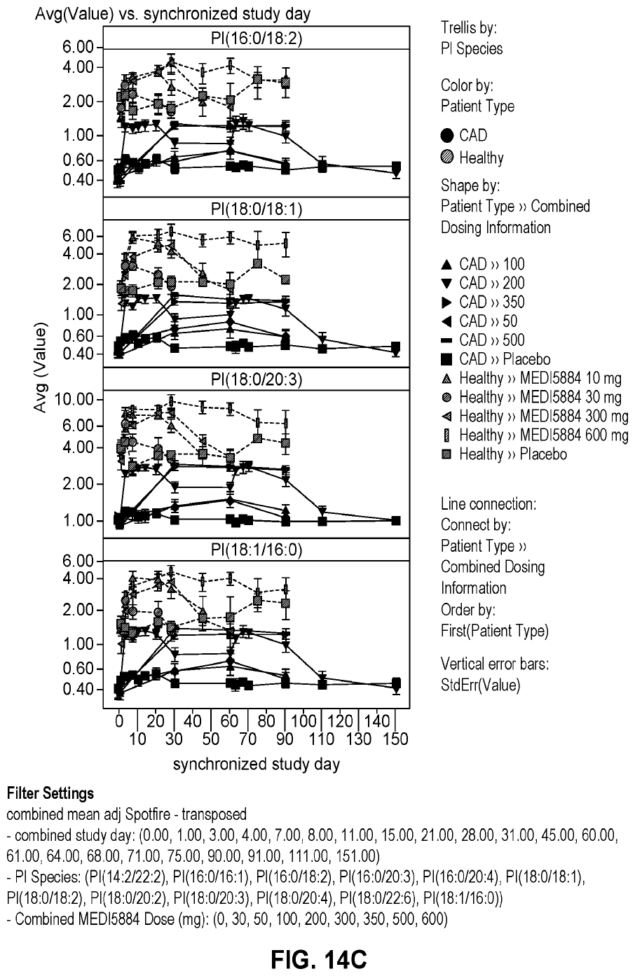

Clinical samples were also obtained and analyzed from healthy volunteers who received MEDI5884. Some variability between batches was observed. In order to correct for the batch-to-batch variability and explore the possibility of bridging the data between the clinical studies with CAD patients and the clinical studies with healthy volunteers, the SERRF normalization algorithm (Fan S., et al. Anal Chem March 5; 91 5:3590-6 (2019)) was evaluated, and found to outperform other normalization methods evaluated for this dataset. The results, shown in FIGS. 13A-C, 14A-C, 15, and 16, demonstrate that PI levels were markedly higher in healthy subjects compared to MEDI5884-untreated CAD patients across PI species. The difference was determined to be statistically significant by 2-tailed heteroscedastic t-test (p<0.0005 for 12 PI species). In the placebo arm of both clinical studies, PI levels remained relatively stable over a period of several months, and the lower levels in CAD patients were maintained relative to healthy volunteers in the 12 PI species examined.

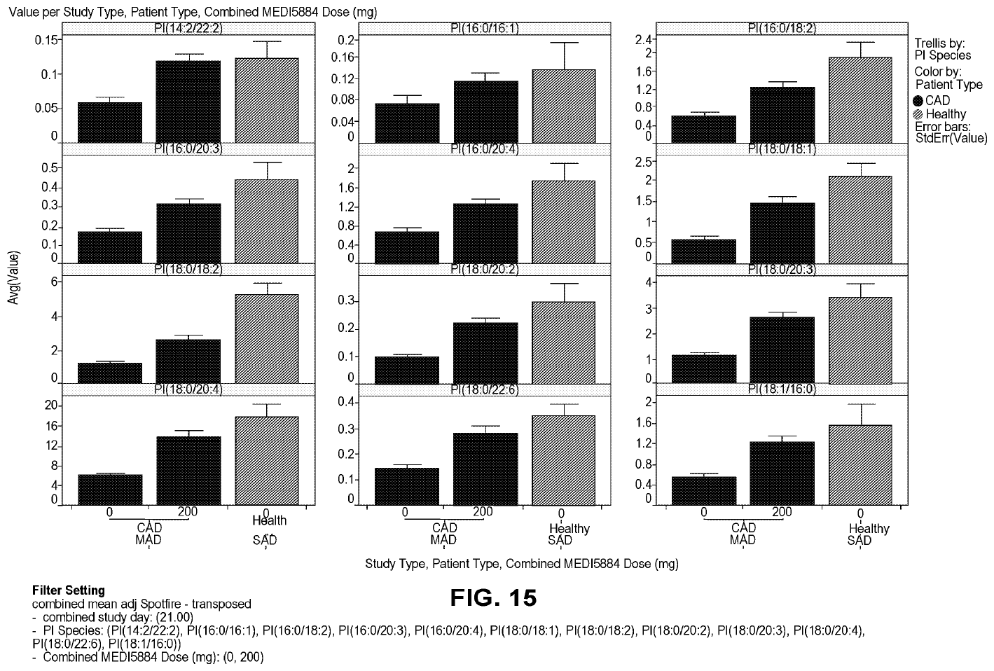

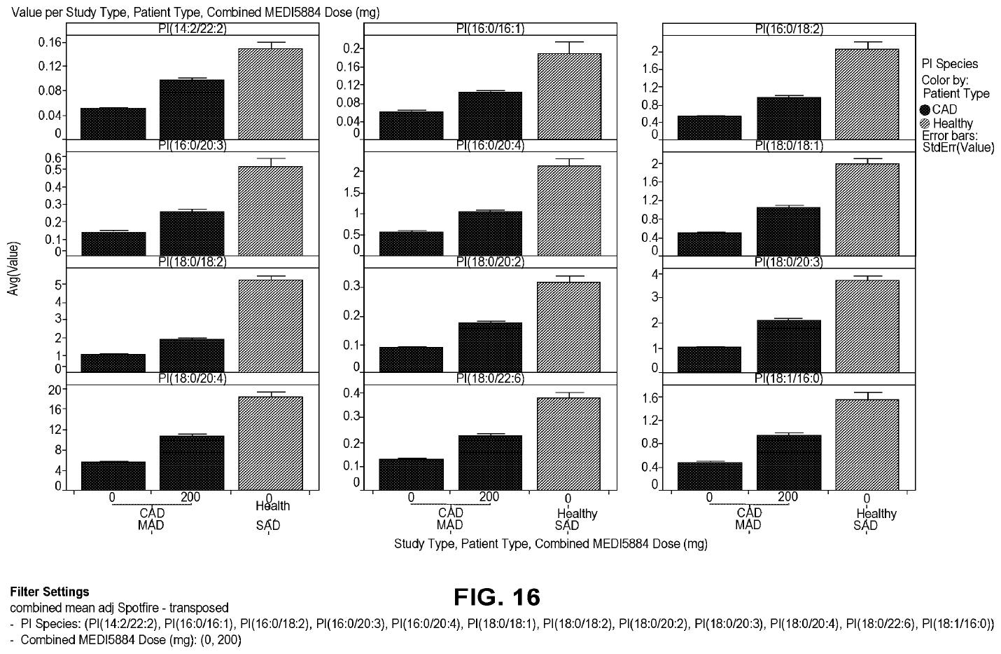

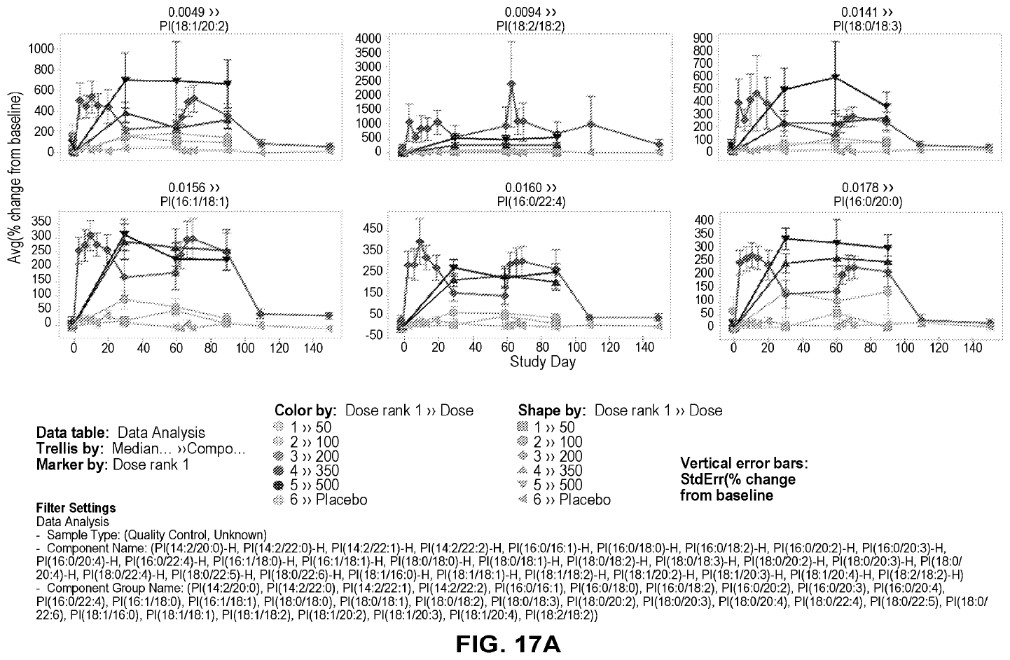

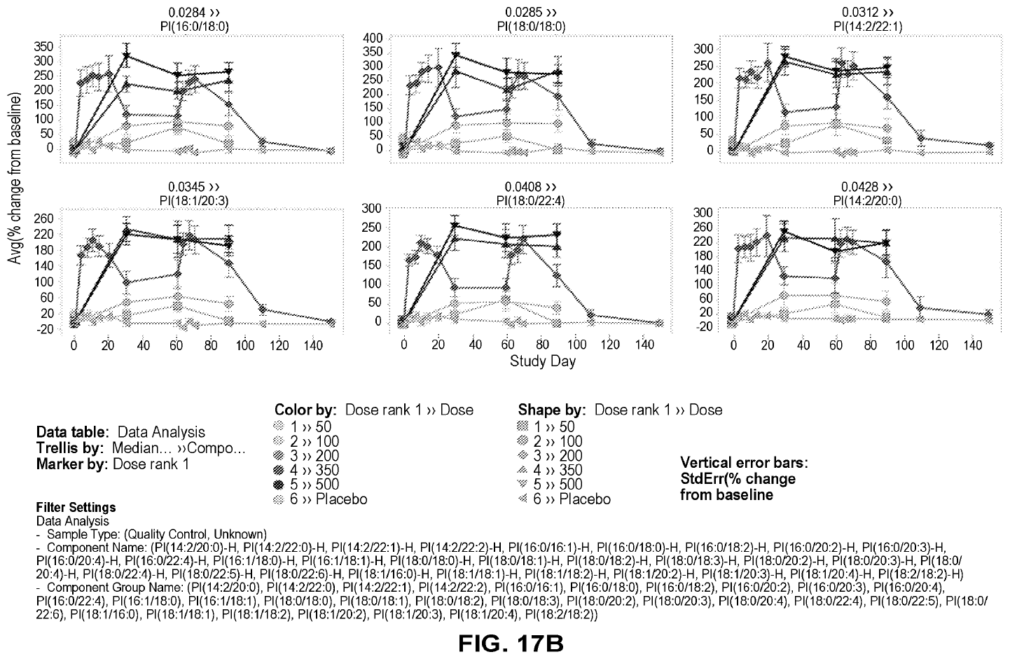

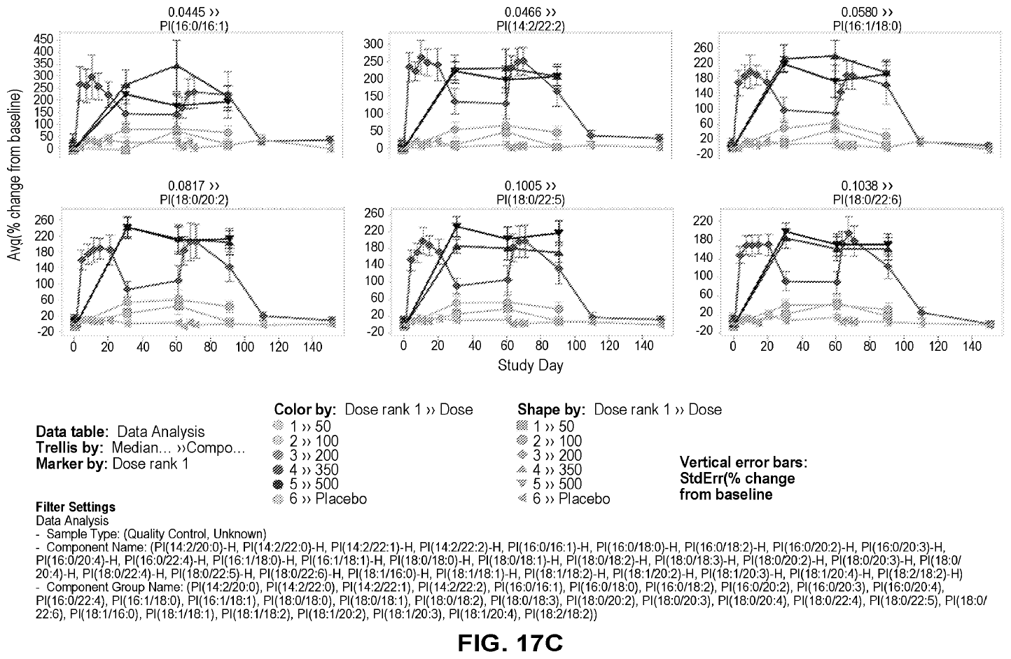

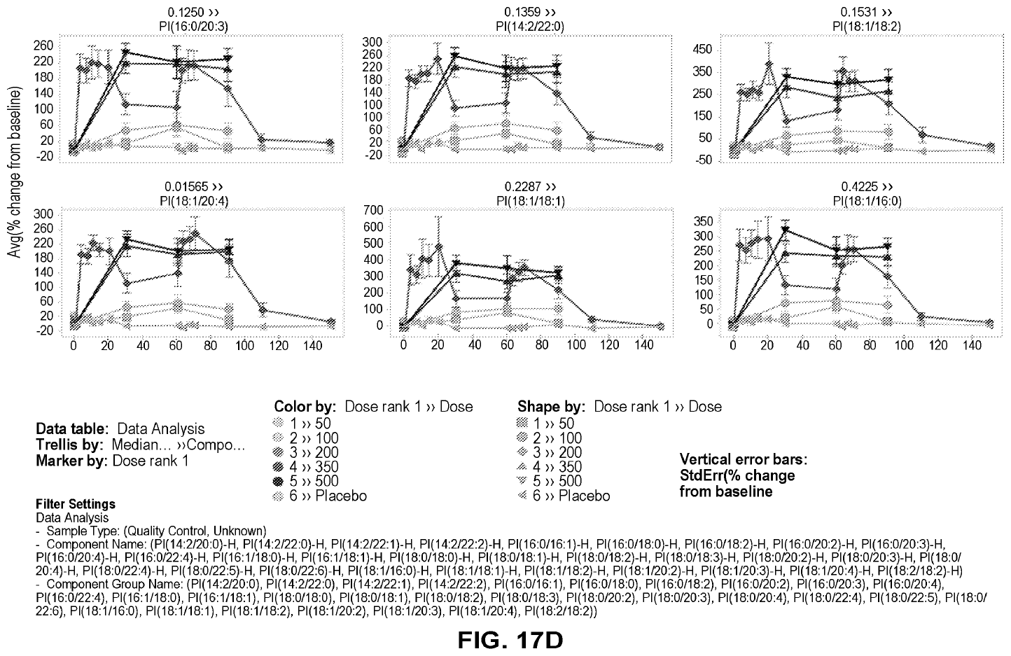

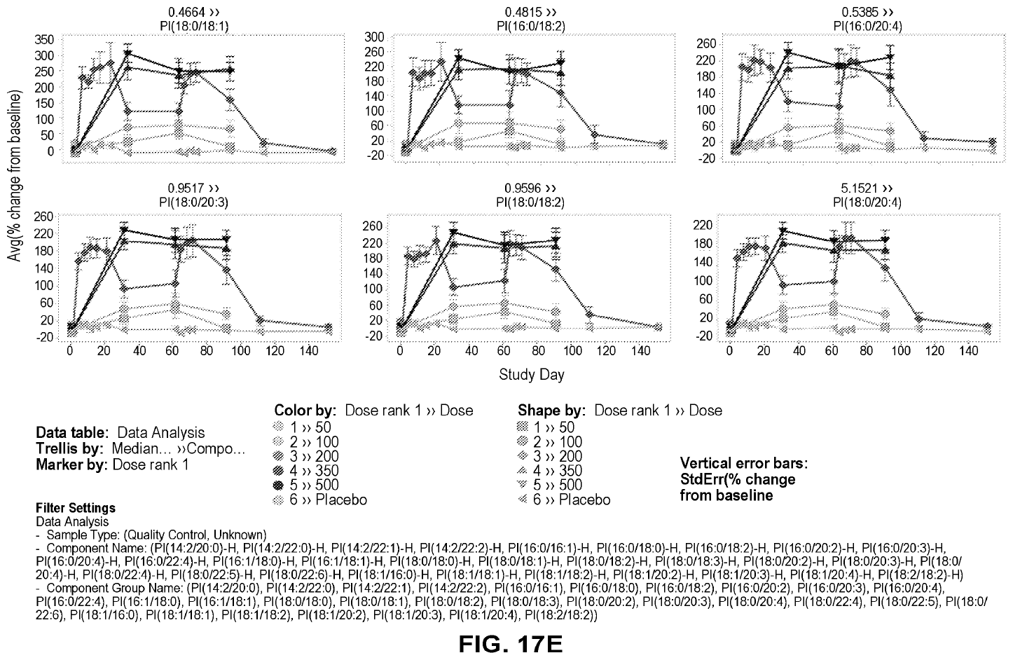

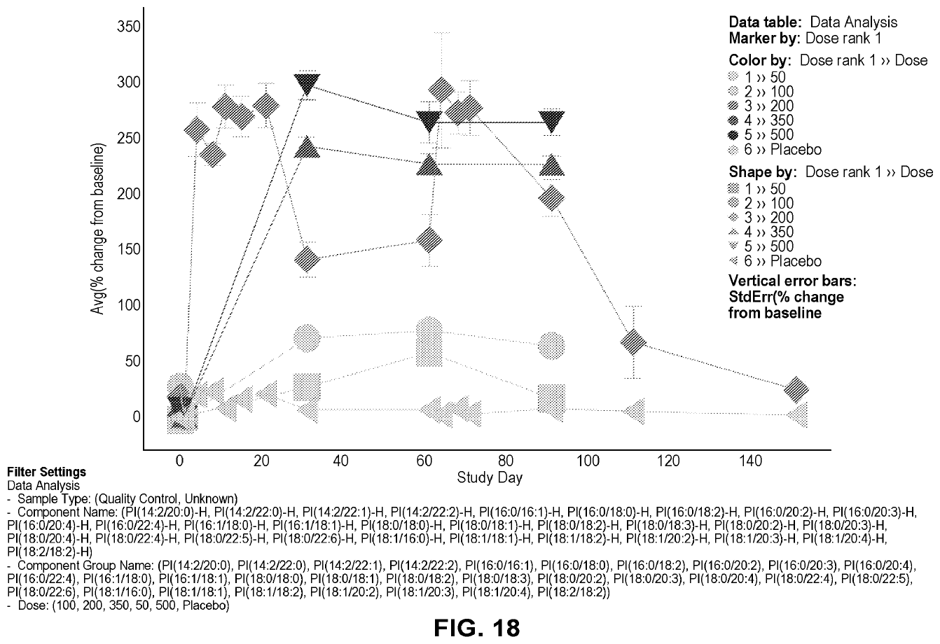

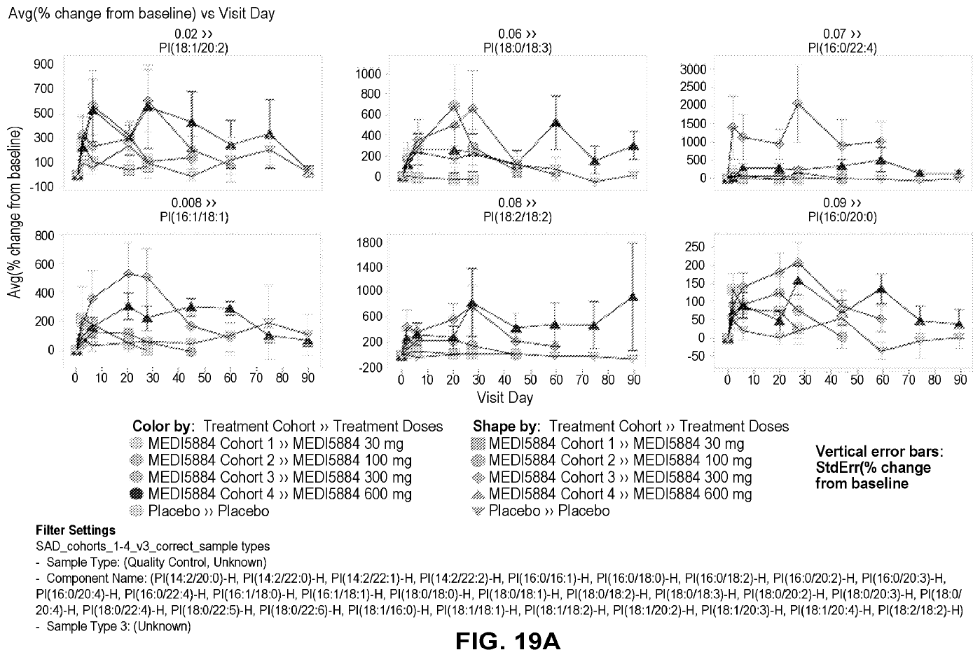

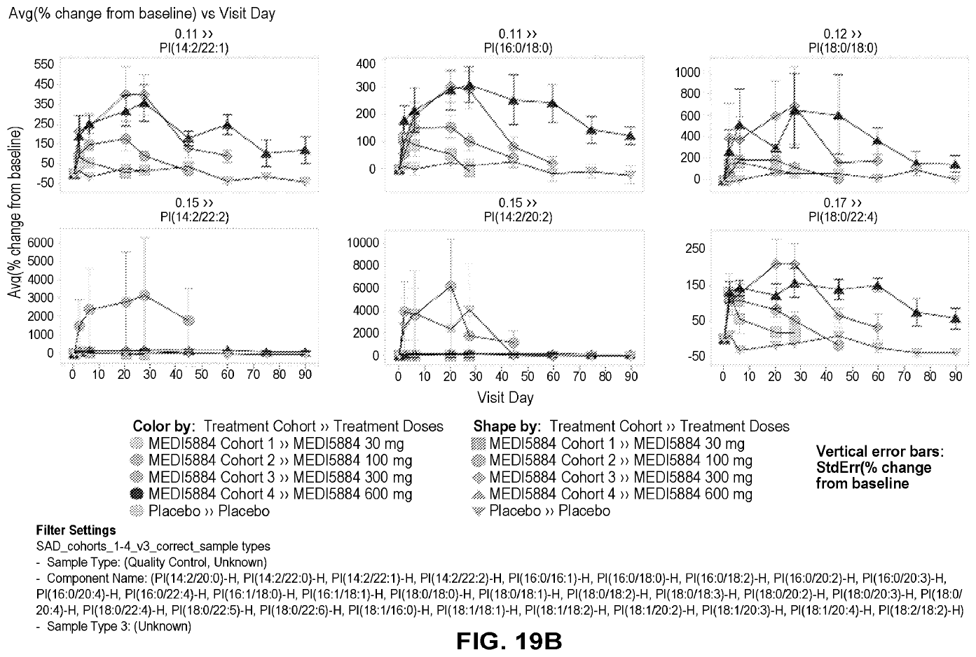

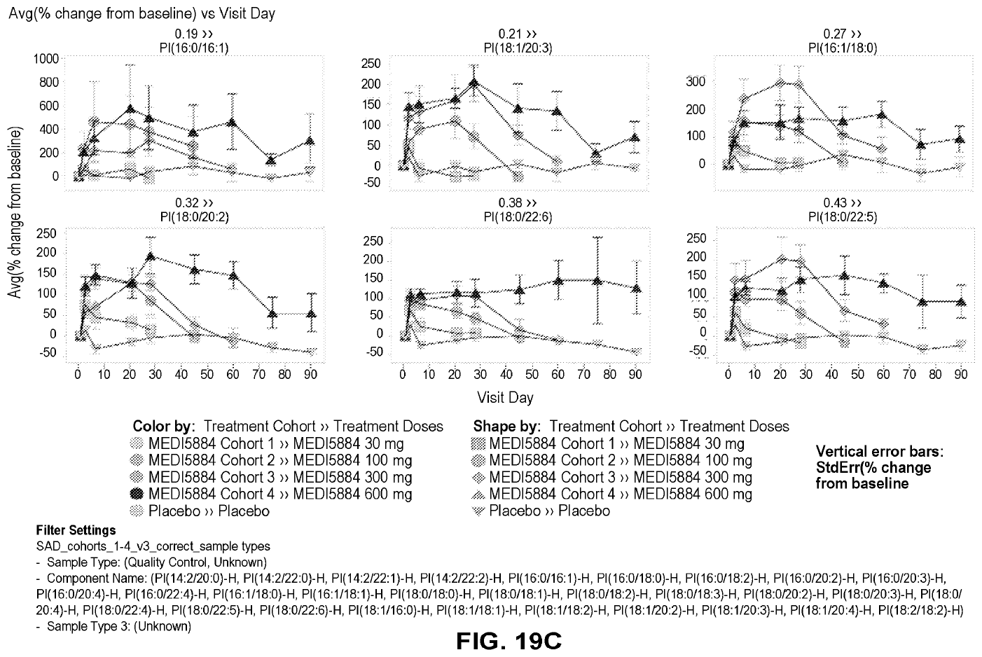

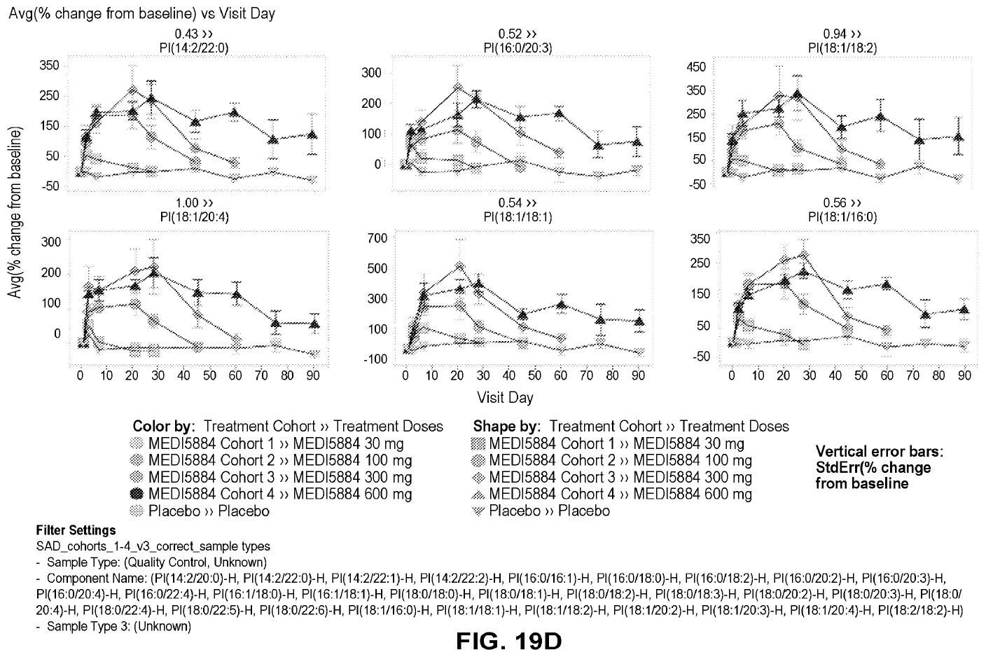

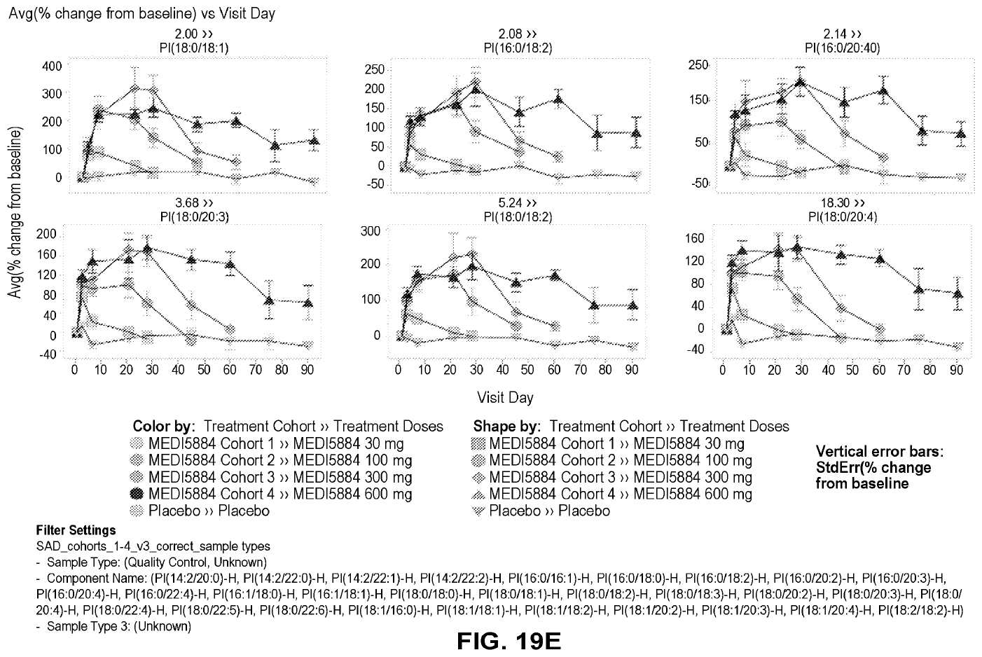

Levels of various PI species vs. visit day over time in healthy patient and patients with CAD are presented in FIGS. 13A-C and 14A-C. A comparison of the median PI species levels in healthy volunteers and CAD patients on Day 21 is shown in FIG. 15, and a comparison of the median PI species levels across all days is shown in FIG. 16. As demonstrated in FIGS. 13A-C, 14A-C, 15, and 16, after 3 monthly subcutaneous (SC) doses of MEDI5884, most plasma PI species dose-dependently increased relative to placebo. The duration of increases in plasma PI appeared to correlate with MEDI5884 exposure. For most PI species, increases in PI levels reached saturation at the MEDI5884 350 mg dose level, and the MEDI5884 500 mg dose level did not result in further PI increases relative to baseline. However, on Day 91 at the MEDI5884 200 mg dose level, PI increases approached saturation levels observed in higher dose cohorts. The percent change from baseline for each PI species varied from ˜1000% for less abundant species to ˜100-200% for more abundant species for both CAD and healthy volunteer subjects. (See FIGS. 17A-E, 18, and 19A-E; Table 18, summarizing the data obtained across all PI species at the indicated doses and days of visit; and Tables 19A-19D, showing the data obtained for individual PI species.) The average change across PI species for CAD patients reached maximum increases of ˜250-300% for doses ≥200 mg.

TABLE 18
PI Levels Across All PI Species

| | | | Avg (% | | |
|---|---|---|---|---|---|
| | | Avg (Peak | change | StdErr (Peak | StdErr (% |
| | Study | Area Ratio | from | Area Ratio | change from |
| Dose | Day | (PAR)) | baseline) | (PAR)) | baseline) |
| 100 | 0 | 0.3991 | 27 | 0.0484 | 4.5 |
| 100 | 1 | 0.3334 | 0 | 0.0366 | 0 |
| 100 | 31 | 0.4976 | 70 | 0.0622 | 5.7 |
| 100 | 61 | 0.5556 | 77 | 0.0783 | 6 |
| 100 | 91 | 0.4548 | 63 | 0.0544 | 5.9 |
| 200 | 0 | 0.3429 | 18 | 0.0426 | 4.7 |
| 200 | 1 | 0.3395 | 0 | 0.042 | 0 |
| 200 | 4 | 0.8886 | 257 | 0.1035 | 24.4 |
| 200 | 8 | 0.9098 | 235 | 0.1174 | 9.9 |
| 200 | 11 | 0.9625 | 278 | 0.1126 | 19.6 |
| 200 | 15 | 0.9704 | 269 | 0.1145 | 18.3 |
| 200 | 21 | 0.9618 | 279 | 0.1199 | 19.9 |
| 200 | 31 | 0.6674 | 141 | 0.0857 | 15.7 |
| 200 | 61 | 0.6721 | 158 | 0.0891 | 23.4 |
| 200 | 64 | 0.9422 | 293 | 0.1154 | 51.8 |
| 200 | 68 | 1.0074 | TIT | 0.1281 | 18.8 |
| 200 | 71 | 0.9873 | 277 | 0.1213 | 24.5 |
| 200 | 91 | 0.7736 | 196 | 0.1013 | 16.5 |
| 200 | 111 | 0.4181 | 67 | 0.0556 | 32.2 |
| 200 | 151 | 0.3469 | 24 | 0.0434 | 6.9 |
| 350 | 0 | 0.3666 | 10 | 0.0455 | 2.2 |
| 350 | 1 | 0.3366 | 0 | 0.0371 | 0 |
| 350 | 31 | 0.9632 | 242 | 0.1025 | 7.9 |
| 350 | 61 | 0.9403 | 227 | 0.1106 | 9.1 |
| 350 | 91 | 0.9333 | 226 | 0.1038 | 8.1 |
| 50 | 0 | 0.3549 | −3 | 0.0448 | 2 |
| 50 | 1 | 0.3923 | 0 | 0.043 | 0 |
| 50 | 31 | 0.4681 | 26 | 0.0539 | 3.8 |
| 50 | 61 | 0.5413 | 57 | 0.0668 | 4.4 |
| 50 | 91 | 0.4066 | 16 | 0.0442 | 3 |
| 500 | 0 | 0.3461 | 8 | 0.0425 | 3.3 |
| 500 | 1 | 0.3335 | 0 | 0.0367 | 0 |
| 500 | 31 | 1.0735 | 297 | 0.1155 | 13.1 |
| 500 | 61 | 1.0017 | 264 | 0.1229 | 18.5 |
| 500 | 91 | 0.9895 | 264 | 0.1073 | 12.2 |
| Placebo | 0 | 0.3739 | −1 | 0.0402 | 2 |
| Placebo | 1 | 0.3831 | 0 | 0.0393 | 0 |
| Placebo | 4 | 0.4247 | 19 | 0.0447 | 2.4 |
| Placebo | 8 | 0.4246 | 23 | 0.0454 | 2.9 |
| Placebo | 11 | 0.3911 | 7 | 0.0438 | 2.2 |
| Placebo | 15 | 0.4043 | 16 | 0.0434 | 2.4 |
| Placebo | 21 | 0.4268 | 20 | 0.0445 | 2.4 |
| Placebo | 31 | 0.3701 | 6 | 0.039 | 2.2 |
| Placebo | 61 | 0.3815 | 6 | 0.0405 | 2 |
| Placebo | 64 | 0.3597 | 2 | 0.0384 | 2.6 |
| Placebo | 68 | 0.3787 | 7 | 0.0403 | 2.1 |
| Placebo | 71 | 0.3746 | 3 | 0.0402 | 1.8 |
| Placebo | 91 | 0.3664 | 7 | 0.0396 | 2.1 |
| Placebo | 111 | 0.3654 | 5 | 0.0388 | 2.1 |
| Placebo | 151 | 0.3683 | 1 | 0.0387 | 1.9 |

TABLE 19A
Change in Levels of Individual PI Species

| | | PI | PI | PI | PI | PI | PI | PI | PI | PI |
|---|---|---|---|---|---|---|---|---|---|---|
| | | (14:2/20:0) | (14:2/22:0) | (14:2/22:1) | (14:2/22:2) | (16:0/16:1) | (16:0/18:0) | (16:0/18:2) | (16:0/20:2) | (16:0/20:3) |
| | | Avg (% | Avg (% | Avg (% | Avg (% | Avg (% | Avg (% | Avg (% | Avg (% | Avg (% |
| | | change | change | change | change | change | change | change | change | change |
| | Study | from | from | from | from | from | from | from | from | from |
| Dose | Day | baseline) | baseline) | baseline) | baseline) | baseline) | baseline) | baseline) | baseline) | baseline) |
| 100 | 31 | 72 | 58 | 77 | 54 | 79 | 82 | 68 | 136 | 49 |
| 100 | 61 | 73 | 72 | 84 | 65 | 83 | 98 | 69 | 105 | 65 |
| 100 | 91 | 57 | 50 | 70 | 45 | 65 | 81 | 52 | 136 | 48 |
| 200 | 31 | 128 | 114 | 116 | 136 | 145 | 120 | 117 | 129 | 116 |
| 200 | 61 | 121 | 128 | 132 | 130 | 142 | 116 | 117 | 139 | 108 |
| 200 | 91 | 167 | 156 | 161 | 164 | 221 | 156 | 149 | 215 | 157 |
| 350 | 31 | 235 | 229 | 264 | 229 | 265 | 225 | 211 | 248 | 221 |
| 350 | 61 | 233 | 209 | 227 | 232 | 345 | 200 | 213 | 265 | 218 |
| 350 | 91 | 219 | 216 | 235 | 208 | 217 | 236 | 205 | 252 | 205 |
| 50 | 31 | 21 | 24 | 24 | 11 | −4 | 25 | 17 | 5 | 17 |
| 50 | 61 | 48 | 44 | 80 | 48 | 72 | 77 | 47 | 58 | 57 |
| 50 | 91 | 12 | 13 | 33 | 5 | 18 | 23 | 12 | 3 | 6 |
| 500 | 31 | 252 | 259 | 279 | 224 | 226 | 321 | 242 | 341 | 247 |
| 500 | 61 | 197 | 226 | 237 | 199 | 178 | 253 | 207 | 324 | 224 |
| 500 | 91 | 221 | 232 | 247 | 209 | 194 | 266 | 229 | 306 | 231 |
| Placebo | 31 | 7 | −1 | −3 | 5 | 23 | 1 | 6 | 15 | 9 |
| Placebo | 61 | 8 | −1 | 2 | 10 | 23 | −4 | 6 | 8 | 7 |
| Placebo | 91 | 6 | 2 | 7 | 3 | 11 | 2 | 2 | 17 | 3 |

TABLE 19B
Change in Levels of Individual PI Species

| | | PI | PI | PI | PI | PI | PI | PI | PI | PI |
|---|---|---|---|---|---|---|---|---|---|---|
| | | (16:0/20:4) | (16:0/22:4) | (16:1/18:0) | (16:1/18:1) | (18:0/18:0) | (18:0/18:1) | (18:0/18:2) | (18:0/18:3) | (18:0/20:2) |
| | | Avg (% | Avg (% | Avg (% | Avg (% | Avg (% | Avg (% | Avg (% | Avg (% | Avg (% |
| | | change | change | change | change | change | change | change | change | change |
| | Study | from | from | from | from | from | from | from | from | from |
| Dose | Day | baseline) | baseline) | baseline) | baseline) | baseline) | baseline) | baseline) | baseline) | baseline) |
| 100 | 31 | 56 | 68 | 52 | 89 | 92 | 70 | 56 | 64 | 55 |
| 100 | 61 | 61 | 67 | 65 | 66 | 100 | 79 | 65 | 69 | 62 |
| 100 | 91 | 47 | 45 | 30 | 27 | 97 | 65 | 43 | 74 | 44 |
| 200 | 31 | 120 | 159 | 99 | 167 | 122 | 121 | 108 | 224 | 87 |
| 200 | 61 | 108 | 147 | 91 | 180 | 149 | 121 | 123 | 133 | 109 |
| 200 | 91 | 149 | 273 | 163 | 254 | 196 | 159 | 153 | 233 | 143 |
| 350 | 31 | 203 | 221 | 231 | 288 | 287 | 263 | 219 | 229 | 241 |
| 350 | 61 | 207 | 240 | 239 | 268 | 220 | 236 | 207 | 230 | 214 |
| 350 | 91 | 185 | 210 | 195 | 259 | 288 | 258 | 215 | 268 | 203 |
| 50 | 31 | 13 | 12 | 13 | 17 | 27 | 25 | 24 | 43 | 27 |
| 50 | 61 | 49 | 53 | 47 | 52 | 52 | 52 | 42 | 107 | 45 |
| 50 | 91 | 9 | 18 | 9 | 9 | 4 | 8 | 13 | 75 | 6 |
| 500 | 31 | 241 | 277 | 217 | 312 | 343 | 307 | 250 | 489 | 242 |
| 500 | 61 | 207 | 231 | 173 | 228 | 283 | 251 | 215 | 586 | 209 |
| 500 | 91 | 228 | 259 | 192 | 225 | 277 | 248 | 228 | 358 | 213 |
| Placebo | 31 | 6 | 17 | 10 | 10 | −1 | −10 | −2 | 9 | 2 |
| Placebo | 61 | 9 | 6 | 10 | −5 | −6 | −8 | 1 | 16 | 4 |
| Placebo | 91 | 3 | 2 | −1 | 9 | 12 | −2 | 0 | 5 | 0 |

TABLE 19C
Change in Levels of Individual PI Species

| | | PI | PI | PI | PI | PI | PI | PI | PI | PI |
|---|---|---|---|---|---|---|---|---|---|---|
| | | (18:0/20:3) | (18:0/20:4) | (18:0/22:4) | (18:0/22:5) | (18:0/22:6) | (18:1/16:0) | (18:1/18:1) | (18:1/18:2) | (18:1/20:2) |
| | | Avg (% | Avg (% | Avg (% | Avg (% | Avg (% | Avg (% | Avg (% | Avg (% | Avg (% |
| | | change | change | change | change | change | change | change | change | change |
| | Study | from | from | from | from | from | from | from | from | from |
| Dose | Day | baseline) | baseline) | baseline) | baseline) | baseline) | baseline) | baseline) | baseline) | baseline) |
| 100 | 31 | 48 | 45 | 52 | 52 | 41 | 74 | 82 | 65 | 163 |
| 100 | 61 | 60 | 54 | 59 | 53 | 40 | 80 | 108 | 85 | 185 |
| 100 | 91 | 37 | 34 | 41 | 37 | 31 | 67 | 106 | 82 | 146 |
| 200 | 31 | 94 | 97 | 94 | 92 | 92 | 134 | 166 | 131 | 221 |
| 200 | 61 | 107 | 104 | 95 | 106 | 90 | 119 | 168 | 181 | 259 |
| 200 | 91 | 137 | 135 | 125 | 133 | 125 | 164 | 222 | 210 | 357 |
| 350 | 31 | 204 | 188 | 222 | 186 | 184 | 245 | 320 | 287 | 387 |
| 350 | 61 | 195 | 174 | 207 | 181 | 162 | 234 | 272 | 236 | 240 |
| 350 | 91 | 187 | 175 | 202 | 171 | 162 | 232 | 308 | 266 | 315 |
| 50 | 31 | 26 | 24 | 25 | 26 | 22 | 21 | 40 | 23 | 156 |
| 50 | 61 | 46 | 39 | 62 | 38 | 46 | 62 | 82 | 43 | 109 |
| 50 | 91 | 4 | 5 | 2 | 9 | 18 | 10 | 19 | 9 | 99 |
| 500 | 31 | 228 | 216 | 255 | 232 | 199 | 322 | 384 | 331 | 700 |
| 500 | 61 | 206 | 192 | 224 | 204 | 171 | 253 | 351 | 300 | 695 |
| 500 | 91 | 206 | 196 | 230 | 217 | 170 | 266 | 326 | 319 | 663 |
| Placebo | 31 | 0 | 1 | 12 | 10 | 8 | 3 | −10 | −9 | 46 |
| Placebo | 61 | 2 | 3 | 6 | 11 | 14 | 2 | −11 | −2 | 47 |
| Placebo | 91 | −2 | 1 | 2 | 6 | 5 | 5 | 12 | 6 | 21 |

TABLE 19D
Change in Levels of Individual PI Species

| | | PI | PI | PI |
|---|---|---|---|---|
| | | (18:1/20:3) | (18:1/20:4) | (18:2/18:2) |
| | Study | Avg (% | Avg (% | Avg (% |
| | Day | change | change | change |
| Dose | Study | from | from | from |
| Dose | Day | baseline) | baseline) | baseline) |
| 100 | 31 | 50 | 47 | 110 |
| 100 | 61 | 64 | 58 | 120 |
| 100 | 91 | 45 | 40 | 154 |
| 200 | 31 | 98 | 113 | 564 |
| 200 | 61 | 121 | 140 | 961 |
| 200 | 91 | 148 | 175 | 683 |
| 350 | 31 | 234 | 217 | 286 |
| 350 | 61 | 209 | 193 | 289 |
| 350 | 91 | 209 | 201 | 284 |
| 50 | 31 | 16 | 21 | 51 |
| 50 | 61 | 41 | 45 | 73 |
| 50 | 91 | 2 | 11 | 22 |
| 500 | 31 | 221 | 234 | 526 |
| 500 | 61 | 208 | 202 | 482 |
| 500 | 91 | 191 | 207 | 563 |
| Placebo | 31 | −3 | −5 | 18 |
| Placebo | 61 | −4 | −2 | 24 |
| Placebo | 91 | −4 | −8 | 81 |

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes.

Other embodiments are within the following claims.

1.-98. (canceled)

99. A method of treating cardiovascular disease or reducing atherosclerosis in a subject, the method comprising administering to the subject an antibody or antigen-binding fragment comprising a VH CDR1 comprising the amino acid sequence of SEQ ID NO:1, a VH CDR2 comprising the amino acid sequence of SEQ ID NO:2, a VH CDR3 comprising the amino acid sequence of SEQ ID NO:3, a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:6, wherein the administration of the antibody or antigen-binding fragment thereof:
(a) increases high-density lipoprotein cholesterol (HDL-C) in the subject,
(b) increases high-density lipoprotein (HDL) particle number in the subject,
(c) increases HDL particle size in the subject,
(d) increases HDL phospholipids in the subject,
(e) increases apolipoprotein A1 (ApoA1) in the subject,
(f) increases cholesterol efflux capacity (CEC) in the subject, and/or
(g) increases plasma phosphatidylinositol (PI) levels in the subject.

100. The method of claim 99, wherein the administration reduces the risk of cardiovascular death, non-fatal myocardial infarction (MI), non-fatal stroke, and/or coronary revascularization in a subject with prior acute coronary syndrome (ACS).

101. The method of claim 100, wherein the administration preventing a secondary cardiovascular event in the subject.

102. The method of claim 101, wherein the administration reduces the risk of a major adverse cardiovascular event (MACE) in a subject.

103. The method of claim 99, wherein the antibody or antigen-binding fragment thereof is administered once a month.

104. The method of claim 99, wherein the antibody or antigen-binding fragment thereof is administered parenterally.

105. The method of claim 99, wherein the antibody or antigen-binding fragment thereof is administered subcutaneously.

106. The method of claim 99, wherein the antibody or antigen-binding fragment thereof is administered via an accessorized pre-filled syringe (APFS) or an auto-injector.

107. The method of claim 99, wherein the administration of the antibody or antigen-binding fragment thereof increases HDL-C in the subject by at least 30%.

108. The method of claim 99, wherein the administration of the antibody or antigen-binding fragment thereof increases ApoA1 in the subject by at least 30%.

109. The method of claim 99, wherein the administration of the antibody or antigen-binding fragment thereof increases HDL particle number in the subject by at least 5%.

110. The method of claim 99, wherein the administration of the antibody or antigen-binding fragment thereof increases HDL particle size in the subject by at least 3%.

111. The method of claim 99, wherein the administration of the antibody or antigen-binding fragment thereof increases HDL phospholipids in the subject by at least 50%.

112. The method of claim 99, wherein the administration of the antibody or antigen-binding fragment thereof increases cholesterol efflux capacity in the subject by at least 35%.

113. The method of claim 99, wherein the increased plasma PI levels are increased PI(14:2/20:0), PI(14:2/22:0), PI(14:2/22:1), PI(14:2/22:2), PI(16:0/16:1), PI(16:0/18:0), PI(16:0/18:2), PI(16:0/20:2), PI(16:0/20:3), PI(16: 0/20:4), PI(16:0/22:4), PI(16: 1/18:0), PI(16: 1/18:1), PI(18:0/18:0), PI(18:0/18:1), PI(18:0/18:2), PI(18:0/18:3), PI(18: 0/20:2), PI(18:0/20:3), PI(18:0/20:4), PI(18:0/22:4), PI(18:0/22:5), PI(18:0/22:6), PI(18: 1/16:0), PI(18: 1/18:1), PI(18: 1/18:2), PI(18: 1/20:2), PI(18: 1/20:3), PI(18: 1/20:4), and/or PI(18:2/18:2) levels.

114. The method of claim 99, wherein the subject has cardiovascular disease selected from the group consisting of coronary artery disease, coronary heart disease, chronic arterial disease, cerebrovascular disease, atherosclerotic cardiovascular disease and peripheral artery disease.

115. The method of claim 114, wherein the subject is receiving statin therapy.

116. The method of claim 114, wherein the subject has triglyceride levels <500 mg/dL prior to the administration of the antibody or antigen-binding fragment.

117. The method of claim 99, wherein the antibody or antigen-binding fragment thereof comprises a VH comprising the amino acid sequence set forth in SEQ ID NO:7 and/or a VL comprising the amino acid sequence set forth in SEQ ID NO:8.

118. The method claim 99, further comprising administering an inhibitor of PCSK9, wherein the administration of the antibody or antigen-binding fragment thereof and the administration of the inhibitor of PCSK9 are simultaneous or sequential.

119. The method one of claim 118, wherein the inhibitor of PCSK9 is an anti-PCSK9 antibody or antigen-binding fragment thereof, wherein the anti-PCSK9 antibody is selected from the group consisting of HS9, evolocumab, alirocumab and bococizumab.
